(19) United States
(12) Patent Application Publication
(10) Pub. No.: US 2020/0323851 A1
(43) Pub. Date: Oct. 15, 2020

(54) ALK5 INHIBITORS FOR TREATING MYELODYSPLASTIC SYNDROME

(71) Applicant: TOLERO PHARMACEUTICALS, INC., Lehi, UT (US)

(72) Inventors: David J. BEARSS, Alpine, UT (US); Steven L. WARNER, Sandy, UT (US)

(21) Appl. No.: 16/740,219

(22) Filed: Jan. 10, 2020

Related U.S. Application Data
(60) Provisional application No. 62/790,961, filed on Jan. 10, 2019.

(51) Int. Cl. A61K 31/506; A61K 9/00; A61K 9/48

(52) U.S. Cl. CPC A61K 31/506; A61K 9/4825; A61K 9/0053

(57) ABSTRACT

Described herein are methods for treating ALK5-mediated disease including myelodysplastic syndrome (MDS), anemia and anemia of chronic disease. Also provided are methods of inhibiting ALK5.

### CROSS-REFERENCE TO RELATED APPLICATION

This application claims benefit under 35 U.S.C. § 119(e) of U.S. Provisional Patent Application No. 62/790,961, filed Jan. 10, 2019, which is incorporated herein by reference in its entirety.

### FIELD OF THE INVENTION

The present disclosure relates to compounds, compositions comprising such compounds, and their use for the treatment of myelodysplastic syndrome and anemia of chronic disease.

### BACKGROUND

Myelodysplastic syndrome (MDS) is a collection of hematological conditions caused by abnormal blood-forming cells in bone marrow. These abnormal blood-forming cells form defective blood cells that die prematurely or are destroyed, leading to a shortage of blood cells. Most commonly, MDS results in a shortage of red blood cells, but other types of blood cells can be affected.

There are several types of MDS. For example, MDS may be triggered by an external cause (e.g., radiation and chemotherapy), which is referred to as “secondary MDS.” Secondary MDS is usually associated with multiple chromosomal abnormalities in cells in the bone marrow, and is more likely to progress to AML. If an external cause triggering the MDS is not identified, the MDS is referred to as “primary MDS.”

Anemia is the predominant cause of morbidity and quality of life impairment in subjects, in particular those with lower-risk (LR)-MDS (e.g., very low-risk, low-risk, or intermediate-risk MDS), and there are very limited therapy options for these subjects, especially after failure of erythropoiesis stimulating agents (ESAs). In approximately one third of cases, MDS progresses to acute myeloid leukemia (AML).

Anemia of chronic disease (ACD) is a form of anemia seen in chronic infection, chronic immune activation, and malignancy. These conditions all produce elevation of Interleukin-6, which stimulates hepcidin production and release from the liver, which in turn reduces the iron carrier protein ferroportin so that access of iron to the circulation is reduced. Other mechanisms may also play a role, such as reduced erythropoiesis. ACD is also referred to as anemia of chronic inflammation.

The transforming growth factor (TGF)-β superfamily comprises more than 30 soluble growth factors that play a central role in erythropoiesis and are part of a tightly regulated myelosuppressive negative feedback loop under physiologic conditions. TGF-β receptor activation and phosphorylation trigger a regulatory circuit of activating and inhibitory SMAD proteins and increased activation of the TGF-β signaling pathway either by a loss of negative feedback or constitutive activation has been associated with the myelosuppression and ineffective erythropoiesis in myelodysplastic syndromes (MDS). Also, reduction in SMAD7 is a novel molecular alteration in MDS that leads to ineffective hematopoiesis by activating of TGF-β signaling in hematopoietic cells. (Zhou, et al., Cancer Res. 2011 Feb. 1; 71(3):955-63.)

Inhibition of ALK5 in these subjects has the potential to provide a real difference in treating ALK5 mediated diseases, improving their quality of life and may positively impact how they respond to therapy, radiation, or surgery.

Thus, it is an object of the present disclosure to provide alternative compositions and methods for increasing red blood cell levels in subjects in need thereof.

### SUMMARY

There remains a need for new treatments and therapies for TGFβ type I receptor kinase (ALK5) mediated disorders or diseases (e.g., anemia, myelodysplastic syndrome (MDS) and anemia of chronic disease (ACD)). The present disclosure provides an ALK5 inhibitor compound of structure (I), pharmaceutically acceptable salts and crystalline forms thereof, pharmaceutical compositions thereof and therapeutic combinations thereof. The present disclosure further provides methods of treating ALK5-mediated disorders or diseases (e.g., MDS), comprising administering to a subject in need thereof an effective amount of an ALK5 inhibitor (e.g., a compound of structure (I)).

One aspect of the present disclosure provides a compound of structure (I):

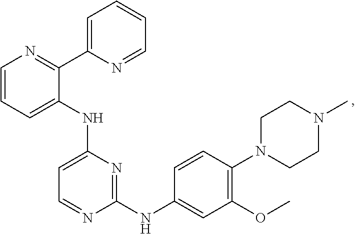 [SMILES: COc1cc(Nc2nccc(Nc3cccnc3-c3ccccn3)n2)ccc1N1CCN(C)CC1]

or a pharmaceutically acceptable salt, or prodrug thereof, for use in treating MDS, anemia, ACD or an ALK5-mediated disease in a subject with MDS or for reducing transfusion dependency or frequency in a subject, or for inhibiting ALK5.

Another aspect of the present disclosure provides a pharmaceutical composition comprising an effective amount of a compound of structure (I), or a pharmaceutically acceptable salt, or prodrug thereof, and one or more pharmaceutically acceptable carriers, for use in treating MDS.

In another aspect of present disclosure, a pharmaceutical combination is provided which comprises an effective amount of a compound of structure (I), or a pharmaceutically acceptable salt, or prodrug thereof, and one or more therapeutically active agents, for use in treating MDS, anemia, ACD or an ALK5-mediated disease.

In yet another aspect of present disclosure, a method is provided for treating MDS, anemia, ACD or an ALK5-mediated disease which comprises administering to a subject in need thereof an effective amount of a compound of structure (I), or a pharmaceutically acceptable salt, or prodrug thereof. Also provided are methods for determining the efficacy these methods.

### BRIEF DESCRIPTION OF THE DRAWINGS

In the figures, identical reference numbers identify similar elements. The sizes and relative positions of elements in the figures are not necessarily drawn to scale and some of these elements are arbitrarily enlarged and positioned to improve figure legibility. Further, the particular shapes of the elements as drawn are not intended to convey any information regarding the actual shape of the particular elements, and have been solely selected for ease of recognition in the figures.

FIG. 1 shows the effects of the compound of structure (I) on TGFβ induced SMAD 2/3 phosphorylation in Panc-1 cells.

FIG. 2 shows the effects of the compound of structure (I) on TGF β, BMP 6, BMP9 induced SMAD 2/3 phosphorylation in MOLM-13 cells.

FIG. 3 shows the effects of the compound of structure (I) on growth differentiation factor 11 (GDF 11) induced SMAD 2/3 phosphorylation in K562 cells.

FIG. 4A shows the vector used to transfect the RD cell line described in Example 2. FIG. 4B shows the results of the assay described in Example 2.

FIG. 5 shows the results of the assay described in Example 3.

FIGS. 6A-6C provide Table 2—a schedule of assessments for Phase I clinical trial.

FIGS. 7A-7C provide Table 3—a schedule of assessments for Phase II clinical trial.

FIG. 8 is an XRPD pattern of the compound of structure (I) mono-HCl salt Form A (812608-08-A1)

FIG. 9 provides XRPD overlays of the compound of structure (I) HCl salt crystal forms.

FIG. 10 is an XRPD overlay of the compound of structure (I) HCl salt Form A batches to demonstrate equivalence.

FIG. 11 shows TGA/DSC curves of the compound of structure (I) HCl Form A (812608-12-A).

FIG. 12 shows a DSC of the compound of structure (I) HCl Form A after heating (812608-12A_218C).

### DETAILED DESCRIPTION

Various (enumerated) embodiments of the disclosure are described herein. It will be recognized that features specified in each embodiment may be combined with other specified features to provide further embodiments of the present disclosure.

### Embodiment 1

A method for treating myelodysplastic syndrome (MDS) in a subject in need thereof, the method comprising:

administering an effective amount of a compound of structure (I):

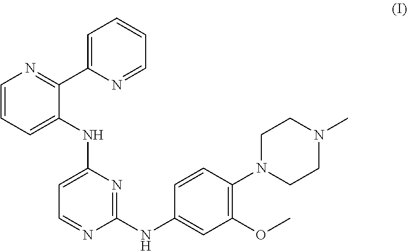 [SMILES: COc1cc(Nc2nccc(Nc3cccnc3-c3ccccn3)n2)ccc1N1CCN(C)CC1]

or a pharmaceutically acceptable salt, or prodrug thereof, to the subject.

### Embodiment 2

A method for treating anemia in a subject in need thereof, the method comprising:

administering an effective amount of a compound of structure (I):

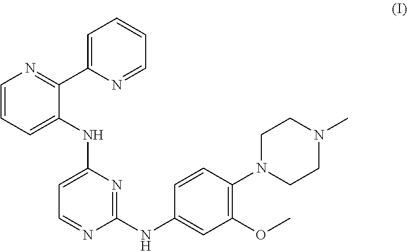 [SMILES: COc1cc(Nc2nccc(Nc3cccnc3-c3ccccn3)n2)ccc1N1CCN(C)CC1]

or a pharmaceutically acceptable salt, or prodrug thereof, to the subject.

### Embodiment 3

A method for treating anemia in a subject in need thereof, the method comprising:

administering an effective amount of a compound of structure (I):

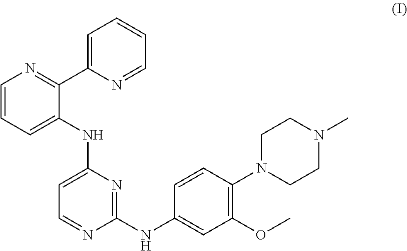 [SMILES: COc1cc(Nc2nccc(Nc3cccnc3-c3ccccn3)n2)ccc1N1CCN(C)CC1]

or a pharmaceutically acceptable salt, or prodrug thereof, to the subject; wherein the subject has very low, low or intermediate myelodysplastic syndrome (MDS).

### Embodiment 4

A method for treating anemia of chronic disease (ACD) in a subject in need thereof, the method comprising:

administering an effective amount of a compound of structure (I):

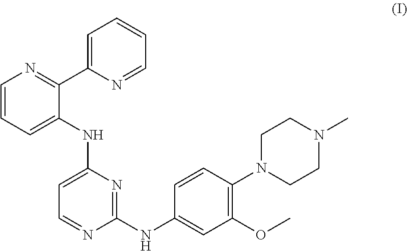 [SMILES: COc1cc(Nc2nccc(Nc3cccnc3-c3ccccn3)n2)ccc1N1CCN(C)CC1]

or a pharmaceutically acceptable salt, or prodrug thereof, to the subject.

### Embodiment 5

A method for reducing transfusion frequency in a subject in need thereof, the method comprising:

administering an effective amount of a compound of structure (I):

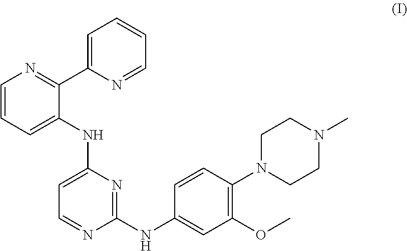 [SMILES: COc1cc(Nc2nccc(Nc3cccnc3-c3ccccn3)n2)ccc1N1CCN(C)CC1]

or a pharmaceutically acceptable salt, or prodrug thereof, to the subject.

### Embodiment 6

A method for reducing transfusion dependence in a subject in need thereof, the method comprising:

administering an effective amount of a compound of structure (I):

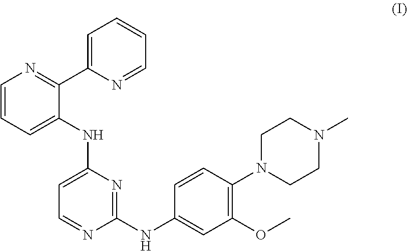 [SMILES: COc1cc(Nc2nccc(Nc3cccnc3-c3ccccn3)n2)ccc1N1CCN(C)CC1]

or a pharmaceutically acceptable salt, or prodrug thereof, to the subject.

### Embodiment 7

A method of treating an ALK5-mediated disorder, said method comprising administering an effective amount of a compound of structure (I):

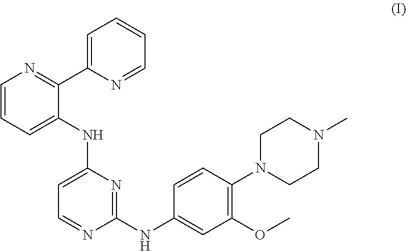 [SMILES: COc1cc(Nc2nccc(Nc3cccnc3-c3ccccn3)n2)ccc1N1CCN(C)CC1]

or a pharmaceutically acceptable salt, or prodrug thereof, to the subject; wherein the ALK5-mediated disorder is selected from anemia, myelodysplastic syndrome (MDS) and anemia of chronic disease (ACD).

### Embodiment 8

The method of any one of embodiments 1-7, wherein the method comprises improving one or more hematologic parameters in a subject, said improvement selected from decreasing myoblasts, increasing hemoglobin, increasing platelets, increasing neutrophils, decreasing hepcidin, reducing units of red blood cell transfused, reducing frequency of transfusion, and reducing transfusion dependence.

### Embodiment 9

The method of any one of embodiments 2, or 3-8, wherein the subject has myelodysplastic syndrome (MDS).

### Embodiment 10

The method of any one of embodiments 1-9, wherein the subject has anemia associated with myelodysplastic syndrome (MDS).

### Embodiment 11

The method of any one of embodiments 1-10, wherein the subject has transfusion dependent anemia associated with myelodysplastic syndrome (MDS).

### Embodiment 12

The method of any one of embodiments 1-11, wherein the subject has myelodysplastic syndrome (MDS) with single lineage dysplasia refractory anemia.

### Embodiment 13

The method of any one of embodiments 1-12, wherein the subject has myelodysplastic syndrome (MDS) with ring sideroblasts and is intolerant, resistant or refractory to luspatercept.

### Embodiment 14

The method of any one of embodiments 8-13, wherein increasing hemoglobin is defined as increasing hemoglobin i) to 10 g/dL or more; or ii) by 1.5 g/dL or more compared to an amount measured prior to administration of the compound of structure (I).

### Embodiment 15

The method of embodiment 14, wherein the increase in hemoglobin is maintained for 8 weeks or 12 weeks in the absence of red blood cell transfusions.

### Embodiment 16

The method of any one of embodiments 1-15, wherein the subject is transfusion dependent and wherein units of red blood cells transfused is reduced by 4 or more units compared to the units of red blood cells transfused for the same period of time prior to administration of the compound of structure (I).

### Embodiment 17

The method of embodiment 16, wherein the period of time is 8 weeks or 12 weeks.

### Embodiment 18

The method of any one of embodiments 8-17, wherein increasing platelets is defined as increasing the platelet count i) by 30×10⁹/L or more; or ii) to 75×10⁹/L or more.

### Embodiment 19

The method of embodiment 18, wherein the increase in platelets is maintained for 8 weeks or 12 weeks in the absence of red blood cell transfusions.

### Embodiment 20

The method of any one of embodiments 8-19, wherein increasing neutrophils is defined as increasing the neutrophil count i) by 0.5×10⁹/L or more or ii) to 1.0×10⁹/L or more.

### Embodiment 21

The method of embodiment 20, wherein the increase in neutrophil count is maintained for 8 weeks or 12 weeks in the absence of red blood cell transfusions.

### Embodiment 22

The method of any one of embodiments 8-21, wherein decreasing myoblasts is defined as decreasing myoblasts i) to be 5% or fewer of bone marrow cells; or ii) by 50% or more compared to a baseline amount measured prior to administration of the compound of structure (I).

### Embodiment 23

The method of embodiment 22, wherein the decrease in myoblasts is maintained for 8 weeks or 12 weeks.

### Embodiment 24

The method of any one of embodiments 8-23, wherein decreasing hepcidin is defined as decreasing hepcidin by 25% or more compared to a baseline amount measured prior to administration of the compound of structure (I).

### Embodiment 25

The method of any one of embodiments 1-24, wherein the method comprises preventing iron overload of the subject.

### Embodiment 26

The method of any one of embodiments 1-25, wherein the compound of structure (I) is formulated with one or more pharmaceutically acceptable carriers in a pharmaceutical composition.

### Embodiment 27

The method of any one of embodiments 1-26, wherein the pharmaceutically acceptable salt of the compound of structure (I) is pharmaceutically acceptable acid addition salt.

### Embodiment 28

The method of embodiment 27, wherein the pharmaceutically acceptable acid addition salt is a hydrochloric acid salt.

### Embodiment 29

The method of any one of embodiments 1-28, further comprising administering an effective amount of one or more therapeutically active agents.

### Embodiment 30

The method of embodiment 29, where the one or more therapeutically active agents comprise one or more anti-cancer agents, anti-allergic agents, anti-emetics, pain relievers, immunomodulators, cytoprotective agents, or a combination thereof.

### Embodiment 31

The method of embodiment 29 or 30, wherein the one or more therapeutically active agents is selected from the group consisting of: thalidomide, lenalidomide, azacitidine, and decitabine.

### Embodiment 32

The method of embodiment 29 or 30, wherein the one or more therapeutically active agents comprise a cyclin dependent kinase (CDK) inhibitor.

### Embodiment 33

The method of embodiment 32, wherein the CDK inhibitor is a CDK9 inhibitor.

### Embodiment 34

The method of embodiment 33, wherein the CDK9 inhibitor is alvocidib, or a prodrug thereof, dinaciclib, or a combination thereof.

### Embodiment 35

The method of embodiment 33 or 34, wherein the CDK9 inhibitor is alvocidib, or a prodrug thereof.

### Embodiment 36

The method of embodiment 34 or 35, wherein the prodrug of alvocidib is a phosphate prodrug.

### Embodiment 37

The method of any one of embodiments 1, 3 or 7-36, wherein the MDS is primary MDS.

### Embodiment 38

The method of any one of embodiments 1, 3 or 7-36, wherein the MDS is secondary MDS.

### Embodiment 39

The method of any one of embodiments 1, 3 or 7-36, wherein the MDS is high-risk MDS.

### Embodiment 40

The method of any one of embodiments 1, 3 or 7-36, wherein the MDS is very low-risk MDS, low-risk MDS or intermediate-risk MDS.

### Embodiment 41

The method of embodiment 40, wherein the MDS is very low-risk MDS.

### Embodiment 42

The method of embodiment 40, wherein the MDS is low-risk MDS.

### Embodiment 43

The method of embodiment 40, wherein the MDS is intermediate-risk MDS.

### Embodiment 44

The method of any one of embodiments 1-43, wherein the compound of structure (I) is administered as a maintenance dosage regime.

### Embodiment 45

The method of embodiment 44, wherein the compound of structure (I) is administered at a daily maintenance dosage regime comprising a dosage that is less than a maximum tolerated dose or a maximum administered dose.

### Embodiment 46

The method of embodiment 44 or 45, wherein the dosage is from 10 to 350 mg.

### Embodiment 47

The method of embodiment 46, wherein the dosage is 20 mg, 40 mg, 60 mg, 90 mg, 120 mg, 160 mg, 210 mg or 270 mg.

### Embodiment 48

The method of embodiment 46, wherein the dosage is in the range from 90-120 mg.

### Embodiment 49

The method of any one of embodiment 44-48, further comprising the steps of:

(a) administering a loading dose of a compound of structure (I):

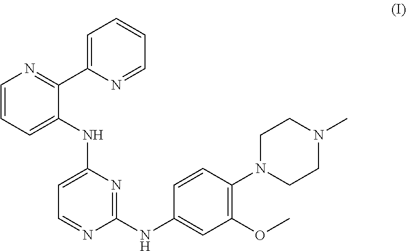 [SMILES: COc1cc(Nc2nccc(Nc3cccnc3-c3ccccn3)n2)ccc1N1CCN(C)CC1]

or a pharmaceutically acceptable salt, or prodrug thereof, to the subject; and

(b) determining if a hemoglobin level is above, at, or below a predetermined loading dose threshold or determining if a change in hemoglobin level is above, at, or below a predetermined amount, wherein:

(i) if the hemoglobin level is below the predetermined loading dose threshold or if the change in hemoglobin level is below the predetermined amount, then administering a subsequent loading dose and repeating steps a-b; or

(ii) if the hemoglobin level is at or above the predetermined loading dose threshold or if the change in hemoglobin level is at or above the predetermined amount, then administering the compound of structure (I) according to the maintenance dosage regime.

### Embodiment 50

The method of embodiment 49, wherein step b) further comprises a step of measuring a hemoglobin level.

### Embodiment 51

The method of embodiment 49 or 50, wherein the loading dose is from 20 mg to 350 mg.

### Embodiment 52

The method of any one of embodiments 49-51, wherein the predetermined loading dose threshold of hemoglobin is 0.5 g/dL or more.

### Embodiment 53

The method of any one of embodiments 49-51, wherein the predetermined amount of change of hemoglobin is 0.1 g/dL, 0.2 g/dL, 0.3 g/dL, 0.4 g/dL, 0.5 g/dL or more.

### Embodiment 54

The method of any one of embodiments 49-52, wherein the subsequent loading dose is increased by 20%, 30%, 50%, 75% or 100% compared to the loading dose administered in step a.

### Embodiment 55

The method of any one of embodiments 49-54, wherein the subsequent loading dose is increased by 10 mg.

### Embodiment 56

The method of any one of embodiments 44-55, further comprising the steps of:

(a) administering the maintenance dose;

(b) determining if a hemoglobin level is above, at, or below a predetermined maintenance dose threshold or determining if a change in hemoglobin level is above, at, or below a predetermined amount, wherein:

(i) if the hemoglobin level is below the predetermined maintenance dose threshold or if the change in hemoglobin level is below the predetermined amount, then administering a subsequent maintenance dose and repeating steps c-d; or

(ii) if the hemoglobin level is at or above the predetermined maintenance dose threshold or if the change in hemoglobin level is at or above the predetermined amount, then administering a reduced maintenance dose wherein the dosage is reduced by a predetermined amount, and optionally repeating steps c-d.

### Embodiment 57

The method of embodiment 56, wherein step d) further comprises a step of measuring a hemoglobin level from blood serum obtained from the subject.

### Embodiment 58

The method of embodiment 56, wherein the predetermined maintenance dose threshold of hemoglobin is 10 g/dL or more, wherein the increase is maintained over 12 weeks without red blood cell transfusions.

### Embodiment 59

The method of embodiment 56, wherein the predetermined amount of change of hemoglobin is 1.5 g/dL or more, wherein the change is determined from a baseline measurement.

### Embodiment 60

The method of embodiment 56 or 57, wherein the reduced maintenance dose is decreased by 2%, 5%, 10%, 20%, 30%, 50%, 75% or 100% compared to the maintenance dose administered in step d.

### Embodiment 61

The method of any one of embodiments 44-55, further comprising the steps of:

(a) administering the maintenance dose; and

(b) determining if a biomarker level is above, at or below a predetermined maintenance dose threshold or determining if a change in biomarker level is above, at, or below a predetermined amount, wherein:

(i) if the biomarker level is below the predetermined maintenance dose threshold or if the change in biomarker level is below the predetermined amount, then administering a subsequent maintenance dose and repeating steps c-d; or

(ii) if the biomarker level is at or above the predetermined maintenance dose threshold or if the change in biomarker level is at or above the predetermined amount, then administering a reduced maintenance dose wherein the dosage is reduced by a predetermined amount, and optionally repeating steps c-d.

### Embodiment 62

The method of embodiment 61, wherein step d) further comprises a step of measuring a biomarker level.

### Embodiment 63

The method of embodiment 61 or 62, wherein the biomarker is selected from hepcidin in serum and bone marrow aspirate; iron metabolism markers in serum selected from iron, ferritin, transferrin, soluble transferrin receptor [STR], and total iron binding capacity [TIBC]; cytokines in serum or plasma selected from CRP, EPO, IL-6, and TGF-beta 1; and indicators of inhibition of signal transduction pathways in bone marrow aspirates selected from phosphorylation of SMAD-1, 2, 3, 5 and 8 in PBMCs.

### Embodiment 64

The method of embodiment 63, wherein the biomarker is selected from cytokines in serum or plasma selected from CRP, EPO, IL-6, and TGF-beta 1; and indicators of inhibition of signal transduction pathways in bone marrow aspirates selected from phosphorylation of SMAD-1, 2, 3, 5 and 8 in PBMCs.

### Embodiment 65

The method of any one of embodiments 44-55, further comprising the steps of:

(a) administering the maintenance dose; and

(b) determining if a biomarker level is above, at or below a predetermined maintenance dose threshold or determining if a change in biomarker level is above, at, or below a predetermined amount, wherein:

(i) if the biomarker level is above a predetermined maintenance dose threshold or if the change in biomarker level is above the predetermined amount, then administering a subsequent maintenance dose and repeating steps c-d; or

(ii) if the biomarker level is at or below the predetermined maintenance dose threshold or if the change in biomarker level is at or below the predetermined amount, then administering a reduced maintenance dose wherein the dosage is reduced by a predetermined amount, and optionally repeating steps c-d.

### Embodiment 66

The method of embodiment 65, wherein the step d) further comprises a step of measuring a biomarker level.

### Embodiment 67

The method of embodiment 65 or 66, wherein the biomarker is selected from hepcidin in serum and bone marrow aspirate; iron metabolism markers in serum selected from iron, ferritin, transferrin, soluble transferrin receptor [STR], and total iron binding capacity [TIBC]; cytokines in serum or plasma selected from CRP, EPO, IL-6, and TGF-beta 1; and indicators of inhibition of signal transduction pathways in bone marrow aspirates selected from phosphorylation of SMAD-1, 2, 3, 5 and 8 in PBMCs.

### Embodiment 68

The method of embodiment 67, wherein the biomarker is selected from cytokines in serum or plasma selected from CRP, EPO, IL-6, and TGF-beta 1; and indicators of inhibition of signal transduction pathways in bone marrow aspirates selected from phosphorylation of SMAD-1, 2, 3, 5 and 8 in PBMCs.

### Embodiment 69

A method of determining the efficacy of treatment of the method of any one of embodiments 1-60, said method comprising the steps of:

(a) determining a baseline amount of hemoglobin in said subject;

(b) determining a change in hemoglobin from baseline after said administration step;

wherein if the hemoglobin has increased from baseline by 1.5 g/dL, the method of administering the compound of structure (I) for treatment is determined to be efficacious.

### Embodiment 70

A method of determining the efficacy of treatment of the method of any one of embodiments 1-60, said method comprising the steps of:

(a) determining a baseline level of hemoglobin in said subject;

(b) determining a subsequent level of hemoglobin after said administration step;

wherein if the hemoglobin level is 10 g/dL or more, the method of administering the compound of structure (I) for treatment is determined to be efficacious.

### Embodiment 71

A method of determining the efficacy of treatment of the method of any one of embodiments 1-70, said method comprising the steps of:

(a) determining a baseline amount of a biomarker in said subject;

(b) determining a change in a biomarker level from baseline after said administration step;

wherein if the biomarker has decreased or increased from baseline by a predetermined amount, the method of administering the compound of structure (I) for treatment is determined to be efficacious.

### Embodiment 72

The method of embodiment 70, wherein the biomarker is selected from hepcidin in serum and bone marrow aspirate; iron metabolism markers in serum selected from iron, ferritin, transferrin, soluble transferrin receptor [STR], and total iron binding capacity [TIBC]; cytokines in serum or plasma selected from CRP, EPO, IL-6, and TGF-beta 1; and indicators of inhibition of signal transduction pathways in bone marrow aspirates selected from phosphorylation of SMAD-1, 2, 3, 5 and 8 in PBMCs.

### Embodiment 73

The method of embodiment 72, wherein the biomarker is selected from cytokines in serum or plasma selected from CRP, EPO, IL-6, and TGF-beta 1; and indicators of inhibition of signal transduction pathways in bone marrow aspirates selected from phosphorylation of SMAD-1, 2, 3, 5 and 8 in PBMCs.

### Embodiment 74

The method of embodiment 72, wherein the biomarker is hepcidin in serum.

### Embodiment 75

A method of inhibiting ALK5, the method comprising administering a compound of structure (I):

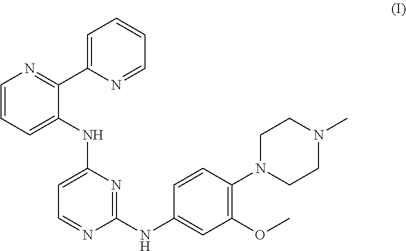 [SMILES: COc1cc(Nc2nccc(Nc3cccnc3-c3ccccn3)n2)ccc1N1CCN(C)CC1]

a pharmaceutically acceptable salt, or prodrug thereof.

### Embodiment 76

A method for inhibiting ALK5 activity in a subject, the method comprising administering an effective amount of a compound of structure (I):

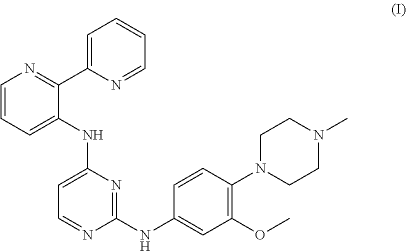 [SMILES: COc1cc(Nc2nccc(Nc3cccnc3-c3ccccn3)n2)ccc1N1CCN(C)CC1]

or a pharmaceutically acceptable salt, or prodrug thereof, to the subject.

### Embodiment 77

A method of inhibiting ALK5, comprising contacting cells expressing ALK5 with an effective amount of a compound of structure (I)

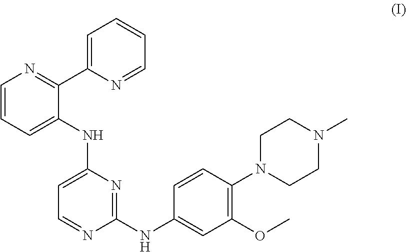 [SMILES: COc1cc(Nc2nccc(Nc3cccnc3-c3ccccn3)n2)ccc1N1CCN(C)CC1]

or a pharmaceutically acceptable salt, or prodrug thereof, to the subject.

### Embodiment 78

A method for inhibiting ALK5 activity in a cell, the method comprising administering to the cell a compound of structure (I)

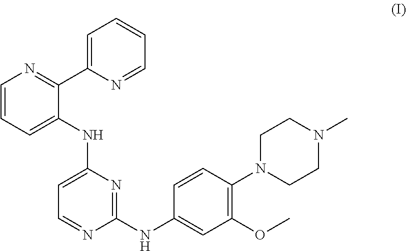 [SMILES: COc1cc(Nc2nccc(Nc3cccnc3-c3ccccn3)n2)ccc1N1CCN(C)CC1]

in an amount effective to inhibit ALK5.

### Embodiment 79

The method according to any one of embodiments 75-78, wherein inhibition is measured by pSMAD 2/3 phosphorylation.

### Embodiment 80

The method according to embodiment 79, wherein a measured IC50 is 280 nM or higher.

### Embodiment 81

The method according to any one of embodiments 75-78, wherein inhibition is measured by nanobret assay.

### Embodiment 82

The method according to embodiment 81, wherein a measured IC50 is 2.2 μM or more.

### Embodiment 83

The method according to any one of embodiments 75-78, wherein inhibition is measured by SMAD reporter (RDSR) assay.

### Embodiment 84

The method according to embodiment 83, wherein a measured IC50 is 250 nM or more.

### Embodiment 85

The method of any one of embodiments 1 to 84, wherein the compound of structure (I) is a crystalline salt.

### Embodiment 86

The method of embodiment 85, wherein the crystalline salt is an acid addition salt.

### Embodiment 87

The method of embodiment 86, wherein the acid addition salt is a hydrochloric acid salt.

### Embodiment 88

The method of embodiment 87, wherein the hydrochloric acid salt is monovalent.

### Embodiment 89

The method of any one of embodiments 85-88, wherein the crystalline salt form is anhydrous.

### Embodiment 90

The method of any one of embodiments 85-89, wherein the crystalline salt form comprises Form A.

### Embodiment 91

The method of any one of embodiments 85-90, wherein the crystalline salt form consists essentially of Form A.

### Embodiment 92

The method of any one of embodiments 85-91, wherein the crystalline salt form is essentially free from impurities.

### Embodiment 93

The method of any one of embodiments 85-92, wherein the crystalline salt form is in substantially pure form.

### Embodiment 94

The method of one of embodiments 85-93, wherein the crystalline salt form comprises Form A characterized by an x-ray diffraction pattern (XRPD) comprising one or more 20 values selected from: 13.53, 16.14, 17.67, 18.38, 24.96, and 28.18.

### Embodiment 95

The method of embodiment 94, wherein the form is characterized by two or more of the listed 20 values.

### Embodiment 96

The method of embodiment 94, wherein the form is characterized by three or more of the listed 20 values.

### Embodiment 97

The method of embodiment 94, wherein the form is characterized by four or more of the listed 20 values.

### Embodiment 98

The method of embodiment 94, wherein the form is characterized by five or more of the listed 20 values.

### Embodiment 99

The method of embodiment 94, wherein the form is characterized by all six of the listed 20 values.

### Embodiment 100

The method of embodiment 94, wherein an X-ray powder diffractometer is used in reflection mode with an X-ray wavelength of Cu kα, Kα1 (Å): 1.540598, Kα2 (Å): 1.544426, with a Kα2/Kα1 intensity ratio of 0.50, and an X-ray tube setting of 45 kV, 40 mA.

### Embodiment 101

The method of embodiment 94 or 100, wherein the 20 values are within +/−0.2 2θ.

### Embodiment 102

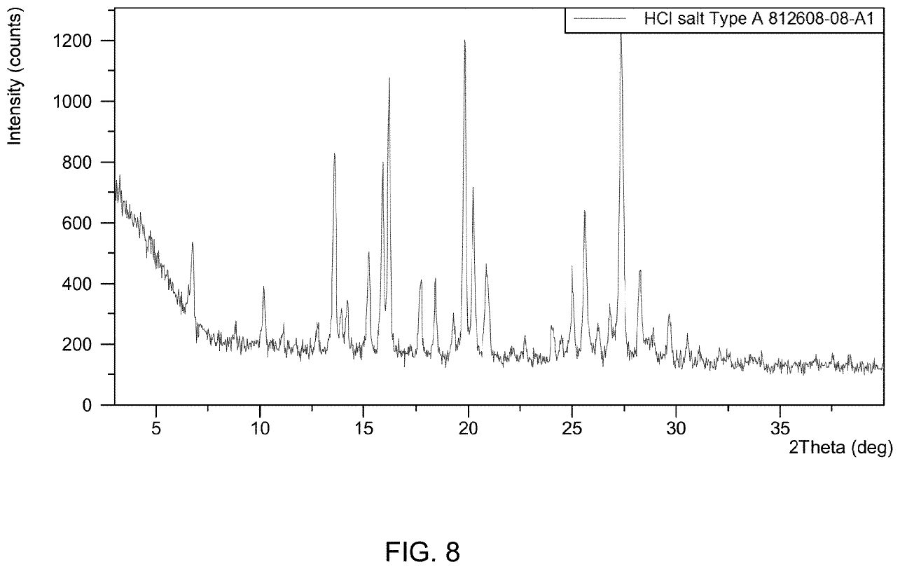

The method of any one of embodiments 85-89, wherein the form is characterized by an x-ray diffraction pattern (XRPD) substantially the same as FIG. 8.

### Embodiment 103

The method of any one of embodiments 85-102, wherein the crystalline salt form comprises Form A characterized by an endotherm at one or more of 196.2° C., 214.8° C., and 274.0° C.

### Embodiment 104

The method of any one of embodiments 85-103, wherein the crystalline salt is further characterized by a peak endotherm at one or more of 198.9° C., 218.0° C., and 275.9° C.

### Embodiment 105

The method of any one of embodiments 85-104, wherein the crystalline salt is further characterized by an onset temperature of 274.0° C.

### Embodiment 106

The method of any one of embodiments 85-105, further characterized by weight loss of 1.7% up to 150° C.

### Embodiment 107

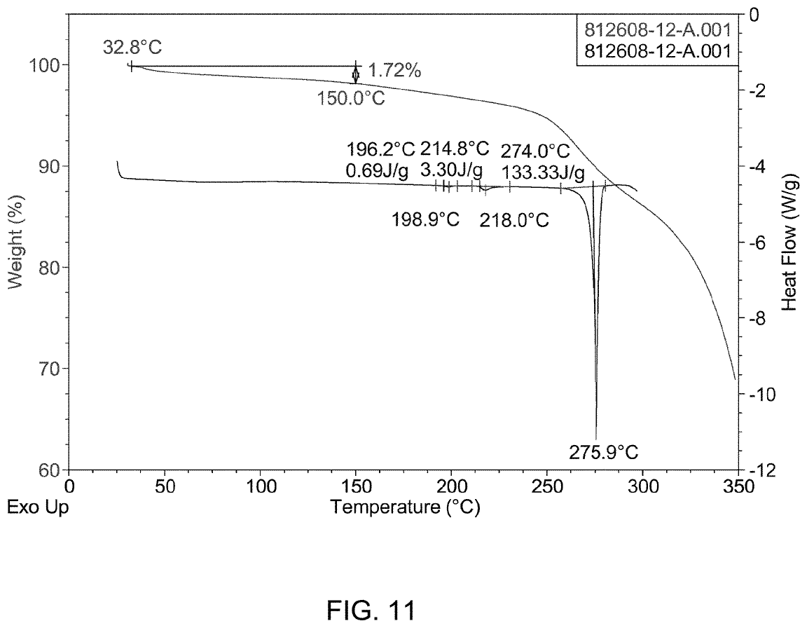

The method of any one of embodiments 85-106, hydrochloric acid salt characterized by a TGA-DSC thermogram substantially the same as FIG. 11.

Other features of the present disclosure should become apparent in the course of the above descriptions of exemplary embodiments that are given for illustration of the disclosure and are not intended to be limiting thereof.

### Definitions

For purposes of interpreting this specification, the following definitions will apply, and whenever appropriate, terms used in the singular will also include the plural. Terms used in the specification have the following meanings unless the context clearly indicates otherwise.

All methods described herein can be performed in any suitable order unless otherwise indicated herein or otherwise clearly contradicted by context. The use of any and all examples, or exemplary language (e.g. “such as”) provided herein is intended merely to better illuminate the present disclosure and does not pose a limitation on the scope of the present disclosure otherwise claimed.

The term “a,” “an,” “the” and similar terms used in the context of the present disclosure (especially in the context of the claims) are to be construed to cover both the singular and plural unless otherwise indicated herein or clearly contradicted by the context.

“Prodrug” is meant to indicate that compound of structure (I) may be converted under physiological conditions or by solvolysis to a biologically active salt described herein. Thus, the term “prodrug” refers to a precursor of the biologically active compound of structure (I) that is pharmaceutically acceptable. In some aspects, a prodrug is inactive when administered to a subject, but is converted in vivo to the active form of the compound of structure (I), for example, by hydrolysis. The prodrug compound of structure (I) often offers advantages of solubility, tissue compatibility or delayed release in a subject organism (see, e.g., Bundgard, H., Design of Prodrugs (1985), pp. 7-9, 21-24 (Elsevier, Amsterdam). A discussion of prodrugs is provided in Higuchi, T., et al., “Pro drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound of structure (I) in vivo when such prodrug is administered to a subject. Prodrugs of an active compound of structure (I), as described herein, are typically prepared by modifying functional groups present in the active compound of structure (I) in such a way that the modifications are cleaved, either in routine manipulation or in vivo, to the parent active compound of structure (I). Prodrugs include compound of structure (I) wherein a hydroxy, amino or mercapto group is bonded to any group that, when the prodrug of the active compound of structure (I) is administered to a subject, cleaves to form a free hydroxy, free amino or free mercapto group, respectively. Examples of prodrugs include acetate, formate and benzoate derivatives of a hydroxy functional group, or acetamide, formamide and benzamide derivatives of an amine functional group in the active compound of structure (I) and the like.

The disclosure herein is also meant to encompass the in vivo metabolic products of the disclosed compound of structure (I). Such products may result from, for example, the oxidation, reduction, hydrolysis, amidation, esterification, and the like of the administered compound of structure (I), primarily due to enzymatic processes. Accordingly, the disclosure includes the metabolic products of the compound of structure (I) produced by a process comprising administering a compound of structure (I) of this disclosure to a subject for a period of time sufficient to yield a metabolic product thereof. Such products are typically identified by administering a radiolabelled compound of structure (I) of the disclosure in a detectable dose to an animal, such as rat, mouse, guinea pig, monkey, or to human, allowing sufficient time for metabolism to occur, and isolating its conversion products from the urine, blood or other biological samples.

The use of the words “optional” or “optionally” means that the subsequently described event or circumstances may or may not occur, and that the description includes instances where the event or circumstance occurs and instances in which it does not. For example, “optionally substituted aryl” means that the aryl radical may or may not be substituted and that the description includes both substituted aryl radicals and aryl radicals having no substitution.

The phrase “pharmaceutically acceptable” indicates that the substance or composition must be compatible chemically and/or toxicologically, with the other ingredients comprising a formulation, and/or the mammal being treated therewith.

The terms “transforming growth factor beta receptor I kinase,” “TBR1 kinase,” “TGFβ kinase,” “activin A receptor type II-like kinase,” or “ALK5” are used interchangeably herein. The term “TGFβ type I receptor kinase (ALK5)” mediated disorder or disease” or “ALK5-mediated disorder or disease” refers to any disorder or disease which is directly or indirectly regulated by ALK5. The compound of structure (I) may be used either in free (neutral) or salt form. Both the free form and the salts of these end products are within the scope of the present disclosure. If so desired, one form of the compound may be converted into another form. A free base or acid may be converted into a salt, or a salt may be converted into the free compound or another salt.

Pharmaceutically acceptable salts are preferred. However, other salts may be useful, e.g., in isolation or purification steps which may be employed during preparation, and thus, are contemplated within the scope of the present disclosure.

As used herein, “pharmaceutically acceptable salts” refer to derivatives of the compound of structure (I) wherein the parent compound is modified by making acid or base salts thereof. For example, pharmaceutically acceptable salts include acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate/hydroxymalonate, mandelate, mesylate, methylsulphate, mucate, naphthoate, napsylate, nicotinate, nitrate, octadecanoate, oleate, oxalate, palmitate, pamoate, phenylacetate, phosphate/hydrogen phosphate/dihydrogen phosphate, polygalacturonate, propionate, salicylates, stearate, succinate, sulfamate, sulfosalicylate, tartrate, tosylate, trifluoroacetate or xinafoate salt form.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present disclosure can be synthesized from the parent compound that contains a basic or acidic moiety by conventional chemical methods. Generally, such salts can be prepared by reacting the free acid or base forms of a compound of structure (I) with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Allen, L. V., Jr., ed., Remington: The Science and Practice of Pharmacy, 22nd Edition, Pharmaceutical Press, London, UK (2012), the disclosure of which is hereby incorporated by reference.

The compound of structure (I) may be capable of forming co-crystals with suitable co-crystal formers. These co-crystals may be prepared by known co-crystal forming procedures. Such procedures include grinding, heating, co-subliming, co-melting, or contacting disclosure compound of structure (I) with the co-crystal former under crystallization conditions and isolating co-crystals thereby formed. Suitable co-crystal formers include those described in WO 2004/078163. Hence the present disclosure further provides co-crystals comprising a compound of the present disclosure.

Any formula given herein is also intended to represent unlabeled forms as well as isotopically labeled forms of the compounds. An isotopically labeled compound of structure (I) has the structure depicted by the formula given herein except that one or more atoms are replaced by an atom having a selected atomic mass or mass number. Examples of isotopes that can be incorporated into a compound of structure (I) include isotopes of hydrogen, carbon, nitrogen, oxygen, phosphorous, fluorine, chlorine and idodine, such as ²H, ³H, ¹¹C, ¹³C, ¹⁴C, ¹⁵N, ¹⁸F ³¹P, ³²P, ³⁵S, ³⁶Cl, ¹²³I, ¹²⁴I, ¹²⁵I respectively. The present disclosure includes an isotopically labeled compound of structure (I), for example those into which radioactive isotopes, such as ³H and ¹⁴C, or those into which non-radioactive isotopes, such as ²H and ¹³C are present. Such an isotopically labelled compound is useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of subjects. In particular, an ¹⁸F or labeled compound may be particularly desirable for PET or SPECT studies.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of structure (I). The concentration of such a heavier isotope, specifically deuterium, may be defined by the isotopic enrichment factor. The term “isotopic enrichment factor” as used herein means the ratio between the isotopic abundance and the natural abundance of a specified isotope.

An isotopically labeled compound of structure (I) can generally be prepared by conventional techniques known to those skilled in the art or by processes disclosed in the schemes or in the examples and preparations described below (or analogous process to those described herein), by substituting an appropriate or readily available isotopically labeled reagent for a non-isotopically labeled reagent otherwise employed. Such compounds have a variety of potential uses, e.g., as standards and reagents in determining the ability of a potential pharmaceutical compound to bind to target proteins or receptors, or for imaging compounds of this disclosure bound to biological receptors in vivo or in vitro.

As used herein, “polymorph(s)” refer to crystalline form(s) having the same chemical structure/composition but different spatial arrangements of the molecules and/or ions forming the crystals. A compound of structure (I) can be provided as amorphous solids or crystalline solids. Lyophilization can be employed to provide a compound of structure (I) as a solid.

“Treating” or “treatment” as used herein refers to the administration of a medication or medical care to a subject, such as a human, having a disease or condition of interest, e.g., a disease mediated by ALK5 such as anemia, MDS or ACD, including: (i) inhibiting or ameliorating the disease or condition, i.e., slowing or arresting its development or reducing the development of the disease or condition or at least one of the clinical symptoms thereof; (ii) relieving the disease or condition, i.e., causing regression of the disease or condition either physically, (e.g., stabilization of a discernible symptom), physiologically, (e.g., stabilization of a physical parameter), or both); (iii) relieving the symptoms resulting from the disease or condition, (e.g., pain, weight loss, cough, fatigue, weakness, etc.) without addressing the underlying disease or condition (iv) alleviating or ameliorating at least one physical parameter including those which may not be discernible by the subject; and/or (v) preventing or delaying the onset or development or progression of the disease or disorder from occurring in a subject (e.g., a mammal), in particular, when such a subject (e.g., a mammal) is predisposed to the disease or disorder but has not yet been diagnosed as having it. As used herein, the terms “disease”, “disorder” and “condition” may be used interchangeably or may be different in that the particular malady or condition may not have a known causative agent (so that etiology has not yet been confirmed) and it is therefore not yet recognized as a disease but only as an undesirable condition or syndrome, wherein a more or less specific set of symptoms have been identified by clinicians.

“Subject” includes humans, domestic animals, such as laboratory animals (e.g., dogs, monkeys, rats, mice, etc.), household pets (e.g., cats, dogs, rabbits, etc.), and livestock (e.g., pigs, cattle, sheep, goats, horses, etc.), and non-domestic animals (e.g., bears, elephants, porcupines, etc.). In embodiments, the subject is a mammal. In embodiments, a subject is a human. The term “patient” may be used interchangeably with the term “subject.”

As used herein, a subject is “in need of” a treatment if such subject would benefit biologically, medically or in quality of life from such treatment (preferably, a human).

As used herein, the term “treatment break” or “holiday” refers to the period of time between administration of a first therapeutic agent and a second therapeutic agent or may also refer to a period of time between cycles of treatment.

A treatment cycle comprises of four weeks of administration of a compound of structure (I).

The term “baseline” is used to refer to an initial measurement of a condition or parameter that is taken at an early time point and used for comparison over time to look for changes. In certain embodiments, a baseline measurement will be taken prior to treatment. In other embodiments, a baseline measurement will be after treatment has commenced, but prior to a subsequent treatment.

As used herein, the term “inhibit”, “inhibition” or “inhibiting” refers to the reduction or suppression of a given condition, symptom, or disorder, or disease, or a significant decrease in the baseline activity of a biological activity or process.

The terms “effective amount” or “therapeutically effective amount” are used interchangeably herein and refer to the amount of a compound of structure (I) or composition which, when administered to a subject, such as a human, is sufficient to effect treatment of an ALK5-mediated disease, such as MDS. The amount of a compound of structure (I) or composition that constitutes an “effective amount” will vary depending on the condition being treated and its severity, the manner of administration, the duration of treatment, and/or the age of the subject to be treated, but can be determined routinely by one of ordinary skill in the art based on his own knowledge and this disclosure. In embodiments, an “effective amount” effects treatment (e.g., treats, prevents, inhibits, relieves, promotes, improves, increases, reduces, and the like) as measured by a statistically significant change in one or more indications, symptoms, signs, diagnostic tests, vital signs, and the like. In other embodiments, an “effective amount” suppresses, manages, or prevents a condition as measured by a lack of a statistically significant change in one or more indications, symptoms, signs, diagnostic tests, vital signs, and the like.

In particular embodiments, the term “an effective amount” of a composition of the present disclosure refers to an amount of the composition of the present disclosure that will elicit the biological or medical response of a subject, for example, reduction or inhibition of an enzyme or a protein activity, or ameliorate symptoms, alleviate conditions, slow or delay disease progression, or prevent a disease, etc. In one embodiment, the term “an effective amount” refers to the amount of the composition of the present disclosure that, when administered to a subject, is effective to (1) at least partially alleviate, inhibit, prevent and/or ameliorate a condition, or a disorder or a disease mediated by ALK5; or (2) reducing or inhibiting the activity of ALK5.

In another embodiment, the term “effective amount” refers to the amount of the composition of the present disclosure that, when administered to a cell, or a tissue, or a non-cellular biological material, or a medium, is effective to at least partially reducing or inhibiting the activity of ALK5; or at least partially reducing or inhibiting the expression of ALK5.

The effective amount can vary depending on such factors as the size and weight of the subject, the type of illness, or the particular composition of the present disclosure. One of ordinary skill in the art would be able to study the factors contained herein and make the determination regarding the effective amount of the compositions of the present disclosure without undue experimentation.

The regimen of administration can affect what constitutes an effective amount. The composition of the present disclosure can be administered to the subject either prior to or after the onset of an ALK5-mediated disease, disorder or condition. Further, several divided dosages, as well as staggered dosages, can be administered daily or sequentially, or the dose can be continuously infused, or can be a bolus injection. Further, the dosages of the composition(s) of the present disclosure can be proportionally increased or decreased as indicated by the exigencies of the therapeutic or prophylactic situation.

As used herein, “statistically significant” refers to a p value of 0.050 or less when calculated using the Students t-test and indicates that it is unlikely that a particular event or result being measured has arisen by chance.

As used herein, the terms “marker”, “biomarker” and “biological marker” are used interchangeably herein to refer to a characteristic that is objectively measured and evaluated as an indicator of normal biological processes, pathogenic processes, or pharmacologic responses to a therapeutic intervention.

In the present description, any concentration range, percentage range, ratio range, or integer range is to be understood to include the value of any integer within the recited range and, when appropriate, fractions thereof (such as one tenth and one hundredth of an integer), unless other-wise indicated. Also, any number range recited herein relating to any physical feature, such as polymer subunits, size, or thickness, are to be understood to include any integer within the recited range, unless otherwise indicated. As used herein, the term “about” means±20%, ±10%, ±5% or ±1% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives.

Unless the context requires otherwise, throughout the present specification and claims, the word “comprise” and variations thereof, such as, “comprises” and “comprising,” as well as synonymous terms like “include” and “have” and variants thereof, are to be construed in an open, inclusive sense; that is, as “including, but not limited to,” such that recitation of items in a list is not to the exclusion of other like items that may also be useful in the materials, compositions, devices, and methods of this technology. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs.

### Pharmaceutical Compositions and Combinations and Dosage Regimes

The compound of structure (I), or a pharmaceutically acceptable salt or prodrug thereof, is typically used as a pharmaceutical composition (e.g., a compound of structure (I), or a pharmaceutically acceptable salt or prodrug thereof, and at least one pharmaceutically acceptable carrier).

A “pharmaceutically acceptable carrier (diluent or excipient)” refers to media generally accepted in the art for the delivery of biologically active agents to animals, in particular, mammals, including, generally recognized as safe (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, buffering agents (e.g., maleic acid, tartaric acid, lactic acid, citric acid, acetic acid, sodium bicarbonate, sodium phosphate, and the like), disintegration agents, lubricants, sweetening agents, flavoring agents, dyes, and the like and combinations thereof, as would be known to those skilled in the art (see, for example, Allen, L. V., Jr. et al., Remington: The Science and Practice of Pharmacy (2 Volumes), 22nd Edition, Pharmaceutical Press (2012).

In one aspect, the present disclosure provides a pharmaceutical composition comprising a compound of structure (I), or a pharmaceutically acceptable salt or prodrug thereof, and a pharmaceutically acceptable carrier. In a further embodiment, the composition comprises at least two pharmaceutically acceptable carriers, such as those described herein. In embodiments, a pharmaceutical composition comprises a pharmaceutically acceptable salt of a compound of structure (I). In some embodiments, a pharmaceutical composition comprises a pharmaceutically acceptable acid addition salt of a compound of structure (I). In particular embodiments, a pharmaceutical composition comprises a hydrochloric acid salt of a compound of structure (I).

For purposes of the present disclosure, unless designated otherwise, solvates and hydrates are generally considered compositions. Preferably, pharmaceutically acceptable carriers are sterile. The pharmaceutical composition can be formulated for particular routes of administration such as oral administration, parenteral administration, and rectal administration, etc. In addition, the pharmaceutical compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. Typically, the pharmaceutical compositions are tablets or gelatin capsules comprising the active ingredient together with one or more of:

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

Tablets may be either film coated or enteric coated according to methods known in the art.

Suitable compositions for oral administration include an effective amount of a compound of structure (I), or a pharmaceutically acceptable salt or prodrug thereof, in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of structure (I), or a pharmaceutically acceptable salt or prodrug thereof, with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound of structure (I), or a pharmaceutically acceptable salt or prodrug thereof, optionally with carriers, optionally a rate controlling barrier to deliver the compound to the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurized container, pump, spray, atomizer or nebulizer, with or without the use of a suitable propellant.

The present disclosure further provides anhydrous pharmaceutical compositions and dosage forms comprising a compound of structure (I), or a pharmaceutically acceptable salt or prodrug thereof, as active ingredients, since water may facilitate the degradation of certain compounds.

Anhydrous pharmaceutical compositions and dosage forms of the disclosure can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include hermetically sealed foils, plastics, unit dose containers (e.g., vials), blister packs, and strip packs.

The present disclosure further provides pharmaceutical compositions and dosage forms that comprise one or more agents that reduce the rate by which the compound of structure (I), or a pharmaceutically acceptable salt or prodrug thereof, as an active ingredient will decompose. Such agents, which are referred to herein as “stabilizers,” include antioxidants such as ascorbic acid, pH buffers, or salt buffers, etc.

The compound of structure (I), or a pharmaceutically acceptable salt or prodrug thereof, is typically formulated into pharmaceutical dosage forms to provide an easily controllable dosage of the drug and to give the subject an elegant and easily handleable product. The dosage regimen for a compound of structure (I), or a pharmaceutically acceptable salt or prodrug thereof, will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the subject, and the effect desired.

A compound of structure (I), or a pharmaceutically acceptable salt or prodrug thereof, may be administered in a single daily dose, or the total daily dosage may be administered in divided doses of two, three, or four times daily.

Due to intersubject variability in compound pharmacokinetics, individualization of dosing regimen is provided in certain embodiments. Dosing for a compound of structure (I), or a pharmaceutically acceptable salt or prodrug thereof can be found by routine experimentation in light of the instant disclosure and/or can be derived by one of ordinary skill in the art.

The effective amount or dose of the compound of structure (I), or a pharmaceutically acceptable salt or prodrug thereof, can be estimated initially from cell culture assays. Then, the dosage can be formulated for use in animal models so as to achieve a circulating concentration range that includes the IC50 as determined in cell culture (i.e., the concentration of the test compound which achieves a half-maximal inhibition of the protein kinase activity). Such information can then be used to more accurately determine useful doses in humans.

Toxicity and therapeutic efficacy of the compounds described herein can be determined by standard pharmaceutical procedures in cell cultures or experimental animals, e.g., by determining the IC50 and the LD50 (both of which are discussed elsewhere herein) for a subject compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See, e.g., GOODMAN & GILMAN'S THE PHARMACOLOGICAL BASIS OF THERAPEUTICS, Ch. 3, 9th ed., Ed. by Hardman, J., and Limbard, L., McGraw-Hill, New York City, 1996, p. 46.)

Dosage intervals can also be determined using MEC value. In some embodiments, the compound of structure (I) is administered using a regimen that maintains plasma levels above the MEC for 10-90% of the time, preferably between 30-90% and most preferably between 50-90%. Dosage amount and interval may be adjusted individually to provide plasma levels of the active species which are sufficient to maintain desired pharmacological effects. Dosages necessary to achieve the MEC will depend on individual characteristics and route of administration. HPLC assays or bioassays can be used to determine plasma concentrations.

In some embodiments of a method of the present disclosure, the compound of structure (I) is administered as a maintenance dosage regime. In some embodiments, maintenance dose is the dose at which the subject achieves and maintains for a period of time a predetermined threshold level of a biomarker, e.g., hemoglobin or hepcidin, wherein the period of time is 1 week, 2 weeks 3 weeks, 4 weeks, 6 weeks, 8 weeks, 10 weeks, 3 months, 4 months, or longer. In some embodiments the maintenance dosage regime comprises a daily dosage, a twice weekly dosage, a weekly dosage, or a dosage every two weeks. In certain embodiments, the maintenance dose is less than a maximum tolerated dose. In other embodiments, the maintenance dose is less than a maximum administered dose.

For example, in certain embodiments, effective amounts of the compound of structure (I) range from approximately 0.1 mg/m² to 10,500 mg/m² per week. Additional illustrative amounts range from 0.1 mg to 3000 mg, 1 mg to 1000 mg, 2 mg to 500 mg, 1 mg to 2000 mg, 1 mg to 1000 mg, 1 mg to 300 mg, 1 mg to 100 mg, 1 mg to 90 mg, 1 mg to 80 mg, 1 mg to 70 mg, 1 mg to 60 mg, 20 mg to 50 mg, 1 mg to 40 mg, 1 mg to 30 mg, 1 mg to 20 mg, 1 mg to 10 mg, 1 mg to 3 mg, 3 mg to 2000 mg, 3 mg to 1000 mg, 3 mg to 300 mg, 3 mg to 100 mg, 3 mg to 90 mg, 3 mg to 80 mg, 3 mg to 70 mg, 3 mg to 60 mg, 20 mg to 50 mg, 3 mg to 40 mg, 3 mg to 30 mg, 3 mg to 10 mg, 10 mg to 2000 mg, 10 mg to 1000 mg, 10 mg to 300 mg, 10 mg to 150 mg, 10 mg to 100 mg, 10 mg to 90 mg, 10 mg to 80 mg, 10 mg to 70 mg, 10 mg to 60 mg, 10 mg to 50 mg, 10 mg to 40 mg, 10 mg to 30 mg, 10 mg to 20 mg, 20 mg to 2000 mg, 20 mg to 1000 mg, 20 mg to 200 mg, 20 mg to 100 mg, 20 mg to 90 mg, 20 mg to 85 mg, 20 mg to 80 mg, 20 mg to 75 mg, 20 mg to 70 mg, 20 mg to 65 mg, 20 mg to 60 mg, 20 mg to 55 mg, 20 mg to 50 mg, 20 mg to 45 mg, 20 mg to 40 mg, 20 mg to 35 mg, 20 mg to 30 mg, 20 mg to 25 mg, 30 mg to 2000 mg, 30 mg to 1000 mg, 30 mg to 300 mg, 30 mg to 100 mg, 30 mg to 95 mg, 30 mg to 90 mg, 30 mg to 95 mg, 30 mg to 80 mg, 30 mg to 75 mg, 30 mg to 70 mg, 30 mg to 65 mg, 30 mg to 60 mg, 30 mg to 55 mg, 30 mg to 50 mg, 30 mg to 45 mg, 30 mg to 40 mg, 30 mg to 35 mg, 35 mg to 2000 mg, 35 mg to 1000 mg, 35 mg to 400 mg, 35 mg to 100 mg, 35 mg to 90 mg, 35 mg to 85 mg, 35 mg to 80 mg, 35 mg to 75 mg, 35 mg to 70 mg, 35 mg to 65 mg, 35 mg to 60 mg, 35 mg to 55 mg, 35 mg to 50 mg, 35 mg to 45 mg, 35 mg to 40 mg, 40 mg to 2000 mg, 40 mg to 1000 mg, 40 mg to 400 mg, 40 mg to 100 mg, 40 mg to 90 mg, 40 mg to 85 mg, 40 mg to 80 mg, 40 mg to 75 mg, 40 mg to 70 mg, 40 mg to 65 mg, 40 mg to 60 mg, 40 mg to 55 mg, 40 mg to 50 mg, 40 mg to 45 mg, 45 mg to 2000 mg, 45 mg to 1000 mg, 45 mg to 450 mg, 45 mg to 100 mg, 45 mg to 90 mg, 45 mg to 85 mg, 45 mg to 80 mg, 45 mg to 75 mg, 45 mg to 70 mg, 45 mg to 65 mg, 45 mg to 60 mg, 45 mg to 55 mg, 45 mg to 50 mg, 50 mg to 2000 mg, 50 mg to 1000 mg, 50 mg to 500 mg, 50 mg to 100 mg, 50 mg to 90 mg, 50 mg to 85 mg, 50 mg to 80 mg, 50 mg to 75 mg, 50 mg to 70 mg, 50 mg to 65 mg, 50 mg to 60 mg, 50 mg to 55 mg, 55 mg to 2000 mg, 55 mg to 1000 mg, 55 mg to 550 mg, 55 mg to 100 mg, 55 mg to 90 mg, 55 mg to 85 mg, 55 mg to 80 mg, 55 mg to 75 mg, 55 mg to 70 mg, 55 mg to 65 mg, 55 mg to 60 mg, 60 mg to 2000 mg, 60 mg to 1000 mg, 60 mg to 600 mg, 60 mg to 100 mg, 60 mg to 90 mg, 60 mg to 85 mg, 60 mg to 80 mg, 60 mg to 75 mg, 60 mg to 70 mg, 60 mg to 65 mg, 65 mg to 2000 mg, 65 mg to 1000 mg, 65 mg to 300 mg, 65 mg to 100 mg, 65 mg to 90 mg, 65 mg to 85 mg, 65 mg to 80 mg, 65 mg to 75 mg, 65 mg to 70 mg, 70 mg to 2000 mg, 70 mg to 1000 mg, 70 mg to 300 mg, 70 mg to 100 mg, 70 mg to 90 mg, 70 mg to 85 mg, 70 mg to 80 mg, 70 mg to 75 mg, 75 mg to 2000 mg, 75 mg to 1000 mg, 75 mg to 300 mg, 75 mg to 100 mg, 75 mg to 90 mg, 75 mg to 85 mg, 75 mg to 80 mg, 80 mg to 2000 mg, 80 mg to 1000 mg, 80 mg to 800 mg, 80 mg to 100 mg, 80 mg to 90 mg, 80 mg to 85 mg, 85 mg to 2000 mg, 85 mg to 1000 mg, 85 mg to 300 mg, 85 mg to 100 mg, 85 mg to 90 mg, 90 mg to 2000 mg, 90 mg to 1000 mg, 90 mg to 300 mg, 90 mg to 100 mg, 90 mg to 95 mg, 95 mg to 2000 mg, 95 mg to 1000 mg, 95 mg to 300 mg, 95 mg to 100 mg, 100 mg to 2000 mg, 100 mg to 1000 mg, 100 mg to 300 mg, 300 mg to 2000 mg, 300 mg to 1000 mg or 1000 mg to 2000 mg

In certain embodiments, an effective amount ranges from approximately 2.5 mg/m² to 1500 mg/m² per day. In certain embodiments, the daily dosage is from 10 to 350 mg, from 90 to 120 mg, preferably 20 mg, 40 mg, 60 mg, 90 mg, 120 mg, 160 mg, 210 mg or 270 mg.

In some embodiments, the concentration the compound of structure (I) provided in the pharmaceutical compositions is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of the compound of structure (I) provided in the pharmaceutical compositions is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of the compound of structure (I) provided in the pharmaceutical compositions is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% w/w, w/v or v/v.

In some embodiments, the concentration of the compound of structure (I) provided in the pharmaceutical compositions is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v.

In cases of local administration or selective uptake, the effective local concentration of the drug may not be related to plasma concentration, and other procedures known in the art may be employed to determine the correct dosage amount and interval.

Certain methods disclosed herein serve to modify a regimen of treatment for a subject in need thereof. That is, this disclosure provides methods for modifying treatment regimens as well as methods of treatment themselves.

Expression of a biomarker may be determined in a sample collected from a subject, (e.g., blood plasma, serum or bone marrow aspirate), prior to treatment, during treatment and after treatment. In such embodiments, the expression levels prior to treatment or during treatment, prior to a subsequent administration step, may be used to determine changes in expression levels used to modify dosage amounts, such as increasing or decreasing loading dosages and increasing or decreasing maintenance dosages and also to confirm the efficacy of a treatment.

In additional embodiments, the methods according to the present disclosure include administering a loading dose. In some embodiments, a subsequent loading dose is administered. In some embodiments, 1, 2, 3 or 4 loading doses or more are administered before a maintenance dosage regime is initiated.

In some embodiments, a method of treating an ALK5-mediated disease according to the present disclosure comprises:
a) administering a loading dose of a compound of structure (I):

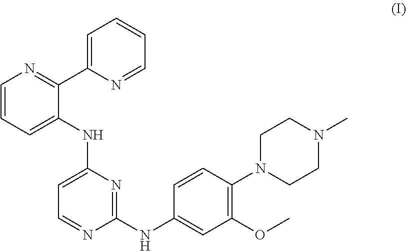 [SMILES: COc1cc(Nc2nccc(Nc3cccnc3-c3ccccn3)n2)ccc1N1CCN(C)CC1]

or a pharmaceutically acceptable salt, or prodrug thereof, to the subject; and
b) determining if a hemoglobin level is above, at, or below a predetermined loading dose threshold or determining if a change in hemoglobin level is above, at, or below a predetermined amount, wherein:
   i) if the hemoglobin level is below the predetermined loading dose threshold or if the change in hemoglobin level is below the predetermined amount, then administering a subsequent loading dose and repeating steps a-b; or
   ii) if the hemoglobin level is at or above the predetermined loading dose threshold or if the change in hemoglobin level is at or above the predetermined amount, then administering the compound of structure (I) according to the maintenance dosage regime.

In some embodiments, a method according to the present disclosure comprises:
a) administering a loading dose of the compound of structure (I) to the subject, and
b) determining if a hemoglobin level is above, at, or below a predetermined loading dose threshold, wherein:
   i) if the hemoglobin level is below a predetermined loading dose threshold, then administering a subsequent loading dose and repeating steps a-b; or
   ii) if the hemoglobin level is at or above the predetermined loading dose threshold, then administering the compound of structure (I) according to the maintenance dosage regime.

In some embodiments, a method according to the present disclosure comprises:
a) administering a loading dose of the compound of structure (I) to the subject, and
b) determining if a change in hemoglobin level is above, at, or below a predetermined amount, wherein:
   i) if the change in hemoglobin level is below the predetermined amount, then administering a subsequent loading dose and repeating steps a-b; or
   ii) if the change in hemoglobin level is at or above the predetermined amount, then administering the compound of structure (I) according to the maintenance dosage regime.

In certain embodiments of the methods according to the present disclosure, the change in hemoglobin level is determined from a baseline level of hemoglobin, i.e., before administration of a compound of structure (I). In other embodiments, the change in hemoglobin level is determined from a previous level of hemoglobin, e.g., after administration of a previous loading dose.

In certain embodiments, the loading dose is from 0.1 mg to 3000 mg, 1 mg to 1000 mg, 2 mg to 500 mg, 1 mg to 2000 mg, 1 mg to 1000 mg, 1 mg to 300 mg, 1 mg to 100 mg, 1 mg to 90 mg, 1 mg to 80 mg, 1 mg to 70 mg, 1 mg to 60 mg, 20 mg to 50 mg, 1 mg to 40 mg, 1 mg to 30 mg, 1 mg to 20 mg, 1 mg to 10 mg, 1 mg to 3 mg, 3 mg to 2000 mg, 3 mg to 1000 mg, 3 mg to 300 mg, 3 mg to 100 mg, 3 mg to 90 mg, 3 mg to 80 mg, 3 mg to 70 mg, 3 mg to 60 mg, 20 mg to 50 mg, 3 mg to 40 mg, 3 mg to 30 mg, 3 mg to 10 mg, 10 mg to 2000 mg, 10 mg to 1000 mg, 10 mg to 300 mg, 10 mg to 150 mg, 10 mg to 100 mg, 10 mg to 90 mg, 10 mg to 80 mg, 10 mg to 70 mg, 10 mg to 60 mg, 10 mg to 50 mg, 10 mg to 40 mg, 10 mg to 30 mg, 10 mg to 20 mg, 20 mg to 2000 mg, 20 mg to 1000 mg, 20 mg to 200 mg, 20 mg to 100 mg, 20 mg to 90 mg, 20 mg to 85 mg, 20 mg to 80 mg, 20 mg to 75 mg, 20 mg to 70 mg, 20 mg to 65 mg, 20 mg to 60 mg, 20 mg to 55 mg, 20 mg to 50 mg, 20 mg to 45 mg, 20 mg to 40 mg, 20 mg to 35 mg, 20 mg to 30 mg, 20 mg to 25 mg, 30 mg to 2000 mg, 30 mg to 1000 mg, 30 mg to 300 mg, 30 mg to 100 mg, 30 mg to 95 mg, 30 mg to 90 mg, 30 mg to 95 mg, 30 mg to 80 mg, 30 mg to 75 mg, 30 mg to 70 mg, 30 mg to 65 mg, 30 mg to 60 mg, 30 mg to 55 mg, 30 mg to 50 mg, 30 mg to 45 mg, 30 mg to 40 mg, 30 mg to 35 mg, 35 mg to 2000 mg, 35 mg to 1000 mg, 35 mg to 400 mg, 35 mg to 100 mg, 35 mg to 90 mg, 35 mg to 85 mg, 35 mg to 80 mg, 35 mg to 75 mg, 35 mg to 70 mg, 35 mg to 65 mg, 35 mg to 60 mg, 35 mg to 55 mg, 35 mg to 50 mg, 35 mg to 45 mg, 35 mg to 40 mg, 40 mg to 2000 mg, 40 mg to 1000 mg, 40 mg to 400 mg, 40 mg to 100 mg, 40 mg to 90 mg, 40 mg to 85 mg, 40 mg to 80 mg, 40 mg to 75 mg, 40 mg to 70 mg, 40 mg to 65 mg, 40 mg to 60 mg, 40 mg to 55 mg, 40 mg to 50 mg, 40 mg to 45 mg, 45 mg to 2000 mg, 45 mg to 1000 mg, 45 mg to 450 mg, 45 mg to 100 mg, 45 mg to 90 mg, 45 mg to 85 mg, 45 mg to 80 mg, 45 mg to 75 mg, 45 mg to 70 mg, 45 mg to 65 mg, 45 mg to 60 mg, 45 mg to 55 mg, 45 mg to 50 mg, 50 mg to 2000 mg, 50 mg to 1000 mg, 50 mg to 500 mg, 50 mg to 100 mg, 50 mg to 90 mg, 50 mg to 85 mg, 50 mg to 80 mg, 50 mg to 75 mg, 50 mg to 70 mg, 50 mg to 65 mg, 50 mg to 60 mg, 50 mg to 55 mg, 55 mg to 2000 mg, 55 mg to 1000 mg, 55 mg to 550 mg, 55 mg to 100 mg, 55 mg to 90 mg, 55 mg to 85 mg, 55 mg to 80 mg, 55 mg to 75 mg, 55 mg to 70 mg, 55 mg to 65 mg, 55 mg to 60 mg, 60 mg to 2000 mg, 60 mg to 1000 mg, 60 mg to 600 mg, 60 mg to 100 mg, 60 mg to 90 mg, 60 mg to 85 mg, 60 mg to 80 mg, 60 mg to 75 mg, 60 mg to 70 mg, 60 mg to 65 mg, 65 mg to 2000 mg, 65 mg to 1000 mg, 65 mg to 300 mg, 65 mg to 100 mg, 65 mg to 90 mg, 65 mg to 85 mg, 65 mg to 80 mg, 65 mg to 75 mg, 65 mg to 70 mg, 70 mg to 2000 mg, 70 mg to 1000 mg, 70 mg to 300 mg, 70 mg to 100 mg, 70 mg to 90 mg, 70 mg to 85 mg, 70 mg to 80 mg, 70 mg to 75 mg, 75 mg to 2000 mg, 75 mg to 1000 mg, 75 mg to 300 mg, 75 mg to 100 mg, 75 mg to 90 mg, 75 mg to 85 mg, 75 mg to 80 mg, 80 mg to 2000 mg, 80 mg to 1000 mg, 80 mg to 800 mg, 80 mg to 100 mg, 80 mg to 90 mg, 80 mg to 85 mg, 85 mg to 2000 mg, 85 mg to 1000 mg, 85 mg to 300 mg, 85 mg to 100 mg, 85 mg to 90 mg, 90 mg to 2000 mg, 90 mg to 1000 mg, 90 mg to 300 mg, 90 mg to 100 mg, 90 mg to 95 mg, 95 mg to 2000 mg, 95 mg to 1000 mg, 95 mg to 300 mg, 95 mg to 100 mg, 100 mg to 2000 mg, 100 mg to 1000 mg, 100 mg to 300 mg, 300 mg to 2000 mg, 300 mg to 1000 mg or 1000 mg to 2000 mg, or a range defined by any two of these amounts.

In certain embodiments, the loading dose is from 10 mg to 300 mg, 30 mg to 100 mg, or selected from 10 mg, 15 mg, 20 mg, 30 mg, 35 mg, 40 mg, 45 mg, 50 mg, 55 mg, 60 mg, 65 mg, 70 mg, 75 mg, 80 mg, 85 mg, 90 mg, 95 mg, or a range defined by any two of these amounts.

In certain embodiments of the methods of the present disclosure, the determining step d) further comprises a step of measuring a hemoglobin level. In some embodiments, the measuring step comprises obtaining a biological sample from the subject wherein the biological sample is whole blood, serum or plasma. In certain embodiments, the biological sample is serum.

In certain embodiments, the predetermined loading dose threshold of hemoglobin is 1 g/dL, 1.5 g/dL, 2 g/dL, 2.5 g/dL, 3 g/dL, 3.5 g/dL, 4 g/dL, 4.5 g/dL, 5 g/dL, 5.5 g/dL, 6 g/dL, 6.5 g/dL, 7 g/dL, 7.5 g/dL, 8 g/dL, 8.5 g/dL, 9 g/dL, 9.5 g/dL, 10 g/dL, 10.5 g/dL, or higher.

In certain embodiments, the hemoglobin level is below a predetermined loading dose threshold, and the method includes a step of administering a subsequent loading dose and repeating steps a-b. In further embodiments, the subsequent loading dose is the same amount as the initial loading dose. In alternative embodiments, the subsequent loading dose is increased by 1%, 2%, 5%, 10%, 15% 20%, 25% 30%, 35%, 45%, 50%, 55%, 60%, 70%, 75%, 80%, 85%, 95%, 100%, 200% or 300% compared to the loading dose administered in previous step a. In alternative embodiments, the subsequent loading dose is increased by 5 mg, 10 mg or 15 mg.

In certain embodiments, the predetermined amount of the change in hemoglobin is 0.1 g/dL, 0.2 g/dL, 0.3 g/dL, 0.4 g/dL, 0.5 g/dL, 0.6 g/dL, 0.7 g/dL, 0.8 g/dL, 0.9 g/dL, 1.0 g/dL, 1.1 g/dL, 1.2 g/dL, 1.3 g/dL, 1.4 g/dL, 1.5 g/dL, 1.6 g/dL, 1.7 g/dL, 1.8 g/dL, 1.9 g/dL, 2.0 g/dL, 2.1 g/dL, 2.2 g/dL, 2.3 g/dL, 2.4 g/dL, 2.5 g/dL, 2.6 g/dL, 2.7 g/dL, 2.8 g/dL, 2.9 g/dL, 3.0 g/dL, 3.1 g/dL, 3.2 g/dL, 3.3 g/dL, 3.4 g/dL, 3.5 g/dL, 3.6 g/dL, 3.7 g/dL, 3.8 g/dL, 3.9 g/dL, 4.0 g/dL, 4.1 g/dL, 4.2 g/dL, 4.3 g/dL, 4.4 g/dL, 4.5 g/dL, 4.6 g/dL, 4.7 g/dL, 4.8 g/dL, 4.9 g/dL, 5.0 g/dL, 5.1 g/dL, 5.2 g/dL, 5.3 g/dL, 5.4 g/dL, 5.5 g/dL, 5.6 g/dL, 5.7 g/dL, 5.8 g/dL, 5.9 g/dL, 6.0 g/dL, 6.1 g/dL, 6.2 g/dL, 6.3 g/dL, 6.4 g/dL, 6.5 g/dL, 6.6 g/dL, 6.7 g/dL, 6.8 g/dL, 6.9 g/dL, 7.0 g/dL, 7.1 g/dL, 7.2 g/dL, 7.3 g/dL, 7.4 g/dL, 7.5 g/dL, 7.6 g/dL, 7.7 g/dL, 7.8 g/dL, 7.9 g/dL, 8.0 g/dL, 8.1 g/dL, 8.2 g/dL, 8.3 g/dL, 8.4 g/dL, 8.5 g/dL, 8.6 g/dL, 8.7 g/dL, 8.8 g/dL, 8.9 g/dL, 9.0 g/dL, 9.1 g/dL, 9.2 g/dL, 9.3 g/dL, 9.4 g/dL, 9.5 g/dL, 9.6 g/dL, 9.7 g/dL, 9.8 g/dL, 9.9 g/dL, 10.0 g/dL, or higher.

In certain embodiments, the change in hemoglobin is measured from baseline, wherein the baseline level of hemoglobin is determined prior to administration of a compound of structure (I).

In certain embodiments, the change in hemoglobin level is below a predetermined amount, and the method includes a step of administering a subsequent loading dose and repeating steps a-b. In further embodiments, the subsequent loading dose is the same amount as the initial loading dose. In alternative embodiments, the subsequent loading dose is increased by 1%, 2%, 5%, 10%, 15% 20%, 25% 30%, 35%, 45%, 50%, 55%, 60%, 70%, 75%, 80%, 85%, 95%, 100%, 200% or 300% compared to the loading dose administered in step a. In alternative embodiments, the subsequent loading dose is increased by 5 mg, 10 mg or 15 mg.

In certain embodiments, the hemoglobin level is at or above the loading dose threshold and the compound of structure (I) is administered according to a maintenance dosage regime as described herein.

In certain embodiments, the maintenance dosage regime comprises administering a maintenance dosage. In certain embodiments, the maintenance dose is from 0.1 mg to 3000 mg, 1 mg to 1000 mg, 2 mg to 500 mg, 1 mg to 2000 mg, 1 mg to 1000 mg, 1 mg to 300 mg, 1 mg to 100 mg, 1 mg to 90 mg, 1 mg to 80 mg, 1 mg to 70 mg, 1 mg to 60 mg, 20 mg to 50 mg, 1 mg to 40 mg, 1 mg to 30 mg, 1 mg to 20 mg, 1 mg to 10 mg, 1 mg to 3 mg, 3 mg to 2000 mg, 3 mg to 1000 mg, 3 mg to 300 mg, 3 mg to 100 mg, 3 mg to 90 mg, 3 mg to 80 mg, 3 mg to 70 mg, 3 mg to 60 mg, 20 mg to 50 mg, 3 mg to 40 mg, 3 mg to 30 mg, 3 mg to 10 mg, 10 mg to 2000 mg, 10 mg to 1000 mg, 10 mg to 300 mg, 10 mg to 150 mg, 10 mg to 100 mg, 10 mg to 90 mg, 10 mg to 80 mg, 10 mg to 70 mg, 10 mg to 60 mg, 10 mg to 50 mg, 10 mg to 40 mg, 10 mg to 30 mg, 10 mg to 20 mg, 20 mg to 2000 mg, 20 mg to 1000 mg, 20 mg to 200 mg, 20 mg to 100 mg, 20 mg to 90 mg, 20 mg to 85 mg, 20 mg to 80 mg, 20 mg to 75 mg, 20 mg to 70 mg, 20 mg to 65 mg, 20 mg to 60 mg, 20 mg to 55 mg, 20 mg to 50 mg, 20 mg to 45 mg, 20 mg to 40 mg, 20 mg to 35 mg, 20 mg to 30 mg, 20 mg to 25 mg, 30 mg to 2000 mg, 30 mg to 1000 mg, 30 mg to 300 mg, 30 mg to 100 mg, 30 mg to 95 mg, 30 mg to 90 mg, 30 mg to 95 mg, 30 mg to 80 mg, 30 mg to 75 mg, 30 mg to 70 mg, 30 mg to 65 mg, 30 mg to 60 mg, 30 mg to 55 mg, 30 mg to 50 mg, 30 mg to 45 mg, 30 mg to 40 mg, 30 mg to 35 mg, 35 mg to 2000 mg, 35 mg to 1000 mg, 35 mg to 400 mg, 35 mg to 100 mg, 35 mg to 90 mg, 35 mg to 85 mg, 35 mg to 80 mg, 35 mg to 75 mg, 35 mg to 70 mg, 35 mg to 65 mg, 35 mg to 60 mg, 35 mg to 55 mg, 35 mg to 50 mg, 35 mg to 45 mg, 35 mg to 40 mg, 40 mg to 2000 mg, 40 mg to 1000 mg, 40 mg to 400 mg, 40 mg to 100 mg, 40 mg to 90 mg, 40 mg to 85 mg, 40 mg to 80 mg, 40 mg to 75 mg, 40 mg to 70 mg, 40 mg to 65 mg, 40 mg to 60 mg, 40 mg to 55 mg, 40 mg to 50 mg, 40 mg to 45 mg, 45 mg to 2000 mg, 45 mg to 1000 mg, 45 mg to 450 mg, 45 mg to 100 mg, 45 mg to 90 mg, 45 mg to 85 mg, 45 mg to 80 mg, 45 mg to 75 mg, 45 mg to 70 mg, 45 mg to 65 mg, 45 mg to 60 mg, 45 mg to 55 mg, 45 mg to 50 mg, 50 mg to 2000 mg, 50 mg to 1000 mg, 50 mg to 500 mg, 50 mg to 100 mg, 50 mg to 90 mg, 50 mg to 85 mg, 50 mg to 80 mg, 50 mg to 75 mg, 50 mg to 70 mg, 50 mg to 65 mg, 50 mg to 60 mg, 50 mg to 55 mg, 55 mg to 2000 mg, 55 mg to 1000 mg, 55 mg to 550 mg, 55 mg to 100 mg, 55 mg to 90 mg, 55 mg to 85 mg, 55 mg to 80 mg, 55 mg to 75 mg, 55 mg to 70 mg, 55 mg to 65 mg, 55 mg to 60 mg, 60 mg to 2000 mg, 60 mg to 1000 mg, 60 mg to 600 mg, 60 mg to 100 mg, 60 mg to 90 mg, 60 mg to 85 mg, 60 mg to 80 mg, 60 mg to 75 mg, 60 mg to 70 mg, 60 mg to 65 mg, 65 mg to 2000 mg, 65 mg to 1000 mg, 65 mg to 300 mg, 65 mg to 100 mg, 65 mg to 90 mg, 65 mg to 85 mg, 65 mg to 80 mg, 65 mg to 75 mg, 65 mg to 70 mg, 70 mg to 2000 mg, 70 mg to 1000 mg, 70 mg to 300 mg, 70 mg to 100 mg, 70 mg to 90 mg, 70 mg to 85 mg, 70 mg to 80 mg, 70 mg to 75 mg, 75 mg to 2000 mg, 75 mg to 1000 mg, 75 mg to 300 mg, 75 mg to 100 mg, 75 mg to 90 mg, 75 mg to 85 mg, 75 mg to 80 mg, 80 mg to 2000 mg, 80 mg to 1000 mg, 80 mg to 800 mg, 80 mg to 100 mg, 80 mg to 90 mg, 80 mg to 85 mg, 85 mg to 2000 mg, 85 mg to 1000 mg, 85 mg to 300 mg, 85 mg to 100 mg, 85 mg to 90 mg, 90 mg to 2000 mg, 90 mg to 1000 mg, 90 mg to 300 mg, 90 mg to 100 mg, 90 mg to 95 mg, 95 mg to 2000 mg, 95 mg to 1000 mg, 95 mg to 300 mg, 95 mg to 100 mg, 100 mg to 2000 mg, 100 mg to 1000 mg, 100 mg to 300 mg, 300 mg to 2000 mg, 300 mg to 1000 mg or 1000 mg to 2000 mg or a range defined by any two of these amounts.

In certain embodiments of the methods according to the present disclosure, the maintenance dosage regime comprises administering a maintenance dosage. In certain embodiments, the maintenance dose is from 10 mg to 300 mg, 10 mg to 150 mg, 10 mg to 100 mg, 10 mg to 90 mg, 10 mg to 80 mg, 10 mg to 70 mg, 10 mg to 60 mg, 10 mg to 50 mg, 10 mg to 40 mg, 10 mg to 30 mg, 10 mg to 20 mg, 20 mg to 300 mg, 20 mg to 200 mg, 20 mg to 100 mg, 20 mg to 90 mg, 20 mg to 85 mg, 20 mg to 80 mg, 20 mg to 75 mg, 20 mg to 70 mg, 20 mg to 65 mg, 20 mg to 60 mg, 20 mg to 55 mg, 20 mg to 50 mg, 20 mg to 45 mg, 20 mg to 40 mg, 20 mg to 35 mg, 20 mg to 30 mg, 20 mg to 25 mg, 30 mg to 300 mg, 30 mg to 100 mg, 30 mg to 95 mg, 30 mg to 90 mg, 30 mg to 95 mg, 30 mg to 80 mg, 30 mg to 75 mg, 30 mg to 70 mg, 30 mg to 65 mg, 30 mg to 60 mg, 30 mg to 55 mg, 30 mg to 50 mg, 30 mg to 45 mg, 30 mg to 40 mg, 30 mg to 35 mg, 35 mg to 300 mg, 35 mg to 100 mg, 35 mg to 90 mg, 35 mg to 85 mg, 35 mg to 80 mg, 35 mg to 75 mg, 35 mg to 70 mg, 35 mg to 65 mg, 35 mg to 60 mg, 35 mg to 55 mg, 35 mg to 50 mg, 35 mg to 45 mg, 35 mg to 40 mg, 40 mg to 300 mg, 40 mg to 100 mg, 40 mg to 90 mg, 40 mg to 85 mg, 40 mg to 80 mg, 40 mg to 75 mg, 40 mg to 70 mg, 40 mg to 65 mg, 40 mg to 60 mg, 40 mg to 55 mg, 40 mg to 50 mg, 40 mg to 45 mg, 45 mg to 300 mg, 45 mg to 100 mg, 45 mg to 90 mg, 45 mg to 85 mg, 45 mg to 80 mg, 45 mg to 75 mg, 45 mg to 70 mg, 45 mg to 65 mg, 45 mg to 60 mg, 45 mg to 55 mg, 45 mg to 50 mg, 50 mg to 300 mg, 50 mg to 100 mg, 50 mg to 90 mg, 50 mg to 85 mg, 50 mg to 80 mg, 50 mg to 75 mg, 50 mg to 70 mg, 50 mg to 65 mg, 50 mg to 60 mg, 50 mg to 55 mg, 55 mg to 300 mg, 55 mg to 100 mg, 55 mg to 90 mg, 55 mg to 85 mg, 55 mg to 80 mg, 55 mg to 75 mg, 55 mg to 70 mg, 55 mg to 65 mg, 55 mg to 60 mg, 60 mg to 300 mg, 60 mg to 100 mg, 60 mg to 90 mg, 60 mg to 85 mg, 60 mg to 80 mg, 60 mg to 75 mg, 60 mg to 70 mg, 60 mg to 65 mg, 65 mg to 300 mg, 65 mg to 100 mg, 65 mg to 90 mg, 65 mg to 85 mg, 65 mg to 80 mg, 65 mg to 75 mg, 65 mg to 70 mg, 70 mg to 300 mg, 70 mg to 100 mg, 70 mg to 90 mg, 70 mg to 85 mg, 70 mg to 80 mg, 70 mg to 75 mg, 75 mg to 300 mg, 75 mg to 100 mg, 75 mg to 90 mg, 75 mg to 85 mg, 75 mg to 80 mg, 80 mg to 300 mg, 80 mg to 100 mg, 80 mg to 90 mg, 80 mg to 85 mg, 85 mg to 300 mg, 85 mg to 100 mg, 85 mg to 90 mg, 90 mg to 300 mg, 90 mg to 100 mg, 90 mg to 95 mg, 95 mg to 300 mg, 95 mg to 100 mg, 100 mg to 300 mg, 100 mg to 150 mg, or 150 mg to 300 mg.

In additional embodiments, the methods according to the present disclosure include a maintenance dosage reduction regime. In certain embodiments, a method is provided further comprising the steps of:
c) administering the maintenance dose;
d) determining if a hemoglobin level is above, at, or below a predetermined maintenance dose threshold or determining if a change in hemoglobin level is above, at, or below a predetermined amount, wherein:
i) if the hemoglobin level is below the predetermined maintenance dose threshold or if the change in hemoglobin level is below the predetermined amount, then administering a subsequent maintenance dose and repeating steps c-d; or
ii) if the hemoglobin level is at or above the predetermined maintenance dose threshold or if the change in hemoglobin level is at or above the predetermined amount, then administering a reduced maintenance dose wherein the dosage is reduced by a predetermined amount, and optionally repeating steps c-d.

In additional embodiments, the methods according to the present disclosure include a maintenance dosage reduction regime. In certain embodiments, a method is provided further comprising the steps of:
c) administering the maintenance dose;
d) determining if a change in hemoglobin level is above, at, or below a predetermined amount, wherein:
i) if the change in hemoglobin level is below the predetermined amount, then administering a subsequent maintenance dose and repeating steps c-d; or
ii) if the change in hemoglobin level is at or above the predetermined amount, then administering a reduced maintenance dose wherein the dosage is reduced by a predetermined amount, and optionally repeating steps c-d.

In additional embodiments, the methods according to the present disclosure include a maintenance dosage reduction regime. In certain embodiments, a method is provided comprising the steps of:
c) administering a maintenance dose; and
d) determining if a hemoglobin level is above, at, or below a predetermined maintenance dose threshold, wherein:
   i) if the hemoglobin level is below a predetermined maintenance dose threshold, then administering a subsequent maintenance dose and repeating steps c-d; or
   ii) if the hemoglobin level is at or above the predetermined maintenance dose threshold, then administering a reduced maintenance dose wherein the dosage is reduced by a predetermined amount, and optionally repeating steps d-e.

In certain embodiments, determining step d) further comprises a step of measuring a hemoglobin level. In some embodiments, the measuring step comprises obtaining a biological sample from the subject wherein the biological sample is whole blood, serum or plasma.

In certain embodiments, the predetermined amount of the change in hemoglobin is 0.1 g/dL, 0.2 g/dL, 0.3 g/dL, 0.4 g/dL, 0.5 g/dL, 0.6 g/dL, 0.7 g/dL, 0.8 g/dL, 0.9 g/dL, 1.0 g/dL, 1.1 g/dL, 1.2 g/dL, 1.3 g/dL, 1.4 g/dL, 1.5 g/dL, 1.6 g/dL, 1.7 g/dL, 1.8 g/dL, 1.9 g/dL, 2.0 g/dL, 2.1 g/dL, 2.2 g/dL, 2.3 g/dL, 2.4 g/dL, 2.5 g/dL, 2.6 g/dL, 2.7 g/dL, 2.8 g/dL, 2.9 g/dL, 3.0 g/dL, 3.1 g/dL, 3.2 g/dL, 3.3 g/dL, 3.4 g/dL, 3.5 g/dL, 3.6 g/dL, 3.7 g/dL, 3.8 g/dL, 3.9 g/dL, 4.0 g/dL, 4.1 g/dL, 4.2 g/dL, 4.3 g/dL, 4.4 g/dL, 4.5 g/dL, 4.6 g/dL, 4.7 g/dL, 4.8 g/dL, 4.9 g/dL, 5.0 g/dL, 5.1 g/dL, 5.2 g/dL, 5.3 g/dL, 5.4 g/dL, 5.5 g/dL, 5.6 g/dL, 5.7 g/dL, 5.8 g/dL, 5.9 g/dL, 6.0 g/dL, 6.1 g/dL, 6.2 g/dL, 6.3 g/dL, 6.4 g/dL, 6.5 g/dL, 6.6 g/dL, 6.7 g/dL, 6.8 g/dL, 6.9 g/dL, 7.0 g/dL, 7.1 g/dL, 7.2 g/dL, 7.3 g/dL, 7.4 g/dL, 7.5 g/dL, 7.6 g/dL, 7.7 g/dL, 7.8 g/dL, 7.9 g/dL, 8.0 g/dL, 8.1 g/dL, 8.2 g/dL, 8.3 g/dL, 8.4 g/dL, 8.5 g/dL, 8.6 g/dL, 8.7 g/dL, 8.8 g/dL, 8.9 g/dL, 9.0 g/dL, 9.1 g/dL, 9.2 g/dL, 9.3 g/dL, 9.4 g/dL, 9.5 g/dL, 9.6 g/dL, 9.7 g/dL, 9.8 g/dL, 9.9 g/dL, 10.0 g/dL, or higher.

In certain embodiments, the predetermined maintenance dose threshold of hemoglobin is 1 g/dL, 1.5 g/dL, 2 g/dL, 2.5 g/dL, 3 g/dL, 3.5 g/dL, 4 g/dL, 4.5 g/dL, 5 g/dL, 5.5 g/dL, 6 g/dL, 6.5 g/dL, 7 g/dL, 7.5 g/dL, 8 g/dL, 8.5 g/dL, 9 g/dL, 9.5 g/dL, 10 g/dL, 10.5 g/dL, 11 g/dL, 11.5 g/dL, 12 g/dL, 12.5 g/dL, 13 g/dL, 13.5 g/dL, 14 g/dL, 14.5 g/dL, 15 g/dL, 15.5 g/dL, 16 g/dL, 16.5 g/dL, 17 g/dL, 17.5 g/dL, 18 g/dL, 18.5 g/dL, 19 g/dL, 19.5 g/dL, 20 g/dL, 20.5 g/dL or higher.

In certain embodiments, the hemoglobin level is below a predetermined loading dose threshold, and the method includes a step of administering a subsequent maintenance dose and repeating steps a-b. In certain embodiments, a hemoglobin level is at or above the predetermined maintenance dose threshold and the method includes a step of administering a reduced maintenance dose wherein the dose is reduced by a predetermined amount compared to the amount maintenance dose administered in previous step c. In some embodiments, the predetermined amount is 1%, 2%, 3%, 5%, 7%, 9%, 10%, 13%, 15%, 17%, 20%, 23%, 25%, 27%, 30%, 35%, 45%, 50%, 55%, 60%, 70%, 75%, 80%, 85%, or 95%. In alternative embodiments, the predetermined amount is 5 mg, 10 mg, 15 mg, or 20 mg.

In certain embodiments, the maintenance dosage regime comprises administering a maintenance dosage daily, twice weekly, weekly, or every 2 weeks. In certain embodiments, the maintenance dose is from 0.1 mg to 3000 mg, 1 mg to 1000 mg, 2 mg to 500 mg, 1 mg to 2000 mg, 1 mg to 1000 mg, 1 mg to 300 mg, 1 mg to 100 mg, 1 mg to 90 mg, 1 mg to 80 mg, 1 mg to 70 mg, 1 mg to 60 mg, 20 mg to 50 mg, 1 mg to 40 mg, 1 mg to 30 mg, 1 mg to 20 mg, 1 mg to 10 mg, 1 mg to 3 mg, 3 mg to 2000 mg, 3 mg to 1000 mg, 3 mg to 300 mg, 3 mg to 100 mg, 3 mg to 90 mg, 3 mg to 80 mg, 3 mg to 70 mg, 3 mg to 60 mg, 20 mg to 50 mg, 3 mg to 40 mg, 3 mg to 30 mg, 3 mg to 10 mg, 10 mg to 2000 mg, 10 mg to 1000 mg, 10 mg to 300 mg, 10 mg to 150 mg, 10 mg to 100 mg, 10 mg to 90 mg, 10 mg to 80 mg, 10 mg to 70 mg, 10 mg to 60 mg, 10 mg to 50 mg, 10 mg to 40 mg, 10 mg to 30 mg, 10 mg to 20 mg, 20 mg to 2000 mg, 20 mg to 1000 mg, 20 mg to 200 mg, 20 mg to 100 mg, 20 mg to 90 mg, 20 mg to 85 mg, 20 mg to 80 mg, 20 mg to 75 mg, 20 mg to 70 mg, 20 mg to 65 mg, 20 mg to 60 mg, 20 mg to 55 mg, 20 mg to 50 mg, 20 mg to 45 mg, 20 mg to 40 mg, 20 mg to 35 mg, 20 mg to 30 mg, 20 mg to 25 mg, 30 mg to 2000 mg, 30 mg to 1000 mg, 30 mg to 300 mg, 30 mg to 100 mg, 30 mg to 95 mg, 30 mg to 90 mg, 30 mg to 95 mg, 30 mg to 80 mg, 30 mg to 75 mg, 30 mg to 70 mg, 30 mg to 65 mg, 30 mg to 60 mg, 30 mg to 55 mg, 30 mg to 50 mg, 30 mg to 45 mg, 30 mg to 40 mg, 30 mg to 35 mg, 35 mg to 2000 mg, 35 mg to 1000 mg, 35 mg to 400 mg, 35 mg to 100 mg, 35 mg to 90 mg, 35 mg to 85 mg, 35 mg to 80 mg, 35 mg to 75 mg, 35 mg to 70 mg, 35 mg to 65 mg, 35 mg to 60 mg, 35 mg to 55 mg, 35 mg to 50 mg, 35 mg to 45 mg, 35 mg to 40 mg, 40 mg to 2000 mg, 40 mg to 1000 mg, 40 mg to 400 mg, 40 mg to 100 mg, 40 mg to 90 mg, 40 mg to 85 mg, 40 mg to 80 mg, 40 mg to 75 mg, 40 mg to 70 mg, 40 mg to 65 mg, 40 mg to 60 mg, 40 mg to 55 mg, 40 mg to 50 mg, 40 mg to 45 mg, 45 mg to 2000 mg, 45 mg to 1000 mg, 45 mg to 450 mg, 45 mg to 100 mg, 45 mg to 90 mg, 45 mg to 85 mg, 45 mg to 80 mg, 45 mg to 75 mg, 45 mg to 70 mg, 45 mg to 65 mg, 45 mg to 60 mg, 45 mg to 55 mg, 45 mg to 50 mg, 50 mg to 2000 mg, 50 mg to 1000 mg, 50 mg to 500 mg, 50 mg to 100 mg, 50 mg to 90 mg, 50 mg to 85 mg, 50 mg to 80 mg, 50 mg to 75 mg, 50 mg to 70 mg, 50 mg to 65 mg, 50 mg to 60 mg, 50 mg to 55 mg, 55 mg to 2000 mg, 55 mg to 1000 mg, 55 mg to 550 mg, 55 mg to 100 mg, 55 mg to 90 mg, 55 mg to 85 mg, 55 mg to 80 mg, 55 mg to 75 mg, 55 mg to 70 mg, 55 mg to 65 mg, 55 mg to 60 mg, 60 mg to 2000 mg, 60 mg to 1000 mg, 60 mg to 600 mg, 60 mg to 100 mg, 60 mg to 90 mg, 60 mg to 85 mg, 60 mg to 80 mg, 60 mg to 75 mg, 60 mg to 70 mg, 60 mg to 65 mg, 65 mg to 2000 mg, 65 mg to 1000 mg, 65 mg to 300 mg, 65 mg to 100 mg, 65 mg to 90 mg, 65 mg to 85 mg, 65 mg to 80 mg, 65 mg to 75 mg, 65 mg to 70 mg, 70 mg to 2000 mg, 70 mg to 1000 mg, 70 mg to 300 mg, 70 mg to 100 mg, 70 mg to 90 mg, 70 mg to 85 mg, 70 mg to 80 mg, 70 mg to 75 mg, 75 mg to 2000 mg, 75 mg to 1000 mg, 75 mg to 300 mg, 75 mg to 100 mg, 75 mg to 90 mg, 75 mg to 85 mg, 75 mg to 80 mg, 80 mg to 2000 mg, 80 mg to 1000 mg, 80 mg to 800 mg, 80 mg to 100 mg, 80 mg to 90 mg, 80 mg to 85 mg, 85 mg to 2000 mg, 85 mg to 1000 mg, 85 mg to 300 mg, 85 mg to 100 mg, 85 mg to 90 mg, 90 mg to 2000 mg, 90 mg to 1000 mg, 90 mg to 300 mg, 90 mg to 100 mg, 90 mg to 95 mg, 95 mg to 2000 mg, 95 mg to 1000 mg, 95 mg to 300 mg, 95 mg to 100 mg, 100 mg to 2000 mg, 100 mg to 1000 mg, 100 mg to 300 mg, 300 mg to 2000 mg, 300 mg to 1000 mg or 1000 mg to 2000 mg.

In certain embodiments of the methods according to the present disclosure, the maintenance dosage regime comprises administering a maintenance dosage. In certain embodiments, the maintenance dose is from 10 mg to 300 mg, 10 mg to 150 mg, 10 mg to 100 mg, 10 mg to 90 mg, 10 mg to 80 mg, 10 mg to 70 mg, 10 mg to 60 mg, 10 mg to 50 mg, 10 mg to 40 mg, 10 mg to 30 mg, 10 mg to 20 mg, 20 mg to 300 mg, 20 mg to 200 mg, 20 mg to 100 mg, 20 mg to 90 mg, 20 mg to 85 mg, 20 mg to 80 mg, 20 mg to 75 mg, 20 mg to 70 mg, 20 mg to 65 mg, 20 mg to 60 mg, 20 mg to 55 mg, 20 mg to 50 mg, 20 mg to 45 mg, 20 mg to 40 mg, 20 mg to 35 mg, 20 mg to 30 mg, 20 mg to 25 mg, 30 mg to 300 mg, 30 mg to 100 mg, 30 mg to 95 mg, 30 mg to 90 mg, 30 mg to 95 mg, 30 mg to 80 mg, 30 mg to 75 mg, 30 mg to 70 mg, 30 mg to 65 mg, 30 mg to 60 mg, 30 mg to 55 mg, 30 mg to 50 mg, 30 mg to 45 mg, 30 mg to 40 mg, 30 mg to 35 mg, 35 mg to 300 mg, 35 mg to 100 mg, 35 mg to 90 mg, 35 mg to 85 mg, 35 mg to 80 mg, 35 mg to 75 mg, 35 mg to 70 mg, 35 mg to 65 mg, 35 mg to 60 mg, 35 mg to 55 mg, 35 mg to 50 mg, 35 mg to 45 mg, 35 mg to 40 mg, 40 mg to 300 mg, 40 mg to 100 mg, 40 mg to 90 mg, 40 mg to 85 mg, 40 mg to 80 mg, 40 mg to 75 mg, 40 mg to 70 mg, 40 mg to 65 mg, 40 mg to 60 mg, 40 mg to 55 mg, 40 mg to 50 mg, 40 mg to 45 mg, 45 mg to 300 mg, 45 mg to 100 mg, 45 mg to 90 mg, 45 mg to 85 mg, 45 mg to 80 mg, 45 mg to 75 mg, 45 mg to 70 mg, 45 mg to 65 mg, 45 mg to 60 mg, 45 mg to 55 mg, 45 mg to 50 mg, 50 mg to 300 mg, 50 mg to 100 mg, 50 mg to 90 mg, 50 mg to 85 mg, 50 mg to 80 mg, 50 mg to 75 mg, 50 mg to 70 mg, 50 mg to 65 mg, 50 mg to 60 mg, 50 mg to 55 mg, 55 mg to 300 mg, 55 mg to 100 mg, 55 mg to 90 mg, 55 mg to 85 mg, 55 mg to 80 mg, 55 mg to 75 mg, 55 mg to 70 mg, 55 mg to 65 mg, 55 mg to 60 mg, 60 mg to 300 mg, 60 mg to 100 mg, 60 mg to 90 mg, 60 mg to 85 mg, 60 mg to 80 mg, 60 mg to 75 mg, 60 mg to 70 mg, 60 mg to 65 mg, 65 mg to 300 mg, 65 mg to 100 mg, 65 mg to 90 mg, 65 mg to 85 mg, 65 mg to 80 mg, 65 mg to 75 mg, 65 mg to 70 mg, 70 mg to 300 mg, 70 mg to 100 mg, 70 mg to 90 mg, 70 mg to 85 mg, 70 mg to 80 mg, 70 mg to 75 mg, 75 mg to 300 mg, 75 mg to 100 mg, 75 mg to 90 mg, 75 mg to 85 mg, 75 mg to 80 mg, 80 mg to 300 mg, 80 mg to 100 mg, 80 mg to 90 mg, 80 mg to 85 mg, 85 mg to 300 mg, 85 mg to 100 mg, 85 mg to 90 mg, 90 mg to 300 mg, 90 mg to 100 mg, 90 mg to 95 mg, 95 mg to 300 mg, 95 mg to 100 mg, 100 mg to 300 mg, 100 mg to 150 mg, or 150 mg to 300 mg.

In additional embodiments, the methods according to the present disclosure include a dosage reduction regime comprising the steps of:
c) administering the maintenance dose; and
d) determining if a biomarker level is above, at or below a predetermined maintenance dose threshold or determining if a change in biomarker level is above, at, or below a predetermined amount, wherein:
i) if the biomarker level is below the predetermined maintenance dose threshold or if the change in biomarker level is below the predetermined amount, then administering a subsequent maintenance dose and repeating steps c-d; or
ii) if the biomarker level is at or above the predetermined maintenance dose threshold or if the change in biomarker level is at or above the predetermined amount, then administering a reduced maintenance dose wherein the dosage is reduced by a predetermined amount, and optionally repeating steps c-d.

In additional embodiments, the methods according to the present disclosure include a dosage reduction regime comprising the steps of:
c) administering a maintenance dose; and
d) determining if a biomarker level is above, at, or below a predetermined maintenance dose threshold, wherein:
   i) if the biomarker level is below a predetermined maintenance dose threshold, then administering a subsequent maintenance dose and repeating steps c-d; or
   ii) if the biomarker level is at or above the predetermined maintenance dose threshold, then administering a reduced maintenance dose wherein the dosage is reduced by a predetermined amount, and optionally repeating steps d-e.

In certain embodiments, the biomarker is one or more selected from hepcidin; iron metabolism markers including iron, ferritin, transferrin, soluble transferrin receptor [STR], and total iron binding capacity [TIBC]; cytokines including CRP, EPO, IL-6, and TGF-beta 1; and indicators of inhibition of signal transduction pathways including phosphorylation of SMAD-1, 2, 3, 5 and 8 in PBMCs.

In certain embodiments, determining step d) further comprises a step of measuring a biomarker level. In some embodiments, the measuring step comprises obtaining a biological sample from the subject wherein the biological sample is whole blood, serum or plasma or a bone marrow aspirate.

In certain embodiments, the biomarker level is below a predetermined loading dose threshold, and the method includes a step of administering a subsequent maintenance dose and repeating steps a-b. In certain embodiments, a subsequent biomarker level is at or above the predetermined maintenance dose threshold and the method includes a step of administering a reduced maintenance dose wherein the dosage is reduced by a predetermined amount compared to the maintenance dose administered in previous step c. In some embodiments, the predetermined amount is 1%, 2%, 3%, 5%, 7%, 9%, 10%, 13%, 15%, 17%, 20%, 23%, 25%, 27%, 30%, 35%, 45%, 50%, 55%, 60%, 70%, 75%, 80%, 85%, or 95%.

In additional embodiments, the methods according to the present disclosure include a method of determining the efficacy of the methods of treating an ALK5-mediated disorder disclosed herein, comprising the steps of:
a) determining a baseline level of hemoglobin in said subject;
b) determining a change in a level of hemoglobin from baseline after said administration step;
wherein if the hemoglobin level has increased from baseline by a predetermined amount, the method of administering the compound of structure (I) for treatment is determined to be efficacious.

In certain embodiments, the hemoglobin level has increased by 1.5 g/dL from baseline.

In additional embodiments, the methods according to the present disclosure include a method of determining the efficacy of treatment comprising the steps of:
a) determining a baseline level of hemoglobin in said subject;
b) determining a subsequent level of hemoglobin after said administration step;
wherein if the hemoglobin level is 10 g/dL or more, the method of administering the compound of structure (I) for treatment is determined to be efficacious.

In additional embodiments, the methods according to the present disclosure include a method of determining the efficacy of treatment comprising the steps of:
a) determining a baseline amount of a biomarker in said subject;
b) determining a change in a biomarker level from baseline after said administration step;
wherein if the biomarker has increased or decreased from baseline by a predetermined amount, the method of administering the compound of structure (I) for treatment is determined to be efficacious.

In further embodiments, the biomarker is selected from hepcidin in serum and bone marrow aspirate; iron metabolism markers in serum selected from iron, ferritin, transferrin, soluble transferrin receptor [STR], and total iron binding capacity [TIBC]; cytokines in serum or plasma selected from CRP, EPO, IL-6, and TGF-beta 1; and indicators of inhibition of signal transduction pathways in bone marrow aspirates selected from phosphorylation of SMAD-1, 2, 3, 5 and 8 in PBMCs.

In particular embodiments of the methods of determining the efficacy of treatment, the biomarker is hepcidin obtained from a blood plasma of said subject.

In certain instances, it may be advantageous to administer the compound of structure (I), or a pharmaceutically acceptable salt or prodrug thereof, in combination with one or more therapeutically active agents independently selected from anti-cancer agents, anti-allergic agents, anti-emetics, pain relievers, immunomodulators and cytoprotective agents.

The term “combination therapy” refers to the administration of two or more therapeutic agents to treat a therapeutic disease, disorder or condition described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. The compound of structure (I), or a pharmaceutically acceptable salt or prodrug thereof, and additional therapeutic agents can be administered via the same administration route or via different administration routes. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the diseases, conditions or disorders described herein.

General Chemotherapeutic agents considered for use in combination therapies include capecitabine (Xeloda®), N4-pentoxycarbonyl-5-deoxy-5-fluorocytidine, carboplatin (Paraplatin®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), doxorubicin hydrochloride (Adriamycin®, Rubex®), fludarabine phosphate (Fludara®), 5-fluorouracil (Adrucil®, Efudex®), Gemcitabine (difluorodeoxycitidine), irinotecan (Camptosar®), L-asparaginase (ELSPAR®), 6-mercaptopurine (Purinethol®), methotrexate (Folex®), pentostatin, 6-thioguanine, thiotepa, and topotecan hydrochloride for injection (Hycamptin®).

Anti-cancer agents of particular interest for combinations with a compound of structure (I), or a pharmaceutically acceptable salt or prodrug thereof, include:

Purine antimetabolites and/or inhibitors of de novo purine synthesis: pemetrexed (Alimta®), gemcitabine (Gemzar®), 5-fluorouracil (Adrucil®, Carac® and Efudex®), methotrexate (Trexall®), capecitabine (Xeloda®), floxuridine (FUDR®), decitabine (Dacogen®), azacitidine (Vidaza® and Azadine®), 6-mercaptopurine (Purinethol®), cladribine (Leustatin®, Litak® and Movectro®), fludarabine (Fludara®), pentostatin (Nipent®), nelarabine (Arranon®), clofarabine (Clolar® and Evoltra®), and cytarabine (Cytosar®).

MTAP inhibitors: (3R,4S)-1-((4-amino-5H-pyrrolo[3,2-d]pyrimidin-7-yl)methyl)-4-((methylthio)methyl)pyrrolidin-3-ol (MT-DADMe-Immucillin-A, CAS 653592-04-2).

Methylthioadenosine: ((2R,3R,4S,5S)-2-(6-amino-9H-purin-9-yl)-5-((methylthio)methyl)tetrahydrofuran-3,4-diol, CAS 2457-80-9).

Epidermal growth factor receptor (EGFR) inhibitors: Erlotinib hydrochloride (Tarceva®) and Gefitnib (Iressa®).

EGFR antibodies: Cetuximab (Erbitux®).

MET inhibitors: Capmatinib (INC280, CAS 1029712-80-8).

Platelet-derived Growth Factor (PDGF) receptor inhibitors: Imatinib (Gleevec®); Linifanib (N-[4-(3-amino-1H-indazol-4-yl)phenyl]-N′-(2-fluoro-5-methylphenyl)urea, also known as ABT 869, available from Genentech); Sunitinib malate (Sutent®); Quizartinib (AC220, CAS 950769-58-1); Pazopanib (Votrient®); Axitinib (Inlyta®); Sorafenib (Nexavar®); Vargatef (BIBF1120, CAS 928326-83-4); Telatinib (BAY57-9352, CAS 332012-40-5); Vatalanib dihydrochloride (PTK787, CAS 212141-51-0); and Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470).

Phosphoinositide 3-kinase (PI3K) inhibitors: 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730); 4-(trifluoromethyl)-5-(2,6-dimorpholinopyrimidin-4-yl)pyridin-2-amine (also known as BKM120 or NVP-BKM120, and described in PCT Publication No. WO2007/084786); Alpelisib (BYL719): (5Z)-5-[[4-(4-Pyridinyl)-6-quinolinyl]methylene]-2,4-thiazolidinedione (GSK1059615, CAS 958852-01-2); 5-[8-methyl-9-(1-methylethyl)-2-(4-morpholinyl)-9H-purin-6-yl]-2-pyrimidinamine (VS-5584, CAS 1246560-33-7) and everolimus)(AFINITOR®.

Cyclin-Dependent Kinase (CDK) inhibitors: Ribociclib (LEE011, CAS 1211441-98-3); Aloisine A; Alvocidib (also known as flavopiridol or HMR-1275, 2-(2-chlorophenyl)-5,7-dihydroxy-8-[(3 S,4R)-3-hydroxy-1-methyl-4-piperidinyl]-4-chromenone, and described in U.S. Pat. No. 5,621,002); Crizotinib (PF-02341066, CAS 877399-52-5); 2-(2-Chlorophenyl)-5,7-dihydroxy-8-[(2R,3S)-2-(hydroxymethyl)-1-methyl-3-pyrrolidinyl]-4H-1-benzopyran-4-one, hydrochloride (P276-00, CAS 920113-03-7); 1-Methyl-5-[[2-[5-(trifluoromethyl)-1H-imidazol-2-yl]-4-pyridinyl]oxy]-N-[4-(trifluoromethyl)phenyl]-1H-benzimidazol-2-amine (RAF265, CAS 927880-90-8); Indisulam (E7070); Roscovitine (CYC202); 6-Acetyl-8-cyclopentyl-5-methyl-2-(5-piperazin-1-yl-pyridin-2-ylamino)-8H-pyrido[2,3-d]pyrimidin-7-one, hydrochloride (PD0332991); Dinaciclib (SCH727965); N-[5-[[(5-tert-Butyloxazol-2-yl)methyl]thio]thiazol-2-yl]piperidine-4-carboxamide (BMS 387032, CAS 345627-80-7); 4-[[9-Chloro-7-(2,6-difluorophenyl)-5H-pyrimido[5,4-d][2]benzazepin-2-yl]amino]-benzoic acid (MLN8054, CAS 869363-13-3); 5-[3-(4,6-Difluoro-1H-benzimidazol-2-yl)-1H-indazol-5-yl]-N-ethyl-4-methyl-3-pyridinemethanamine (AG-024322, CAS 837364-57-5); 4-(2,6-Dichlorobenzoylamino)-1H-pyrazole-3-carboxylic acid N-(piperidin-4-yl)amide (AT7519, CAS 844442-38-2); 4-[2-Methyl-1-(1-methylethyl)-1H-imidazol-5-yl]-N-[4-(methylsulfonyl)phenyl]-2-pyrimidinamine (AZD5438, CAS 602306-29-6); Palbociclib (PD-0332991); and (2R,3R)-3-[[2-[[3-[[S(R)]—S-cyclopropylsulfonimidoyl]-phenyl]amino]-5-(trifluoromethyl)-4-pyrimidinyl]oxy]-2-butanol (BAY 10000394). In embodiments, the CDK inhibitor is a CDK9 inhibitor. In embodiments, the CDK inhibitor is alvocidib or a prodrug thereof. In embodiments, the CDK inhibitor is a prodrug of alvocidib. Such prodrugs are described in International Application No. PCT/US2016/033099, which is incorporated by reference in its entirety for its teachings regarding the same. In embodiments, the CDK inhibitor is a phosphate prodrug of alvocidib. In certain embodiments, the phosphate prodrug of alvocidib has the following structure (II):

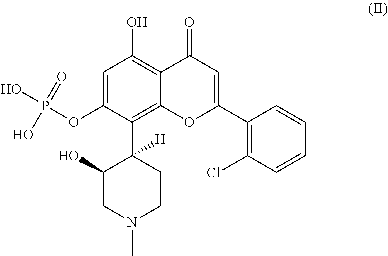 [SMILES: CN1CC[C@H](c2c(OP(=O)(O)O)cc(O)c3c(=O)cc(-c4ccccc4Cl)oc23)[C@H](O)C1]

p53-MDM2 inhibitors: (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, [(4S,5R)-2-(4-tert-butyl-2-ethoxyphenyl)-4,5-bis(4-chlorophenyl)-4,5-dimethylimidazol-1-yl]-[4-(3-methylsulfonylpropyl)piperazin-1-yl]methanone (RG7112), 4-[[(2R,3S,4R,5S)-3-(3-chloro-2-fluorophenyl)-4-(4-chloro-2-fluorophenyl)-4-cyano-5-(2,2-dimethylpropyl)pyrrolidine-2-carbonyl]amino]-3-methoxybenzoic acid (RG7388), SAR299155, 2-((3R,5R,6S)-5-(3-chlorophenyl)-6-(4-chlorophenyl)-1-((S)-1-(isopropylsulfonyl)-3-methylbutan-2-yl)-3-methyl-2-oxopiperidin-3-yl)acetic acid (AMG232), {(3R,5R,6S)-5-(3-Chlorophenyl)-6-(4-chlorophenyl)-1-[(2S,3S)-2-hydroxy-3-pentanyl]-3-methyl-2-oxo-3-piperidinyl}acetic acid (AM-8553), (±)-4-[4,5-Bis(4-chlorophenyl)-2-(2-isopropoxy-4-methoxy-phenyl)-4,5-dihydro-imidazole-1-carbonyl]-piperazin-2-one (Nutlin-3), 2-Methyl-7-[Phenyl(phenylamino)methyl]-8-quinolinol (NSC 66811), 1-N-[2-(1H-indol-3-yl)ethyl]-4-N-pyridin-4-ylbenzene-1,4-diamine (JNJ-26854165), 4-[4,5-bis(3,4-chlorophenyl)-2-(2-isopropoxy-4-methoxy-phenyl)-4,5-dihydro-imidazole-1-carboxyl]-piperazin-2-one (Caylin-1), 4-[4,5-bis(4-trifluoromethyl-phenyl)-2-(2-isopropoxy-4-methoxy-phenyl)-4,5-dihydro-imidazole-1-carboxyl]-piperazin-2-one (Caylin-2), 5-[[3-Dimethylamino)propyl]amino]-3,10-dimethylpyrimido[4,5-b]quinoline-2,4(3H,10H)-dione dihydrochloride (HLI373) and trans-4-Iodo-4′-boranyl-chalcone (SC204072).

Mitogen-activated protein kinase (MEK) inhibitors: XL-518 (also known as GDC-0973, Cas No. 1029872-29-4, available from ACC Corp.); Selumetinib (5-[(4-bromo-2-chlorophenyl)amino]-4-fluoro-N-(2-hydroxyethoxy)-1-methyl-1H-benzimidazole-6-carboxamide, also known as AZD6244 or ARRY 142886, described in PCT Publication No. WO2003077914); 2-[(2-Chloro-4-iodophenyl)amino]-N-(cyclopropylmethoxy)-3,4-difluoro-benzamide (also known as CI-1040 or PD184352 and described in PCT Publication No. WO2000035436); N-[(2R)-2,3-Dihydroxypropoxy]-3,4-difluoro-2-[(2-fluoro-4-iodophenyl)amino]-benzamide (also known as PD0325901 and described in PCT Publication No. WO2002006213); 2,3-Bis[amino[(2-aminophenyl)thio]methylene]-butanedinitrile (also known as U0126 and described in U.S. Pat. No. 2,779,780); N-[3,4-Difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO2007014011); (3S,4R,5Z,8S,9S,11E)-14-(Ethylamino)-8,9,16-trihydroxy-3,4-dimethyl-3,4,9, 19-tetrahydro-1H-2-benzoxacyclotetradecine-1,7(8H)-dione] (also known as E6201 and described in PCT Publication No. WO2003076424); 2′-Amino-3′-methoxyflavone (also known as PD98059 available from Biaffin GmbH & Co., KG, Germany); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); Pimasertib (AS-703026, CAS 1204531-26-9); Trametinib dimethyl sulfoxide (GSK-1120212, CAS 1204531-25-80); 2-(2-Fluoro-4-iodophenylamino)-N-(2-hydroxyethoxy)-1,5-dimethyl-6-oxo-1,6-dihydropyridine-3-carboxamide (AZD 8330); 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655); 0; and 5-[(4-Bromo-2-fluorophenyl)amino]-4-fluoro-N-(2-hydroxyethoxy)-1-methyl-1H-Benzimidazole-6-carboxamide (MEK162).

B-RAF inhibitors: Regorafenib (BAY73-4506, CAS 755037-03-7); Tuvizanib (AV951, CAS 475108-18-0); Vemurafenib (Zelboraf®, PLX-4032, CAS 918504-65-1); Encorafenib (also known as LGX818); 1-Methyl-5-[[2-[5-(trifluoromethyl)-1H-imidazol-2-yl]-4-pyridinyl]oxy]-N-[4-(trifluoromethyl)phenyl-1H-benzimidazol-2-amine (RAF265, CAS 927880-90-8); 5-[1-(2-Hydroxyethyl)-3-(pyridin-4-yl)-1H-pyrazol-4-yl]-2,3-dihydroinden-1-one oxime (GDC-0879, CAS 905281-76-7); 5-[2-[4-[2-(Dimethylamino)ethoxy]phenyl]-5-(4-pyridinyl)-1H-imidazol-4-yl]-2,3-dihydro-1H-Inden-1-one oxime (GSK2118436 or SB590885); (+/−)-Methyl (5-(2-(5-chloro-2-methylphenyl)-1-hydroxy-3-oxo-2,3-dihydro-1H-isoindol-1-yl)-1H-benzimidazol-2-yl)carbamate (also known as XL-281 and BMS908662), dabrafenib (Tafinlar®), and N-(3-(5-chloro-1H-pyrrolo[2,3-b]pyridine-3-carbonyl)-2,4-difluorophenyl)propane-1-sulfonamide (also known as PLX4720).

ALK inhibitors: Crizotinib (Xalkori®).

Some subjects may experience allergic reactions to a compound of structure (I), or a pharmaceutically acceptable salt or prodrug thereof, and/or other anti-cancer agent(s) during or after administration; therefore, anti-allergic agents are often administered to minimize the risk of an allergic reaction. Suitable anti-allergic agents include corticosteroids (Knutson, S., et al., PLoS One, DOI: 10.1371/journal.pone.0111840 (2014)), such as dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®).

Some subjects may experience nausea during and after administration of the compound of structure (I), or a pharmaceutically acceptable salt or prodrug thereof, and/or other anti-cancer agent(s); therefore, anti-emetics are used in preventing nausea (upper stomach) and vomiting. Suitable anti-emetics include aprepitant (Emend®), ondansetron (Zofran®), granisetron HCl (Kytril®), lorazepam (Ativan®. dexamethasone (Decadron®), prochlorperazine (Compazine®), casopitant (Rezonic® and Zunrisa®), and combinations thereof.

Medication to alleviate the pain experienced during the treatment period is often prescribed to make the subject more comfortable. Common over-the-counter analgesics, such Tylenol®, are often used. However, opioid analgesic drugs such as hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), and fentanyl (e.g., Duragesic®) are also useful for moderate or severe pain.

Immunomodulators of particular interest for combinations with a compound of structure (I), or a pharmaceutically acceptable salt or prodrug thereof, include: Afutuzumab (available from Roche®); Pegfilgrastim (Neulasta®); Lenalidomide (CC-5013, Revlimid®); Thalidomide (Thalomid®), Actimid (CC4047); and IRX-2 (mixture of human cytokines including interleukin 1, interleukin 2, and interferon γ, CAS 951209-71-5, available from IRX Therapeutics).

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. Suitable cytoprotective agents include Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid).

The structure of the active compounds identified by code numbers, generic or trade names may be taken from the actual edition of the standard compendium “The Merck Index” or from databases, e.g. Patents International (e.g. IMS World Publications).

In one embodiment, the present disclosure provides pharmaceutical compositions comprising a compound of structure (I), or a pharmaceutically acceptable salt or prodrug thereof, together with a pharmaceutically acceptable carrier suitable for administration to a subject, either alone or together with other anti-cancer agents.

In particular, compositions will either be formulated together as a combination therapeutic or administered separately.

In one embodiment, the present disclosure provides a pharmaceutical combination comprising an effective amount of a compound of structure (I), or a pharmaceutically acceptable salt, or prodrug thereof, and one or more therapeutically active agents. In embodiments, a pharmaceutical combination comprises a pharmaceutically acceptable salt of a compound of structure (I). In some embodiments, a pharmaceutical combination comprises a pharmaceutically acceptable acid addition salt of a compound of structure (I). In particular embodiments, a pharmaceutical combination comprises a hydrochloric acid salt of a compound of structure (I).

In combination therapy for treatment of MDS, a compound of structure (I), or a pharmaceutically acceptable salt or prodrug thereof, and other anti-cancer agent(s) may be administered simultaneously, concurrently or sequentially with no specific time limits, wherein such administration provides therapeutically effective levels of the two compounds in the body of the subject.

In an embodiment, the compound of structure (I), or a pharmaceutically acceptable salt or prodrug thereof, and the other anti-cancer agent(s) is generally administered sequentially in any order by infusion or orally. The dosing regimen may vary depending upon the stage of the disease, physical fitness of the subject, safety profiles of the individual drugs, and tolerance of the individual drugs, as well as other criteria well-known to the attending physician and medical practitioner(s) administering the combination. The compound of structure (I), or a pharmaceutically acceptable salt or prodrug thereof, and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another aspect of the present disclosure, a kit comprising two or more separate pharmaceutical compositions, at least one of which contains a compound of structure (I), or a pharmaceutically acceptable salt or prodrug thereof, is provided. In one embodiment, the kit comprises means for separately retaining said compositions, such as a container, divided bottle, or divided foil packet. An example of such a kit is a blister pack, as typically used for the packaging of tablets, capsules and the like.

The kit of the present disclosure may be used for administering different dosage forms, for example, oral and parenteral, for administering the separate compositions at different dosage intervals, or for titrating the separate compositions against one another. To assist compliance, the kit of the present disclosure typically comprises directions for administration.

A compound of structure (I), or a pharmaceutically acceptable salt or prodrug thereof, may also be used to advantage in combination with known therapeutic processes, for example, the administration of hormones or especially radiation.

In the combination therapies of the present disclosure, the compound of structure (I), or a pharmaceutically acceptable salt or prodrug thereof, and the other therapeutic agent may be manufactured and/or formulated by the same or different manufacturers. Moreover, the compound of structure (I), or a pharmaceutically acceptable salt or prodrug thereof, and the other therapeutic (or pharmaceutical agent) may be brought together into a combination therapy: (i) prior to release of the combination product to physicians (e.g. in the case of a kit comprising the compound of structure (I), or a pharmaceutically acceptable salt or prodrug thereof, and the other therapeutic agent); (ii) by the physician themselves (or under the guidance of the physician) shortly before administration; (iii) in the subject themselves, e.g. during sequential administration of the compound of structure (I), or a pharmaceutically acceptable salt or prodrug thereof, and the other therapeutic agent.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition or combination of the present disclosure can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound of structure (I), or a pharmaceutically acceptable salt or prodrug thereof, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties may be demonstrable in vitro and in vivo tests using advantageously mammals, e.g., mice, rats, dogs, monkeys or isolated organs, tissues and preparations thereof. A compound of structure (I), or a pharmaceutically acceptable salt or prodrug thereof, can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

### Pharmacology and Utility

MDS is a collection of hematological conditions (e.g., refractory anemia, refractory anemia with ringed sideroblasts, refractory anemia with excess blasts, refractory anemia with excess blasts in transformation, refractory cytopenia with multilineage dysplasia, and myelodysplastic syndrome associated with an isolated 5q chromosome abnormality) characterized by ineffective production of myeloid blood cells. In MDS subjects, blood stem cells do not mature into healthy red blood cells, white blood cells, or platelets. Accordingly, most MDS subjects are afflicted with chronic anemia. Therefore, MDS subjects eventually require blood transfusions and/or treatment with growth factors (e.g., erythropoietin or G-CSF) to increase red blood cell levels. However, the frequency of such therapies can have tissue and organ damage from the buildup of extra iron.

It has been shown that the TGF-β pathway is overactive in MDS. For example, SMAD2 is activated in bone marrow precursor cells and is overexpressed in gene expression profiles of MDS cells. Inhibition of some members of this pathway (e.g., ALK5) has been shown to promote hematopoiesis in MDS.

Therefore, ALK5 represents an attractive target for the development of a novel therapy for the treatment of MDS. In particular, the need exists for small molecules that inhibit the activity of ALK5. It has now been found that a compound of structure (I), or a pharmaceutically acceptable salt or prodrug thereof, is useful to treat ALK5-mediated diseases or disorders, for example, MDS. In one embodiment, the compound of structure (I), or a pharmaceutically acceptable salt or prodrug thereof, is useful to treat MDS.

Provided herein are methods for treating MDS comprising administering an effective amount of a compound of structure (I), or a pharmaceutically acceptable salt or prodrug thereof, to a subject in need thereof.

Also provided herein are methods for treating anemia and methods of treating anemia of chronic disease (ACD), said methods comprising administering an effective amount of a compound of structure (I), or a pharmaceutically acceptable salt or prodrug thereof, to a subject in need thereof. In certain embodiments, the subject has or is identified as being at risk of having MDS.

Also provided herein are methods for reducing transfusion frequency in a subject in need thereof, the method comprising administering an effective amount of a compound of structure (I) or a pharmaceutically acceptable salt, or prodrug thereof, to the subject.

Also provided herein are methods for reducing transfusion dependence in a subject in need thereof, the method comprising administering an effective amount of a compound of structure (I) or a pharmaceutically acceptable salt, or prodrug thereof, to the subject.

In particular embodiments of the methods provided herein, the compound of structure (I) is a crystalline salt which may be an acid addition salt such as a hydrochloric acid salt, a monovalent hydrochloric acid salt, an anhydrous acid addition salt, or a salt of Form A as provided herein.

In certain embodiments of the methods provided herein, the subject has anemia associated with MDS.

In certain embodiments of the methods provided herein, the subject has anemia of chronic disease associated with MDS.

In certain embodiments of the methods provided herein, the subject has transfusion dependent anemia associated with MDS.

In certain embodiments of the methods provided herein, the subject has MDS with single lineage dysplasia refractory anemia.

In certain embodiments of the methods provided herein, the subject has MDS with ring sideroblasts and is intolerant, resistant or refractory to luspatercept.

In embodiments of any one of the methods of the present disclosure, the method comprises improving one or more hematologic parameters in a subject, wherein the hematologic parameter is selected from decreasing myoblasts, increasing hemoglobin, increasing platelets, increasing neutrophils, decreasing hepcidin, reducing units of red blood cell transfused, reducing frequency of transfusion, and reducing transfusion dependence.

In certain embodiments of the methods described herein, an effective amount of the compound of structure (I) improves one or more hematologic parameters in a subject, wherein the hematologic parameter is selected from decreasing myoblasts, increasing hemoglobin, increasing platelets, increasing neutrophils, decreasing hepcidin, reducing units of red blood cell transfused, reducing frequency of transfusion, and reducing transfusion dependence.

In certain embodiments of the methods described herein, decreasing myoblasts is characterized as wherein myoblasts are decreased i) to be 5% or fewer of bone marrow cells; or ii) by 50% or more compared to a baseline amount measured prior to administration of the compound of structure (I). In certain embodiments, the decrease in myoblasts is maintained for 4 weeks, 8 weeks, or 12 weeks consecutively, after administration of the compound of structure (I).

In certain embodiments of the methods described herein, increasing hemoglobin is defined as increasing hemoglobin to 10 g/dL or more. For example, 10.5 g/dL, 11 g/dL, 11.5 g/dL, 12 g/dL, 12.5 g/dL, 13 g/dL, 13.5 g/dL, 14 g/dL or more.

In certain embodiments, increasing hemoglobin is defined as increasing hemoglobin by 1.5 g/dL or more compared to an amount measured prior to administration of the compound of structure (I). For example, by 2 g/dL, 2.5 g/dL, 3 g/dL, 3.5 g/dL, 4 g/dL, 4.5 g/dL, or more.

In certain embodiments of the methods described herein, the increase in hemoglobin occurs in the absence of red blood cell transfusions.

In certain embodiments of the methods described herein, the increase in hemoglobin is maintained for 4 weeks, 8 weeks, or 12 weeks in the absence of red blood cell transfusions.

In certain embodiments of the methods described herein, increasing platelets is characterized as increasing the platelet count by 1×10⁹/L, 3×10⁹/L, 5×10⁹/L, 8×10⁹/L, 10×10⁹/L, 15×10⁹/L, 20×10⁹/L, 25×10⁹/L, 30×10⁹/L, 35×10⁹/L, 40×10⁹/L, 45×10⁹/L, 50×10⁹/L, 55×10⁹/L, 60×10⁹/L or more. In certain embodiments, this increase is an increase over baseline amount measured before administration of the compound of structure (I).

In certain embodiments of the methods described herein, increasing platelets is characterized as increasing the platelet count to 55×10⁹/L, 60×10⁹/L, 65×10⁹/L, 70×10⁹/L, 75×10⁹/L, 80×10⁹/L, 85×10⁹/L, 90×10⁹/L, 95×10⁹/L, 100×10⁹/L, 110×10⁹/L, 120×10⁹/L, 130×10⁹/L, 140×10⁹/L, 150×10⁹/L, 160×10⁹/L or more. In certain embodiments, the increase in platelets is for subjects having a baseline amount of 50×10⁹/L or more.

In certain embodiments of the methods described herein, the increase in platelets of any of the embodiments described above is maintained for 4 weeks, 8 weeks, or 12 weeks in the absence of red blood cell transfusions.

In certain embodiments of the methods described herein, increasing neutrophils is characterized as increasing the neutrophil count by 0.1×10⁹/L, 0.15×10⁹/L, 0.2×10⁹/L, 0.25×10⁹/L, 0.3×10⁹/L, 0.35×10⁹/L, 0.4×10⁹/L, 0.45×10⁹/L, 0.5×10⁹/L, 0.55×10⁹/L, 0.6×10⁹/L, 0.65×10⁹/L, 0.7×10⁹/L, 0.75×10⁹/L, 0.8×10⁹/L, 0.85×10⁹/L, 0.9×10⁹/L, 1.0×10⁹/L or more. In certain embodiments, this increase is an increase over baseline amount measured before administration of the compound of structure (I).

In certain embodiments of the methods described herein, increasing platelets is characterized as increasing the neutrophil count 0.6×10⁹/L, 0.65×10⁹/L, 0.7×10⁹/L, 0.75×10⁹/L, 0.8×10⁹/L, 0.85×10⁹/L, 0.9×10⁹/L, 0.95×10⁹/L, 1.0×10⁹/L, 1.05×10⁹/L, 1.1×10⁹/L, 1.15×10⁹/L, 1.2×10⁹/L, 1.25×10⁹/L, 1.3×10⁹/L, 1.35×10⁹/L, 1.4×10⁹/L, 1.45×10⁹/L, 1.5×10⁹/L, 1.55×10⁹/L, 1.6×10⁹/L, 1.65×10⁹/L, 1.7×10⁹/L, 1.75×10⁹/L, 1.8×10⁹/L, 1.85×10⁹/L, 1.9×10⁹/L, 1.95×10⁹/L, 2.0×10⁹/L or more. In certain embodiments, the increase in neutrophils is for subjects having a baseline amount of 0.5×10⁹/L or more.

In certain embodiments of the methods described herein, the increase in neutrophils of any of the embodiments described above is maintained for 4 weeks, 8 weeks, or 12 weeks in the absence of red blood cell transfusions.

In certain embodiments of the methods described herein, decreasing hepcidin is characterized as decreasing hepcidin by 10%, 15%, 20%, 25%, 30%, 35%, 40%, 45%, 50%, 55%, 60%, 65%, 70%, 75%, 80%, 85%, 90%, 95%, or more compared to baseline amount measured prior to administration of the compound of structure (I).

In certain embodiments of the methods described herein, the method comprises reducing the units of red blood cell transfused, wherein units of red blood cells transfused is reduced i) by 4 or more units; or ii) by 50% or more; for a period of time after administration of the compound of structure (I) compared to the units of red blood cells transfused for the same period of time prior to administration of the compound of structure (I). In certain embodiments, the period of time is 4 weeks, 8 weeks, or 12 weeks.

In some embodiments, “reducing transfusion frequency” is characterized by (1) a reduction in the number of transfusions prescribed by a competent medical professional over a specified interval (e.g., 4 weeks, 4 weeks, 1 month, 3 months, 6 months, etc.) after administration of a compound of structure (I), or a pharmaceutically acceptable salt or prodrug thereof, as compared to the number of transfusions prescribed in the same amount of time prior to administration; and/or (2) a reduction in the number of transfusions received over a specified interval after administration of a compound of structure (I), or a pharmaceutically acceptable salt or prodrug thereof, as compared to the number of transfusions received in the same amount of time prior to administration.

In certain embodiments, “transfusion dependence” encompasses a condition of severe anemia that requires that a subject receive ≥1 blood transfusions over a specified interval (e.g., 1 month, 3 months, 6 months, etc.). A reduction in transfusion dependence is characterized by to (1) an increase in the specified interval in which a subject requires ≥1 blood transfusions; or (2) elimination of the subject's need to receive blood transfusions.

Another embodiment provides a method for treating a subject having or at risk of developing MDS, the method comprising administering to the subject a composition comprising an effective amount of a compound of structure (I), or a pharmaceutically acceptable salt or prodrug thereof.

In some embodiments, the methods described herein involve identifying a subject being at risk of developing MDS. In some embodiments, the methods described herein further include administering an effective amount of a compound of structure (I), or a pharmaceutically acceptable salt or prodrug thereof, to a subject identified as being at risk of developing MDS. In some embodiments, the methods further include administering an effective amount of a compound of structure (I), or a pharmaceutically acceptable salt or prodrug thereof, to a subject suspected to have MDS.

In some embodiments, provided are methods for prophylactically treating MDS comprising administering an effective amount of a compound of structure (I), or a pharmaceutically acceptable salt or prodrug thereof, to a subject in need thereof. In some embodiments, provided are methods for prophylactically treating MDS comprising administering an effective amount of a compound of structure (I), or a pharmaceutically acceptable salt or prodrug thereof, to a subject in need thereof.

In some embodiments, provided are methods for preventing MDS comprising administering an effective amount of a compound of structure (I), or a pharmaceutically acceptable salt or prodrug thereof, to a subject in need thereof. In some embodiments, provided are methods for preventing MDS comprising administering an effective amount of a compound of structure (I), or a pharmaceutically acceptable salt or prodrug thereof, to a subject in need thereof.

In another aspect, a method is provided for treating a subject having or at risk of developing MDS, the method comprising administering to the subject in need thereof an effective amount of a compound of structure (I), or a pharmaceutically acceptable salt or prodrug thereof.

In embodiments, treating MDS comprises reducing transfusion frequency in the subject, reducing transfusion dependence in the subject, or both. “Reducing transfusion frequency” refers to (1) a reduction in the number of transfusions prescribed by a competent medical professional over a specified interval (e.g., 1 month, 3 months, 6 months, etc.) after administration of a compound of structure (I), or a pharmaceutically acceptable salt or prodrug thereof, as compared to the number of transfusions prescribed in the same amount of time prior to administration; and/or (2) a reduction in the number of transfusions received over a specified interval after administration of a compound of structure (I), or a pharmaceutically acceptable salt or prodrug thereof, as compared to the number of transfusions received in the same amount of time prior to administration. “Transfusion dependence” refers to a condition of severe anemia that requires that a subject receive ≥1 blood transfusions over a specified interval (e.g., 1 month, 3 months, 6 months, etc.). A reduction in transfusion dependence refers to (1) an increase in the specified interval in which a subject requires ≥1 blood transfusions; or (2) elimination of the subject's need to receive blood transfusions.

Provided herein are methods for treating MDS comprising administering an effective amount of a compound of structure (I), or a pharmaceutically acceptable salt or prodrug thereof, to a subject in need thereof, wherein the method comprises reducing transfusion frequency. Some embodiments provide a method for reducing transfusion frequency comprising administering an effective amount of a compound of structure (I), or a pharmaceutically acceptable salt or prodrug thereof, to a subject in need thereof.

Another embodiment provides a method for treating a subject having or at risk of developing MDS, the method comprising administering to the subject a composition comprising an effective amount of a compound of structure (I), or a pharmaceutically acceptable salt or prodrug thereof, wherein the method comprises reducing transfusion frequency.

In some embodiments, the methods described herein involve identifying a subject being at risk of developing MDS. In some embodiments, the methods described herein further include administering an effective amount of a compound of structure (I), or a pharmaceutically acceptable salt or prodrug thereof, to a subject identified as being at risk of developing MDS, wherein the administering reduces transfusion frequency. In some embodiments, the methods further include administering an effective amount of a compound of structure (I), or a pharmaceutically acceptable salt or prodrug thereof, to a subject suspected to have MDS, wherein the administering reduces transfusion frequency.

In some embodiments, provided are methods for prophylactically treating MDS comprising administering an effective amount of a compound of structure (I), or a pharmaceutically acceptable salt or prodrug thereof, to a subject in need thereof, wherein the method comprises reducing transfusion frequency. In some embodiments, provided are methods for prophylactically reducing transfusion frequency, comprising administering an effective amount of a compound of structure (I), or a pharmaceutically acceptable salt or prodrug thereof, to a subject in need thereof.

In some embodiments, provided are methods for preventing MDS comprising administering an effective amount of a compound of structure (I), or a pharmaceutically acceptable salt or prodrug thereof, to a subject in need thereof, wherein the preventing comprises reducing transfusion frequency. In some embodiments, provided are methods for reducing transfusion frequency comprising administering an effective amount of a compound of structure (I), or a pharmaceutically acceptable salt or prodrug thereof, to a subject in need thereof.

In another aspect, a method is provided for treating a subject having or at risk of developing MDS, the method comprising administering to the subject in need thereof an effective amount of a compound of structure (I), or a pharmaceutically acceptable salt or prodrug thereof, wherein the method comprises reducing transfusion frequency.

Provided herein are methods for treating MDS comprising administering an effective amount of a compound of structure (I), or a pharmaceutically acceptable salt or prodrug thereof, to a subject in need thereof, wherein the method comprises reducing transfusion dependence. Some embodiments provide a method for reducing transfusion dependence comprising administering an effective amount of a compound of structure (I), or a pharmaceutically acceptable salt or prodrug thereof, to a subject in need thereof.

Another embodiment provides a method for treating a subject having or at risk of developing MDS, the method comprising administering to the subject a composition comprising an effective amount of a compound of structure (I), or a pharmaceutically acceptable salt or prodrug thereof, wherein the method comprises reducing transfusion dependence.

In some embodiments, the methods described herein involve identifying a subject being at risk of developing MDS. In some embodiments, the methods described herein further include administering an effective amount of a compound of structure (I), or a pharmaceutically acceptable salt or prodrug thereof, to a subject identified as being at risk of developing MDS, wherein the administering reduces transfusion dependence. In some embodiments, the methods further include administering an effective amount of a compound of structure (I), or a pharmaceutically acceptable salt or prodrug thereof, to a subject suspected to have MDS, wherein the administering reduces transfusion dependence.

In some embodiments, provided are methods for prophylactically treating MDS comprising administering an effective amount of a compound of structure (I), or a pharmaceutically acceptable salt or prodrug thereof, to a subject in need thereof, wherein the method comprises reducing transfusion dependence. In some embodiments, provided are methods for prophylactically reducing transfusion dependence, comprising administering an effective amount of a compound of structure (I), or a pharmaceutically acceptable salt or prodrug thereof, to a subject in need thereof.

In some embodiments, provided are methods for preventing MDS comprising administering an effective amount of a compound of structure (I), or a pharmaceutically acceptable salt or prodrug thereof, to a subject in need thereof, wherein the preventing comprises reducing transfusion dependence. In some embodiments, provided are methods for reducing transfusion dependence comprising administering an effective amount of a compound of structure (I), or a pharmaceutically acceptable salt or prodrug thereof, to a subject in need thereof.

In another aspect, a method is provided for treating a subject having or at risk of developing MDS, the method comprising administering to the subject in need thereof an effective amount of a compound of structure (I), or a pharmaceutically acceptable salt or prodrug thereof, wherein the method comprises reducing transfusion dependence.

In embodiments, methods of the disclosure comprise administering an effective amount of a pharmaceutically acceptable salt of a compound of structure (I). In some embodiments, methods of the disclosure comprise administering an effective amount of a pharmaceutically acceptable acid addition salt of a compound of structure (I). In particular embodiments, methods of the disclosure comprise administering an effective amount of a hydrochloric acid salt of a compound of structure (I).

In some embodiments of the methods disclosed herein, the subject has primary MDS. In other embodiments of the methods disclosed herein, the subject has secondary MDS.

As is understood, MDS can also be classified as very low risk, low-risk, intermediate risk or high-risk as determined by the guidance published by Greenberg, Tuechler, Schanz et al., Revised International Prognostic Scoring System (IPSS-R) for Myelodysplastic Syndrome, Blood 120: 2454, 2012, and set forth herein.

IPSS-R Cytogenetic Risk Groups

| Cytogenetic | |
|---|---|
| prognostic subgroups | Cytogenetic abnormalities |
| Very good | −Y, del(11q) |
| Good | Normal, del(5q), del(12p), del(20q), double including |
| | del(5q) |
| Intermediate | del(7q), +8, +19, i(17q), any other single or double |
| | independent clones |
| Poor | −7, inv(3)/t(3q)/del(3q), double including −7/del(7q), |
| | Complex: 3 abnormalities |
| Very poor | Complex: >3 abnormalities |

See, Schanz J. et al., J. Clin. Oncology 2012; 30:820) and Greenberg, Tuechler, Schanz et al, Revised International Prognostic Scoring System (IPSS-R) for Myelodysplastic Syndrome, Blood 120: 2454, 2012.

IPSS-R Prognostic Score Values

| Prognostic | | | | | | | |
|---|---|---|---|---|---|---|---|
| variable | 0 | 0.5 | 1 | 1.5 | 2 | 3 | 4 |
| Cytogenetics | Very | | Good | | Intermediate | Poor | Very |
| | Good | | | | | | Poor |
| BM Blast % | <=2 | | >2-<5% | | 5-10% | >10% | |
| Hemoglobin | =>10 | | 8-<10 | <8 | | | |
| Platelets | =>100 | 50-<100 | <50 | | | | |
| ANC | =>0.8 | <0.8 | | | | | |

### See, Greenberg, Tuechler, Schanz et al, Revised International Prognostic Scoring System (IPSS-R) for Myelodysplastic Syndrome, Blood 120: 2454, 2012.

IPSS-R Prognostic Risk Categories/Scores

| RISK CATEGORY | RISK SCORE |
|---|---|
| Very Low | <=1.5 |
| Low | >1.5-3 |
| Intermediate | >3-4.5 |
| High | >4.5-6 |
| Very High | >6 |

### See, Greenberg, Tuechler, Schanz et al, Revised International Prognostic Scoring System (IPSS-R) for Myelodysplastic Syndrome, Blood 120: 2454, 2012.

In some embodiments of the methods disclosed herein, the subject has high-risk MDS, i.e., an IPSS-R risk score of >4.5-6. In other embodiments of the methods disclosed herein, the subject has low-risk MDS, i.e., an IPSS-R risk score of >1.5-3. In other embodiments of the methods disclosed herein, the subject has very low-risk MDS, i.e., an IPSS-R risk score of <=1.5. In other embodiments of the methods disclosed herein, the subject has intermediate-risk MDS, i.e., an IPSS-R risk score of >3-4.5.

In various embodiments, a subject has received previous treatment for MDS. In such embodiments, a subject may be refractory to or intolerant of the previous treatment, such as erythropoiesis-stimulating agents (ESAs), including recombinant human erythropoietin and darbepoietin. In certain embodiments, the subject is refractory or resistant to prior ESA treatment, as defined by any one of the following: Refractory to prior ESA treatment—documentation of non-response or response that is no longer maintained to prior ESA-containing regimen, either as single agent or combination (e.g., with G-CSF) wherein the ESA regimen must have been either: recombinant human erythropoietin (rHu EPO) >40,000 IU/wk for at least 8 doses or equivalent; or darbepoetin alpha >500 i.tg Q3W for at least 4 doses or equivalent. In another embodiment, the subject is intolerant to prior ESA treatment—documentation of discontinuation of prior ESA containing regimen, either as single agent or combination (e.g., with G-CSF), at any time after introduction due to intolerance or an adverse event.

In alternative embodiments, the subject is ESA treatment naïve or ESA treatment ineligible. In certain embodiments, the subject has baseline endogenous serum erythropoietin level EPO plasma levels of greater than 200 IU.

In certain embodiments, the subject has confirmed lower risk MDS (IPSS Low/INT-1 or IPSS-R Very Low, Low, Intermediate-1). In some embodiments the MDS is de novo (primary). In some embodiments, the MDS is secondary.

In certain embodiments, subjects with 5q deletions have failed or are intolerant of lenalidomide (sold under the trade name Revlimid® among others) treatment.

In certain embodiments, subjects were previously treated for anemia with or without RBC transfusion support. In some embodiments, the subject is “transfusion-free” (Tf) with anemia (hemoglobin less than 10 g/dL, without transfusions). In some embodiments, the subject has “Low transfusion burden” (LTb), defined as requiring less than 4 red blood cell units in the 8 weeks before treatment and optionally a baseline hemoglobin <10 g/dL. In some embodiments, the subject is transfusion dependent and has a “High transfusion burden” (HTb), defined as requiring 4 or more red blood cell units in the 8 weeks before treatment.

In particular embodiments, all previous therapy with ESAs, G-CSF and GM-CSF is discontinued 14 days or more before treatment by any of the methods provided by the present disclosure.

Other embodiments provide a method for selecting a treatment regimen and for treating a disease in a subject based on the subject having a predetermined genetic profile. In various embodiments, methods of the disclosure further comprise obtaining a sample from a subject and determining a genetic profile.

Embodiments provided herein include methods for selecting a treatment regimen for a subject based on the subject's genetic profile. Such genetic profiles may be produced in any suitable manner (e.g., microarrays, reverse transcription polymerase chain reaction (RT-PCR), RNA/DNA sequencing, etc.).

In some embodiments, the genetic profile comprises one or more mutations in a gene selected from ASXL1, BCOR, BRAF, CALR, CBL, CEBPA, CSF3R, DDX41, DNMT3A, ETNK1, ETV6, EZH2, GATA2, GNAS, GNB1, IDH1, IDH2, JAK2, KIT, KRAS, MPL, NF1, NPM1, NRAS, PDGFRA, PHF6, PPM1D, PTPN11, RAD21, RUNX1, SETBP1, SF3B1, SH2B3, SMC1A, SMC3, SRSF2, STAG2, STAT3, STAT5B, TET2, TP53, U2AF1, WT1 and ZRSR2. In further embodiments, the genetic profile comprises one or more mutations in a gene selected from ACVR1, AVCR1B, ACVR2A, ACVR2B, ACVRL1, BMPR1A, TGFBR1, BMPR1B, TGFB1, TGFB2, TGFB3, IL6R, BMP6, SMAD1, SMAD2, SMAD3, SMAD5, SMAD8 (SMAD9), and HAMP. The term “gene” can include not only coding sequences but also regulatory regions such as promoters, enhancers, and termination regions. The term further can include all introns and other DNA sequences spliced from the mRNA transcript, along with variants resulting from alternative splice sites. Gene sequences encoding the particular protein can be DNA or RNA that directs the expression of the particular protein. These nucleic acid sequences may be a DNA strand sequence that is transcribed into RNA or an RNA sequence that is translated into the particular protein. The nucleic acid sequences include both the full-length nucleic acid sequences as well as non-full-length sequences derived from the full-length protein.

In various embodiments, the subject receiving treatment has one or more mutations in a gene selected from ASXL1, BCOR, BRAF, CALR, CBL, CEBPA, CSF3R, DDX41, DNMT3A, ETNK1, ETV6, EZH2, GATA2, GNAS, GNB1, IDH1, IDH2, JAK2, KIT, KRAS, MPL, NF1, NPM1, NRAS, PDGFRA, PHF6, PPM1D, PTPN11, RAD21, RUNX1, SETBP1, SF3B1, SH2B3, SMC1A, SMC3, SRSF2, STAG2, STAT3, STAT5B, TET2, TP53, U2AF1, WT1 and ZRSR2. In further embodiments, the genetic profile comprises one or more mutations in a gene selected from ACVR1, AVCR1B, ACVR2A, ACVR2B, ACVRL1, BMPR1A, TGFBR1, BMPR1B, TGFB1, TGFB2, TGFB3, IL6R, BMP6, SMAD1, SMAD2, SMAD3, SMAD5, SMAD8 (SMAD9), and HAMP. gene. In embodiments, the subject has a predetermined genetic profile comprising such mutation(s). In some embodiments, the one or more mutations in the gene comprise a missense mutation, a frameshift mutation, a duplication (i.e. copy number variation), a splice site mutation, or a combination thereof.

A compound of structure (I), or a pharmaceutically acceptable salt or prodrug thereof, exhibit valuable pharmacological properties, which can be demonstrated at least by using any one of the following test procedures.

Also included are methods of inhibiting ALK5, the method comprising administering a compound of structure (I). In certain embodiments is provided a method for inhibiting ALK5 activity in a subject, the method comprising administering an effective amount of a compound of structure (I): or a pharmaceutically acceptable salt, or prodrug thereof, to the subject. In particular embodiments of the methods for inhibiting ALK5 provided herein, the compound of structure (I) is a crystalline salt which may be an acid addition salt such as a hydrochloric acid salt, a monovalent hydrochloric acid salt, an anhydrous acid addition salt, or a salt of Form A as provided herein.

In certain embodiments, the method comprising contacting cells expressing ALK5 with an effective amount of a compound of structure (I). In certain embodiments, the cells are in vitro.

Also included are methods of inhibiting ALK5 activity in a cell, the method comprising administering to the cell a compound of structure (I) in an amount effective to inhibit ALK5. In certain embodiments, the cell is in vitro.

In certain embodiments of the above methods, inhibition is measured by pSMAD 2/3 phosphorylation. In further embodiments, the measured IC50 is 200 nM, 220 nM, 240 nM, 260 nM, 280 nM, 300 nM, 320 nM or higher. In particular embodiments, the measured IC₅₀ is 280 nM or higher.

In certain embodiments of the above methods, inhibition is measured by nanobret assay. In further embodiments, the measured IC₅₀ is 1.5 μM, 1.6 μM, 1.7 μM, 1.8 μM, 1.9 μM, 2.1 μM, 2.2 μM, 2.3 μM or higher. In further embodiments, the measured IC₅₀ is 2.0 μM or higher.

In certain embodiments of the above methods, inhibition is measured by SMAD reporter (RDSR) assay. In further embodiments, the measured IC₅₀ is 60 nM, 70 nM, 80 nM, 90 nM, 100 nM, 110 nM, 120 nM, 130 nM, 140 nM, 150 nM, 160 nM, 170 nM, 180 nM, 190 nM, 200 nM, 220 nM, 240 nM, 250 nM, 260 nM, 280 nM, 300 nM, 320 nM or higher. In further embodiments, the measured IC₅₀ is 2.0 μM or more.

### Example 1

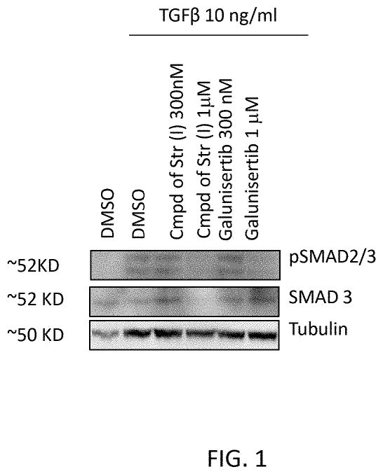

An HCl salt of the compound of structure (I) was assessed for its effect on SMAD 2/3 phosphorylation in biochemical assays and cellular assays. The effect of compounds of the present disclosure on TGFβ induced SMAD 2/3 phosphorylation was assessed in Panc-1 pancreatic cells, and the results are shown in FIG. 1.

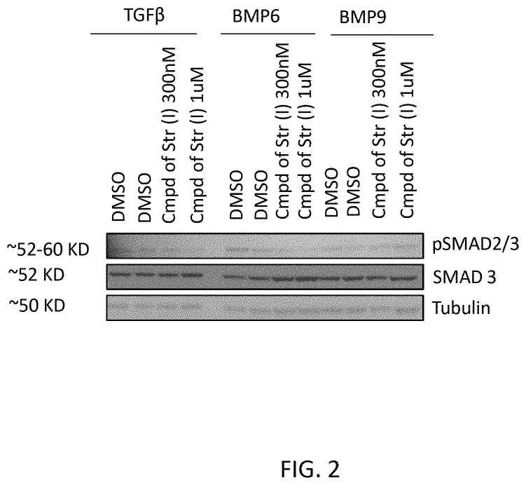

The effect of compound of structure (I) on TGF (3, BMP 6, BMP9 induced SMAD 2/3 phosphorylation in MOLM-13 AML cells, and the results are shown in FIG. 2. Cells were pre-treated with the compound of structure (I) for two hours, then treated with stimulant for 30 minutes before lysis.

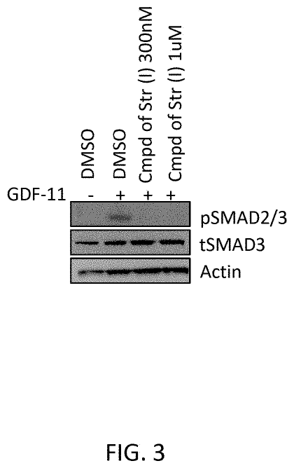

The effect of a compound of structure (I) was on growth differentiation factor 11 (GDF 11) induced SMAD 2/3 phosphorylation in K562 chronic myelogenous leukemia (CIVIL) cells, and the results are shown in FIG. 3. Cells were pre-treated with the compound of structure (I) for two hours, then treated with stimulant for 30 minutes before lysis.

### Example 2

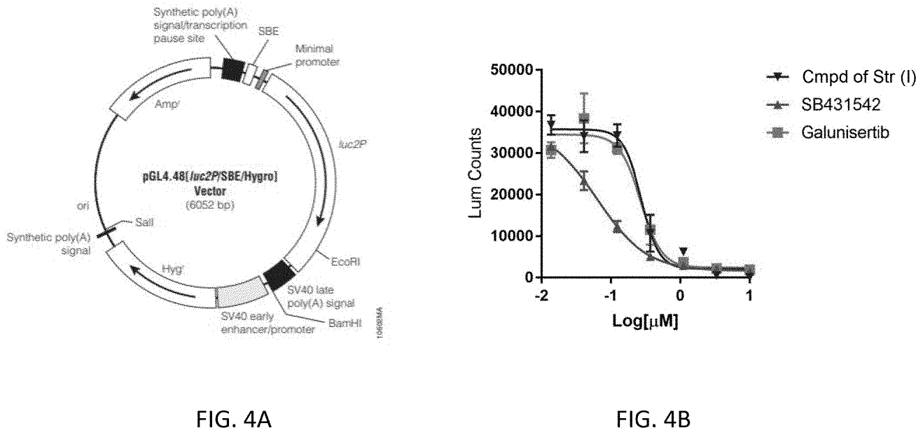

An HCl salt of the compound of structure (I) was tested in a Rhabdomyosarcoma (RD) cell SMAD reporter (RDSR) assay. The RD cell line was transfected with the pGL4.48(luc2P/SBE/Hygro) vector (from Promega, see, FIG. 4A) and cultured in the presence of hygromycin (200 μg/mL at start, 100 μg/mL to maintain) for several weeks, or until established. For testing of the compound of structure (I), transfected cells were pretreated with drug, and then induced with 50 pg/mL TGFβ1 for up to 24 hours.

In this reporter assay, luminescence was developed via the expression of a luciferase, which is controlled by a SMAD binding element (SBE). In optimization experiments, strong and specific dependence on ALK5 for development of a signal was seen. In this assay, the compound of structure (I) achieved an IC₅₀ of 309 nM. The known ALK5 inhibitors, SB431542 and galunisertib, showed IC₅₀s of 84.7 and 299 nM, respectively. The results are shown in FIG. 4B.

### Example 3

An HCl salt of the compound of structure (I) was tested in an ALK5 nanobret assay. HEK293 cells were transfected with the ALK5-Nanoluc fusion (Promega) vector, which encodes for a luciferase tagged form of ALK5. For testing of the compound of structure (I), transfected cells were pretreated with drug. Subsequently, a fluorescent tracer was added and the fluorescence signal was measured.

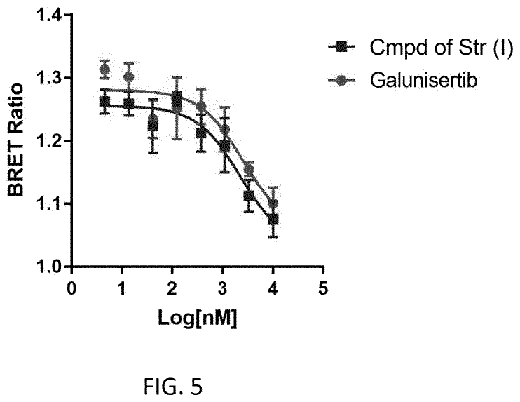

In this assay, luminescence develops via the nanoluciferase tagged ALK5, which can transfer signal via bioluminescence energy transfer (BRET) to the tracer, yielding a fluorescent signal. This allows for detection of the presence of the tracer in the active site, or its competition, via an inhibitor, and subsequent loss of signal. In this assay, the compound of structure (I) achieved an IC₅₀ of 2.3 μM. The known ALK5 inhibitor, galunisertib, achieved an IC₅₀ of 2.6 μM. The results are shown in FIG. 5.

### Example 4

Male and/or female NUP98-HOXD13 mice (e.g., three months old) are treated, e.g., twice-weekly, with an HCl salt of the compound of structure (I) or vehicle. Wildtype mice are dosed with compound of structure (I) or vehicle and served as controls. Blood samples are collected before the first dose is administered and at regular (e.g., monthly) intervals thereafter to perform CBC measurements.

### Example 5

To further examine the efficacy of an HCl salt of the compound of structure (I) in vivo, it is tested in a transgenic mouse expressing a fusion gene (Alb/TGF) consisting of modified porcine TGF-01 cDNA under the control of the regulatory elements of the mouse albumin gene. These mice constitutively secrete TGF-β, become anemic, and have histologic marrow findings that mimic human MDS, thus serving as an in vivo model of bone marrow failure.

Mice are randomized into treatment or placebo groups on the basis of pretreatment hematocrits. Mice are given compound of structure (I) by gastric lavage using a curved 14 G needle. Blood counts are measured after 14 days of administration of the compound of structure (I) or vehicle. Blood counts are analyzed by Advia machine. Mice femurs are flushed and bone marrows cells are used for clonogenic assays.

### Example 6

A Phase 1/2 Trial of Oral Compound of Structure (I) in Subjects with MDS

A Phase 1/2, open-label clinical study is performed to determine preliminary safety and efficacy of the compound of structure (I) to treat anemia when administered to adult subjects with very low, low, or intermediate-1 (IPSS-R) MDS. The recommended Phase 2 dose (recommended dose) will be determined by the maximum tolerated dose (MTD) or maximum administered dose (MAD) in the Phase 1 portion of the study.

Enrollment is as follows:

Phase 1—Single agent dose escalation: ˜30 subjects (evaluable, completing Cycle 1)
Phase 2—Expansion arms
Arm 1-20˜40 subjects
Arm 2—20˜40 subjects
Total: ˜60-110 subjects

### Phase 1—Dose Escalation

Subjects will receive a daily 20 mg dose of the compound of structure (I) starting on Cycle 1, Day 1. Dose escalation is planned to proceed with subjects receiving each dose level of −40 mg, 60 mg, 90 mg, 120 mg, 160 mg, 210 mg, 270 mg and further respective dose increments of up to 25% from 1 dose cohort to the next may continue until one of the following occurs:

Maximum tolerated dose (MTD) for the Phase 2—Dose Expansion portion of the study is determined.

Dose escalation may be stopped at the maximum administrated dose (MAD) determination based on totality of safety data and medical considerations by the safety review committee (SRC).

Dose escalation will be performed using a design based on a 2-parameter Bayesian logistic regression model (BLRM) (Neuenschwander, 2008). The BLRM method will be applied along with the escalation with overdose control (EWOC) principle to control the risk of exposing subjects to toxic doses (Babb, 1998). Based on this principle, a dose level will be considered safe if the probability of excessive toxicity, i.e., the probability of a DLT rate over 33% is no greater than 25%. MTD with estimated posterior probability of a DLT within target toxicity interval (16%, 33%) among the admissible doses fulfilling EWOC is determined by BLRM. MTD is estimated based on observed DLTs.

The use of Bayesian adaptive models for Phase 1 studies has been advocated by the European Medicines Agency's guideline on clinical trials in small populations (European Medicines Agency, 2016).

After completing a given dose cohort, the decision to adjust the dose (de-escalate the dose to dose level-1 (10 mg) or to a previous dose level or escalate the dose to the next dose level) or stay at the same dose will be made by the study SRC, based on review of adverse events, DLT's SAE's laboratory data, PK. The actual dose level to be tested in the next cohort will be chosen based on the above risk assessment, using the BLRM method. The dose recommended by the BLRM method will be treated as guidance and will be integrated with a clinical assessment of the safety adverse event information and review of clinical data, including above safety and PK data. Intermediate doses between planned dose levels may be explored based on safety consideration. The BLRM method estimates the MTD by updating the probability of observing a DLT for each dose level in the study as DLT information becomes available. Additional arm with different dose schedule may be considered based on clinical judgment supported by medical observations.

### Phase 2—Dose Expansion

Phase 2 will determine the preliminary efficacy of the compound of structure (I) in two expansion arms of up to 40 subjects each; Arm 1 will enroll subjects who are

refractory or resistant to prior ESA treatment and Arm 2 will enroll subjects who are ESA naïve or ineligible with EPO plasma levels of >200 IU.

Efficacy in case of response rate will be monitored using the Bayesian posterior probability.

Investigational product, dosage and mode of administration:

The compound of structure (I) is administered PO and should be taken in the morning after an overnight fast with up to 200 mL or 7 ounces of water at least 1 hour before ingesting any food or other medications. There is no rest period between cycles (4 weeks (28 days)).

Subjects exhibiting treatment benefit up to 24 weeks may continue up to 336 days (48 weeks) of treatment, unless treatment is terminated due to progression of disease, loss of hematological response, unacceptable toxicity, withdrawal of consent, or any other reason. Treatment beyond 48 weeks will be considered for subjects deriving clinical benefit with therapy.

Note: Loss of hematological response (lack of response or refractory to further treatment) will follow the progression/relapse after hematological improvement by IWG 2006.

### Phase 1

The compound of structure (I) dosing in the Phase 1 dose escalation period of the study will follow the daily dosing schedule based on the planned escalation levels at Table 1.

TABLE 1
Planned Dose Escalation Levels

| Dose Level | Dose (Daily Schedule) | |
|---|---|---|
| −1 | 10 | mg |
| 1 | 20 | mg |
| 2 | 40 | mg |
| 3 | 60 | mg |
| 4 | 90 | mg |
| 5 | 120 | mg |
| 6 | 160 | mg |
| 7 | 210 | mg |
| 8 | 270 | mg |

Note that an intermediate dose level between the planned dose levels are contemplated. In addition, based on safety during the escalation phase, additional dose schedules than those outlined in the table may be explored.

### Phase 2

The Phase 2 study will use the maximum administrated dose/recommended dose from the Phase 1 study. Response rate will be monitored using the Bayesian posterior probability. Efficacy will be monitored.

All responding subjects are eligible to receive the compound of structure (I) in the absence of MDS disease progression or until loss of hematological response or unacceptable toxicity.

### Assessments:

### Safety Assessments:

### Phase 1 and Phase 2

Safety and tolerability of the compound of structure (I) will be assessed by analyzing DLTs and rates of treatment-emergent adverse events (TEAEs) summarized within treatment group(s) at the MedDRA preferred term and primary system organ class levels, dose interruptions and dose reductions. Similar summaries will be made for subsets of AEs such as (1) those judged by the Investigator to be related to study treatment, and (2) serious adverse events (SAEs). Adverse events will be graded according to NCI CTCAE v5.0.

Other routine safety assessments (e.g., physical examinations, vital sign measurements, intensive ECG monitoring, echocardiography, cardiac/hepatic MRI's, history of cardiac symptoms, cardiac safety markers, serum ferritin level and clinical laboratory testing (hematology and chemistry)) will be evaluated as measures of safety and tolerability for the entire study duration. These assessments will be summarized by patient safety listing; shift tables and treatment group using mean, standard deviation, median, minimum and maximum changes from baseline values.

Collect and document complete transfusion history for a minimum of 12 weeks immediately preceding the first dose of the compound of structure (I). This transfusion data must include hemoglobin measured prior to transfusion (pre-transfusion Hgb).

### Efficacy Assessments:

### Phase 1 and Phase 2

Efficacy will be assessed by:
Hematology (e.g. hemoglobin, neutrophils and platelets)
RBC transfusions (number of units and frequency)
Bone marrow aspirate for assessment of MDS disease (e.g. morphology, cytogenetics)
AML transformation

Survival will be collected up to one year from the start of patient's treatment with the compound of structure (I).

Criteria for evaluation:

Pharmacokinetics:

PK parameters will be assessed including:
Cmax=maximum observed plasma concentration on first dose.
Ctrough=trough plasma concentrations.
Tmax=time to Cmax (peak time).
AUCτ=AUC within a dosing interval.
Additional parameters may be determined.

Plasma concentrations of the compound of structure (I) will be summarized by descriptive statistics, including mean, n, standard deviation, coefficient of variation, minimum, maximum, and median. Prior to analysis of study samples, the assay sensitivity, specificity, linearity, and reproducibility will be documented.

Phase 1—Dose Escalation:

Plasma PK analyses for the compound of structure (I) and possibly metabolites, if any, and dose proportionality will be determined.

Pharmacokinetic assessments have been scheduled on protocol visit dates when patients will be at the study site for other protocol required assessments:

PK Sampling Scheme:

Cycle:
Week 1:
   Cycle 1 Day 1: pre-dose (time zero), 0.5, 2, 4, 6, 8, 10 hours
   Cycle 1 Day 2: pre-dose (time zero, synonymous with Day 1, 24 hour)
   Cycle 1 Day 4: pre-dose (time zero), 0.5, 2, 4, 6, 8 hours
Week 2
   Cycle 1 Day 8: pre-dose (time zero), 0.5, 2, 4, 6, 8, 10 hours
Week 3
   Cycle 1. Day 15: pre-dose (time zero), 8 hours
Week 4
   Cycle 1 Day 22: pre-dose (rime zero), 8 hours

Cycle 2:
Week 5
   Cycle 2 Day 1: pre-dose (time zero), 8 hours
Week 6
   Cycle 2 Day 8: pre-dose (time zero), 8 hours
Week 7
   Cycle 2 Day 15: pre-dose (time zero), 0.5, 2, 4, 6, 8, 10 hours
   Cycle 2 Day 16: pre-dose (time zero, synonymous with Day 15, 24 hour)

| PK Sampling | Cycle 1 | | | | | | Cycle 2 | | | |
|---|---|---|---|---|---|---|---|---|---|---|
| Scheme for Phase-1 | Week 1 | | | Week 2 | Week 3 | Wk 4 | Week 5 | Week 6 | Week 7 | |
| Time (HR) | C1D1 | C1D2 | C1D4 | C1D8 | C1D15 | C1D22 | C2D1 | C2D8 | C2D15 | C2D16 |
| Pre-dose | ✓ | ✓ | ✓ | ✓ | ✓ | ✓ | ✓ | ✓ | ✓ | ✓ |
| 0.5 | ✓ | | ✓ | ✓ | | | | | ✓ | |
| 2 | ✓ | | ✓ | ✓ | | | | | ✓ | |
| 4 | ✓ | | ✓ | ✓ | | | | | ✓ | |
| 6 | ✓ | | ✓ | ✓ | | | | | ✓ | |
| 8 | ✓ | | ✓ | ✓ | ✓ | ✓ | ✓ | ✓ | ✓ | |
| 10 | ✓ | | | ✓ | | | | | ✓ | |

Each sample “✓” is 3 mL
Total number of samples = 37
Total volume over 7 weeks = 111 mL

Phase 2—Dose Expansion:

The compound of structure (I) plasma concentration data at various timepoints. Specifically, pre-dose (trough) samples on Day 1 of each of Week 4 (Cycle 1 Day 22), 5 (Cycle 2 Day 1), 6 (Cycle 2 D8), 7 (Cycle2 Day 15), and 9 (Cycle 3 Day 1).

| | PK Sampling Scheme for Phase 2 | | | | |
|---|---|---|---|---|---|
| | Week 4 | Week 5 | Week 6 | Week 7 | Week 9 |
| | (Cycle 1) | (Cycle 2) | (Cycle 2) | (Cycle 2) | (Cycle 3) |
| | C1D22 | C2D1 | C2D8 | C2D15 | C3D1 |
| Time (hr) | | | | | |
| Pre-done | ✓ | ✓ | ✓ | ✓ | ✓ |

Each sample “✓” is 3 mL
Total number of samples = 37
Total volume over 7 weeks = 111 mL

Biomarker Assessments and Endpoints:

Peripheral blood and bone marrow samples will be collected at protocol-specific time points to assess the effects of the compound of structure (I). The samples will be used to determine any possible correlation between the rate of erythropoietic efficacy response, clinically positive bone marrow aspirate results, and biomarkers for the compound of structure (I).

The types of biomarkers to be analyzed may include, but are not limited to, nucleic acids, proteins, lipids or metabolites. Biomarker assessments may be used to assess and generate prognostic, predictive, or surrogate biomarker signatures. These assessments may be explored in the context of MDS or related conditions or drugs of similar class. Analyses will include evaluating genetic mutations and other biomarkers associated with MDS.

Biomarkers include, but are not limited to:
Hepcidin in serum and bone marrow aspirate.
Iron metabolism in serum: (e.g. serum iron, ferritin, transferrin, soluble transferrin receptor [STR], and total iron binding capacity [TIBC]).
Cytokine panel, including CRP, EPO, IL-6, TGF-beta1 in serum and/or plasma.
Signal transduction pathways inhibited by the compound of structure (I), including phosphorylation of SMAD-1, 2, 3, 5 and 8, in PBMCs and bone marrow aspirates.
Gene mutations associated with MDS and/or associated with signal transduction pathways inhibited by the compound of structure (I) in bone marrow aspirates and/or peripheral blood samples.
Bone effect biomarkers—Bone specific alkaline phosphatase (BSALP), C-terminal and N-terminal Type 1 Collagen Telopeptide (CTX/NTX) in serum.

Additional analyses is performed based on the following data collected:
Hematology assessment (e.g., red blood cell [RBC] count, complete blood count [CBC], white blood cell [WBC] with differential, hemoglobin, hematocrit, nucleated red blood cells [nRBC], absolute reticulocyte count, platelet count, mean corpuscular volume [MCV], mean corpuscular hemoglobin [MCH], mean corpuscular hemoglobin concentrations [MCHC], and red blood cell distribution width [RDW]), weekly from Cycle Day 1 to Cycle 3 Day 1. Then starting at Cycle 3, every Cycle Day 1 and Day 15, EOT and at post-treatment visit.
Full serum chemistry panel to include: blood urea nitrogen, phosphorus, magnesium, lactate dehydrogenase, creatinine, uric acid, total protein, albumin, calcium, glucose, total bilirubin, direct bilirubin, alkaline phosphatase, aspartate aminotransferase, alanine aminotransferase, and electrolytes (sodium, potassium, chloride, CO2). Cycle 1—weekly, Cycle 2 biweekly, staring at Cycle 3, every Cycle Day 1, EOT and at post-treatment visit.
Coagulation panel [PT & aPTT] and fibrinogen from Cycle 3 onward Day 1 of every Cycle and EOT.
Iron panel (serum iron, ferritin, transferrin, soluble transferrin [STR], and

TIBC), including hepcidin—pre-dose, Cycle 1—weekly, Cycle 2 and 3 biweekly (D1 and D15), Cycle 4+D1—every 4 weeks through Cycle 7 D1 (Week 24). Note: Ferritin will be used as a safety parameter.

Phase 2—Changes from Baseline BFI

The Brief Fatigue Inventory (BFI) is a brief participant-reported questionnaire that measures the severity of fatigue based on the worst fatigue experienced during the past 24-hours. The severity of fatigue is assessed using an 11-point numeric scale, with 0=no fatigue and 10=fatigue as bad.

### Inclusion and Exclusion Criteria:

### Phase 1 and Phase 2

1. Subjects with confirmed lower risk MDS (IPSS Low/INT-1 or IPSS-R Very Low, Low, Intermediate-1), de novo or secondary.
2. Subjects with 5q deletions are allowed only if they have failed or are intolerant of lenalidomide treatment.
3. Subjects previously treated with anemia with or without RBC transfusion support:
a) Transfusion-free (Tf) with anemia (hemoglobin <10 g/dL, without transfusions).
b) Low transfusion burden (LTb), defined as requiring less than 4 red blood cell units in the 8 weeks before treatment (and baseline hemoglobin <10 g/dL).
c) High transfusion burden (HTb), defined as requiring 4 or more red blood cell units in the 8 weeks before treatment (transfusion-dependent).
4. Written, signed consent for trial participation must be obtained from the patient appropriately in accordance with applicable ICH guidelines and local and regulatory requirements prior to the performance of any study specific procedure.
5. Must be ≥18 years of age.
6. Subjects with an Eastern Cooperative Oncology Group (ECOG) Performance Status (PS) score ≤2.
7. Subjects with a life expectancy of ≥3 months (90 days) per the treating investigator.
8. Subjects with adequate major organ functions meeting the following criteria on the basis of laboratory data within 4 weeks (28 days) before enrollment (if multiple data are available, most recent data during the period):
Serum creatinine: ≤1.8× the upper limit of the normal (ULN) range.
Total bilirubin ≤1.5× upper limit of normal (ULN) except in subjects with Gilbert's syndrome. Subjects with Gilbert's syndrome may enroll if direct bilirubin ≤2.0×ULN of the direct bilirubin. Elevated indirect bilirubin due to post-transfusion hemolysis is allowed.
Aspartate transaminase (AST) and alanine transaminase (ALT): ≤2.5×ULN.
Left ventricular ejection fraction (LVEF) ≥45% by echocardiogram or multigated acquisition (MUGA) scan.
   9. All previous therapy with ESAs, G-CSF and GM-CSF must be discontinued ≥14 days before treatment.
   10. Twenty-eight day (4 week) washout period from prior treatment with HMAs (hypomethylating agents), ImiDs (immunomodulatory imide drugs), luspatercept and/or investigational drugs.
   11. Women of child bearing potential (WOCBP) must have a negative serum or urine pregnancy test within 5 days prior to the first dose of the compound of structure (I).
   12. Non-fertile or agree to use an adequate method of contraception while on study and for 7 months following the study and have a negative pregnancy test (if female of childbearing potential) and not currently nursing; males agree to use an adequate method of contraception while on study and for 4 month following the study.
   13. Subjects must be able to comply with the requirements of the entire study and accessible for treatment and follow-up.
   14. Patient agrees not to participate in other interventional clinical studies during their participation in this trial, while on study treatment. Subjects participating in surveys or observational studies are eligible to participate in this study.
   Phase 2 (Inclusion criteria below are only for Phase 2 participants)

### Arm 1

Refractory or resistant to prior ESA treatment, as defined by any one of the following:

Refractory to prior ESA treatment—documentation of non-response or response that is no longer maintained to prior ESA-containing regimen, either as single agent or combination (e.g., with G-CSF); ESA regimen must have been either:
Recombinant human erythropoietin (rHu EPO) ≥40,000 IU/wk for at least 8 doses or equivalent;

### OR

Darbepoetin alpha ≥500 μg Q3W for at least 4 doses or equivalent.

Intolerant to prior ESA treatment—documentation of discontinuation of prior ESA containing regimen, either as single agent or combination (e.g., with G-CSF), at any time after introduction due to intolerance or an adverse event Arm 2

ESA naïve or ineligible—Low chance of response to ESA based on endogenous serum erythropoietin level >200 U/L for subjects not previously treated with ESAs

### Phase 1 and 2

### Exclusion Criteria:

Subjects meeting any one of these exclusion criteria will be prohibited from participating in the study:

1. Presence of concomitant severe cardiovascular disease.
2. Subjects who had myocardial infarction or congestive heart failure within 6 months (180 days) before enrollment.
3. Presence of concomitant malignancy requiring chemotherapy or any malignancy (except basal and squamous cell carcinoma of the skin) for which the patient received chemotherapy within 6 months prior to enrollment. NOTE: Diagnosis of any previous or concomitant malignancy is thus not an exclusion criterion.
4. Uncontrolled systemic fungal, bacterial, or viral infection (defined as ongoing signs/symptoms related to the infection without improvement despite appropriate antibiotics, antiviral therapy, and/or other treatment), known Human Immunodeficiency Virus (HIV), active Hepatitis B Virus (HBV) infection, and/or Hepatitis C (HCV) Infection.
5. Presence of any psychological, familial, sociological or geographical condition that, in the opinion of the investigator, could potentially hinder compliance with the study protocol and follow-up schedule.
6. Subjects with active autoimmune disease who require long-term systemic steroid therapy greater than the equivalent of 20 mg of prednisone daily.
7. Subjects with clinically active uncontrolled, bleeding in the past month.
8. Thrombocytopenia (platelet count <50,000/μL [50×109/L]).
9. Neutropenia (absolute neutrophil count [ANC]<500/μL [0.5×10⁹/L]). Experienced thrombosis ≤6 months prior to enrollment.
10. Women who are pregnant or breastfeeding.
11. Male subjects with partners of childbearing potential who are unwilling to use condoms in combination with a second effective method of contraception during the trial and for 7 months after the last administration of study treatment.
12. Subjects who are unwilling or unable to comply with procedures required in this protocol.
13. Have undergone recent surgery with potential to cause the impairment of gastrointestinal tract absorption or that could cause short bowel syndrome with diarrhea due to malabsorption.
14. Have known hemochromatosis at baseline or a family history of hemochromatosis.

### Investigational Product, Dosage, and Mode of Administration:

### Phase 1-Dose Escalation:

The compound of structure (I), oral, 20 mg daily starting on Cycle 1 Day 1.

Subsequent planned escalation levels for the compound of structure (I) doses are included in Table 1.

Study drug should be taken in the morning after an overnight fast with up to 200 mL or 7 ounces of water at least 1 hour before ingesting any food or other medications.

Phase 2—Dose Expansion:

The compound of structure (I), oral, recommended dose for Phase 2

Statistical methods:

### Phase 1

Two-parameter Bayesian logistic regression model (BLRM) with EWOC will be used to guide dose escalation and estimate the MTD based on occurrence of DLT during Cycle 1. MTD with estimated posterior probability of a DLT within target toxicity interval (16%, 33%) among the admissible doses fulfilling EWOC is determined by BLRM. MTD is estimated based on observed DLTs.

After completing a given dose escalation cohort, the decision to move up to a planned cohort or not, or to adjust to a lower or slightly higher dose, will be decided based on BLRM with EWOC and integrate all available safety data, PK and other clinical data using the BLRM method.

A SRC will consist of the Principal Investigators, an independent cardiologist, the Safety Physician, the Statistician and the Medical Monitor. The SRC will conduct scheduled meetings and will provide safety oversight of the subjects, determine DLTs, and guide escalation and dose decisions. The SRC will meet after all subjects in the newly escalated cohort have had completed the DLT evaluation period and before proceeding with the next cohort at a higher dose level. The SRC will review and assess all available safety data from each cohort, together with available PK and pharmacodynamic data, to determine the escalation to the next dose level cohort. The SRC will also conduct unscheduled meetings on an as needed basis to review other information that may be relevant to the conduct of this study or safety of the subjects.

If a different dose is recommended by the BLRM method and confirmed by the SRC, then enrollment into the next dose level may be initiated.

If a different dose is recommended by the BLRM method versus the dose decided upon by the SRC, further discussions regarding future cohort dosing, will ensue between the SRC and the compound of structure (I) clinical/safety teams in order to make a final decision.

Additional or intermediate dose levels may be explored. It is possible that a potential arm with different dosing schedule is added during the Phase I study period based on safety considerations.

Determination of Recommended Dose:

The recommended dose is usually the highest dose with acceptable toxicity, generally defined as the dose level producing a DLT rate within 16% to 33%. Determination of the recommended dose will be performed in consultation with the SRC based on safety and other data available at the time of the recommended dose decision.

Once the recommended dose for the expansion arms is identified, the Phase 1 portion of the study will progress to Phase 2.

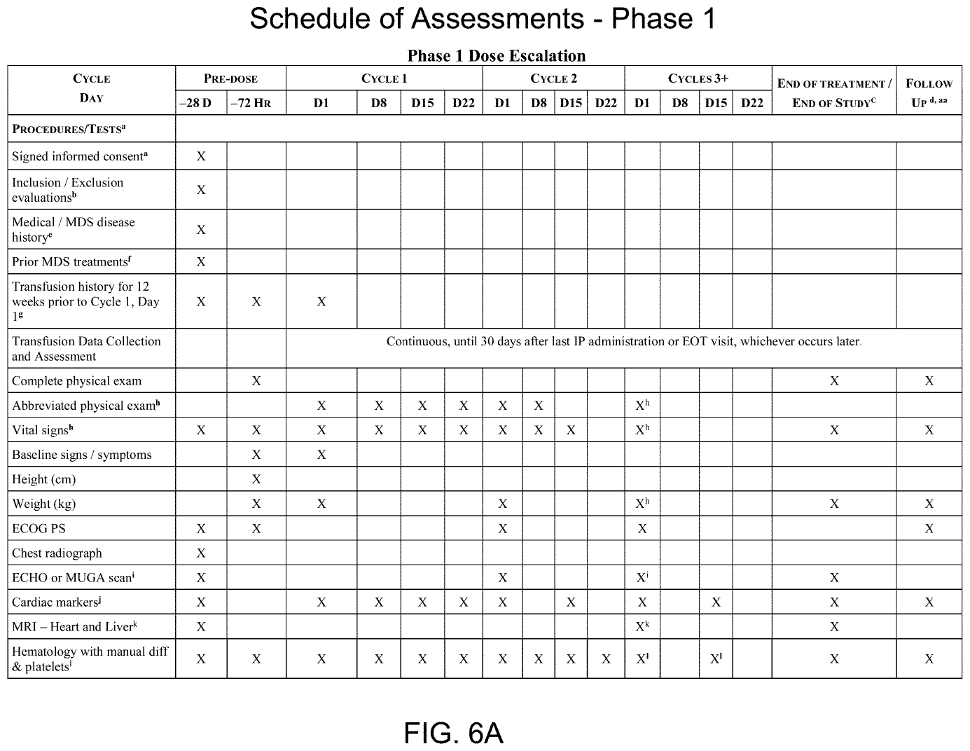

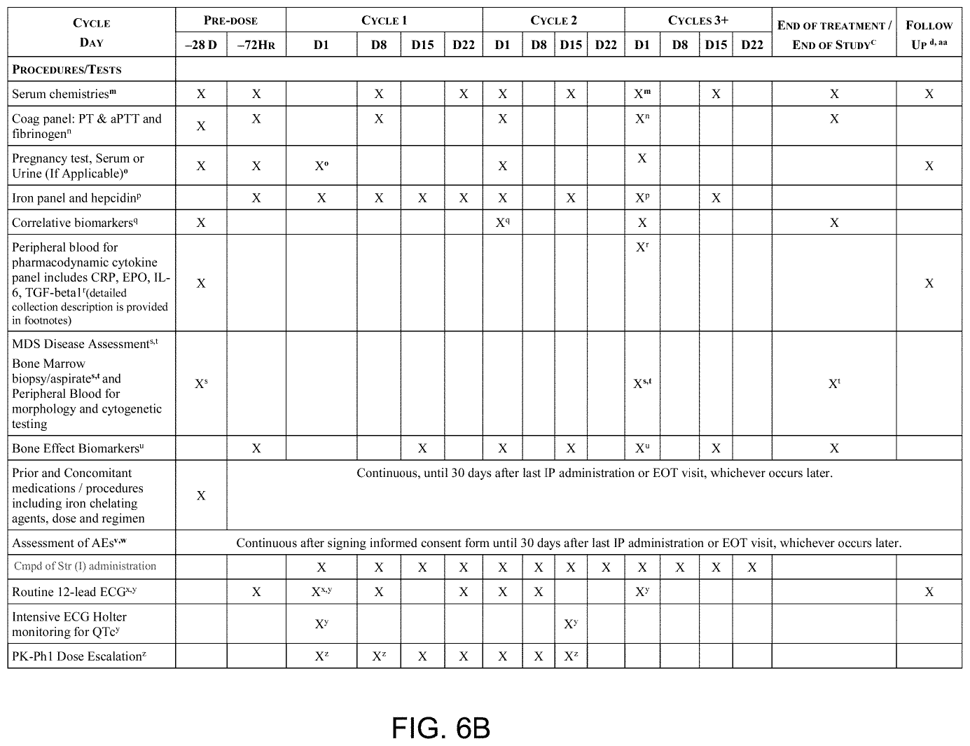

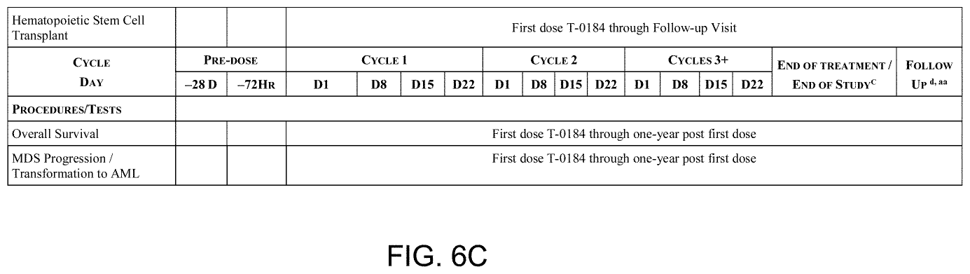

FIG. 6 provides Table 2: Schedule of Assessments—Phase 1

Description of the Figure—Phase 1 Notes:
a. Written informed consent must be obtained prior to conduct of screening evaluations. Window of +/−3 days is allowed for procedures and tests.
b. Review all inclusion and exclusion criteria to determine if patient has met all eligibility criteria for enrollment into the study obtain Medical Monitor (or designee) approval to enroll patient.
c. If, at any time, a patient discontinues study treatment, an End of Treatment visit should be scheduled as soon as possible and within 14 days of the last dose of study drug or within 14 days of the decision to discontinue study treatment. If the decision to withdraw the patient occurs at a regularly scheduled visit, that visit may become the End of Study visit rather than having the patient return for an additional visit.
d. Patients must have a safety evaluation 30 days after the last dose of study drug.
e. Collect and document a complete medical and disease history including initial histologically confirmed and current diagnosis of MDS.
f. Provide all prior treatments for MDS, including ESA, HMAs, EMAs (erythroid maturation agents), ImiDs, and/or investigational drugs.
g. Collect and document complete transfusion history for a minimum of 12 weeks immediately preceding the first dose of the compound of structure (I). This transfusion data must include hemoglobin measured prior to transfusion (pre-transfusion Hgb).
h. Vital signs to include: temperature, heart rate, systolic and diastolic blood pressures, respiration. Abbreviated physical exam may be performed if AE- or symptom-directed. Perform on Day 1 from Cycle 4 onward, including weight.
i. Echocardiograms or MUGA scans—pre-dose, Cycle 2 Day 1, Cycle 6 Day 1, then every 3rd Cycle Day 1 (Cycle 9, 12, etc.) and at EOT; may also be repeated as clinically necessary.
j. Cardiac markers—i.e. B-type natriuretic peptide [BNP], N-terminal pro B-type natriuretic peptide [NT proBNP])—pre-dose, Cycle 1—weekly, Cycle 2 and 3—Day 1 and 15 (biweekly), Cycle 4+Day 1 (every 4 weeks), EOT and 30 days following last dose of the compound of structure (I).
k. MRI of heart and liver to assess iron deposition to be performed pre-study, at Cycle 4 Day 1 (Week 12), Cycle 7 Day 1 (Week 24) and EOT.
l. Hematology assessment (e.g., red blood cell [RBC] count, complete blood count [CBC], white blood cell [WBC] with differential, hemoglobin, hematocrit, nucleated red blood cells [nRBC], absolute reticulocyte count, platelet count, mean corpuscular volume [MCV], mean corpuscular hemoglobin [MCH], mean corpuscular hemoglobin concentrations [MCHC], and red blood cell distribution width [RDW]), weekly from Cycle Day 1 to Cycle 3 Day 1. Then starting at Cycle 3, every Cycle Day 1 and Day 15, EOT and at post-treatment visit.
m. Full serum chemistry panel to include: blood urea nitrogen, phosphorus, magnesium, lactate dehydrogenase, creatinine, uric acid, total protein, albumin, calcium, glucose, total bilirubin, direct bilirubin, alkaline phosphatase, aspartate aminotransferase, alanine aminotransferase, and electrolytes (sodium, potassium, chloride, CO2). Cycle 1-weekly, Cycle 2 biweekly, staring at Cycle 3, every Cycle Day 1, EOT and at post-treatment visit.
n. Coagulation panel [PT & aPTT] and fibrinogen from Cycle 3 onward Day 1 of every Cycle and EOT.
o. Pregnancy testing is performed at the screening visit and will be repeated at subsequent cycles and discontinuation, per institutional standard of care, for women of childbearing potential only. Repeat pregnancy testing if required screening pregnancy test was performed >72 hours prior to first dose.
p. Iron panel (serum iron, ferritin, transferrin, soluble transferrin [STR], and TIBC), including hepcidin—pre-dose, Cycle 1—weekly, Cycle 2 and 3—biweekly (D1 and D15), Cycle 4+D1—every 4 weeks through Cycle 7 D1 (Week 24). Note: Ferritin will be used as a safety parameter.
q. Correlative biomarkers—serum/plasma and bone marrow aspirate (when performed for MDS assessments)-pre-dose, Cycle 2 Dayl and Cycle 3, prior to Cycle 4 Day 1, end of Cycle 6, prior to Cycle 7 Day 1, every 3 Cycles thereafter (end of Cycle 9, end of Cycle 12, etc.) and at EOT.
r. Pharmacodynamic assessments and timepoints:
   Cytokine panel, including CRP, EPO, IL-6, TGF-beta1 in serum and/or plasma—pre-dose, Cycle 4 Dayl (Week 12), Cycle 7 Day 1 (Week 24), every 3 Cycles Day 1 thereafter (Cycle 10, etc.) and EOT.
   Signal transduction pathways inhibited by the compound of structure (I), including phosphorylation of SMAD-1, 2, 3, 5 and 8, in PBMCs and bone marrow aspirates:
      i. PBMC's—pre-dose, Cycle 1—weekly, Cycle 2 and 3—biweekly, Cycle 4—every 4 weeks through Week 24.
      ii. Bone marrow aspirate (when performed for MDS assessments)-screening, end of Cycle 3, end of Cycle 6 and every 3 Cycles thereafter (end of Cycle 9, end of Cycle 12, etc.) and at EOT.
   Gene mutations associated with MDS and/or associated with signal transduction pathways inhibited by the compound of structure (I) in bone marrow aspirates and/or peripheral blood samples—screening and at EOT.
s. Perform bone marrow biopsy and/or aspiration and collect peripheral blood for disease status, standard cytogenetics, assessment of potential biomarkers. If the bone marrow biopsy and/or aspirate is nonproductive or not diagnostic, the procedure must be repeated within 7 days. Six to 8 bone marrow slides will be prepared (in addition to fresh bone marrow samples) and sent to the TBD. Bone marrow biopsy/aspirate performed ≤12 weeks prior to baseline will not need to be repeated if results and minimum slides are available. If >12 weeks since last bone marrow response assessment, perform bone marrow biopsy and aspiration and collect peripheral blood sample for assessment of response (Appendix 3) and potential biomarkers.
t. Response assessments include hematology and bone marrow biopsy/aspirate and should be repeated end of Cycle 3, end of Cycle 6, and every 3 Cycles thereafter (end of Cycle 9, end of Cycle 12, etc.) and at EOT. If medically appropriate, response assessments should be repeated at the time of MDS progression and/or as clinically indicated.
u. Bone Effect Biomarkers—Bone specific alkaline phosphatase (BSALP), C-terminal and N-terminal Type 1 Collagen Telopeptide (CTX/NTX) in serum, performed pre-dose, C1D15, Cycle 2 and 3 biweekly and every Cycle Dayl thereafter and EOT.
v. Toxicities will be assessed according to the NCI CTCAE v5.0 (see Appendix 3). When the NCI CTCAE grade is not available, the investigator will use the following toxicity grading: mild, moderate, severe, life-threatening or fatal.
w. Ongoing AEs must be followed clinically until the event is resolved, deemed permanent or no longer clinically significant, or the patient begins an alternative treatment regimen.
x. Routine 12-lead ECG- to be performed on PK sampling days prior to pre-dose the compound of structure (I) PK blood collection and at every subsequent Cycle on Day 1. Routine ECG will not be performed on visit days that intensive Holter monitoring is performed. Note: ECGs should be performed prior to any blood sampling.
y. Intensive ECG Holter monitoring, including assessment of QTcF, to be conducted at first dose of the compound of structure (I), Cycle D1 through D2 and at steady state, Cycle 2 D15 through D16. Note: Intensive Holter monitoring should be started prior to any blood sampling.

Phase 2

The Phase 2 design is based on Bayesian efficacy monitoring using posterior probability criteria.

Approximately 20-40 subjects will be enrolled into each of the treatment Arms (Arm 1 or Arm 2). After the first 10 subjects are evaluable for efficacy (Efficacy Set) in the treatment arm (Arm1 or Arm 2), an interim efficacy monitoring using Bayesian posterior probability will be performed. Enrollment may be stopped early due to futility or early efficacy if the Bayesian posterior probability meets the early efficacy or futility criteria.

The primary endpoint of Phase 2 portion is response rate based on an efficacy composite endpoint, the objective, components, and assessment time are included in FIG. 1 below. This composite endpoint will be used for response rate evaluation and analyzed using Bayesian efficacy monitoring.

### DLT Definition

A DLT is defined as any one of the following events or abnormal laboratory value not clearly unrelated to the study drug observed within 28 days of starting treatment with the compound of structure (I):
Any death not clearly due to the underlying progression of disease (such as leukemic transformation) or co-morbid medical conditions.
Absolute neutrophil count (ANC) not recovering to >500/μL within 14 days in the absence of myelodysplasia or transformation to acute leukemia (To determine progression of MDS/transformation to AML, a bone marrow biopsy/aspiration and/or peripheral blood can be performed).
Platelet count not recovering to >25,000/μL within 14 days in the absence of myelodysplasia or transformation to acute leukemia. (To determine progression of MDS/transformation to AML, a bone marrow biopsy/aspiration and/or peripheral blood can be performed.)
Any grade 4 febrile neutropenia.
Persistent Grade 3 or 4 nausea, vomiting, or diarrhea >72 hours despite adequate prophylactic and supportive care.
Grade 4 non-hematologic toxicity.
Any AST and ALT elevation >3×ULN accompanied by serum bilirubin >2× ULN without initial findings of cholestasis (elevated serum alkaline phosphatase), and no other reason can be found to explain the combination of increased aminotransferases and total bilirubin, such as viral hepatitis A, B, or C; preexisting or acute liver disease; or another drug capable of causing the observed injury.
New onset symptomatic Class III cardiac failure based on NYHA Functional Classification.
ECHO or MUGA reduction of ejection fraction ≥10%.
Serum Ferritin increase by 1,000 ng/mL from baseline level.

Note: As neutropenic and thrombocytopenic subjects are included in the study; any Grade 3 neutropenia <1000/uL neutrophils, and grade three bleeding with <50 K platelets in subjects with prior history of specific chronic neutropenia with infection, or thrombocytopenia from specific area of bleeding, will be evaluated by the SRC in consideration as to whether a DLT has occurred. In the situation where the Grade 3 AE is thought by the SRC to be related only to the biology of MDS, no DLT will be declared.

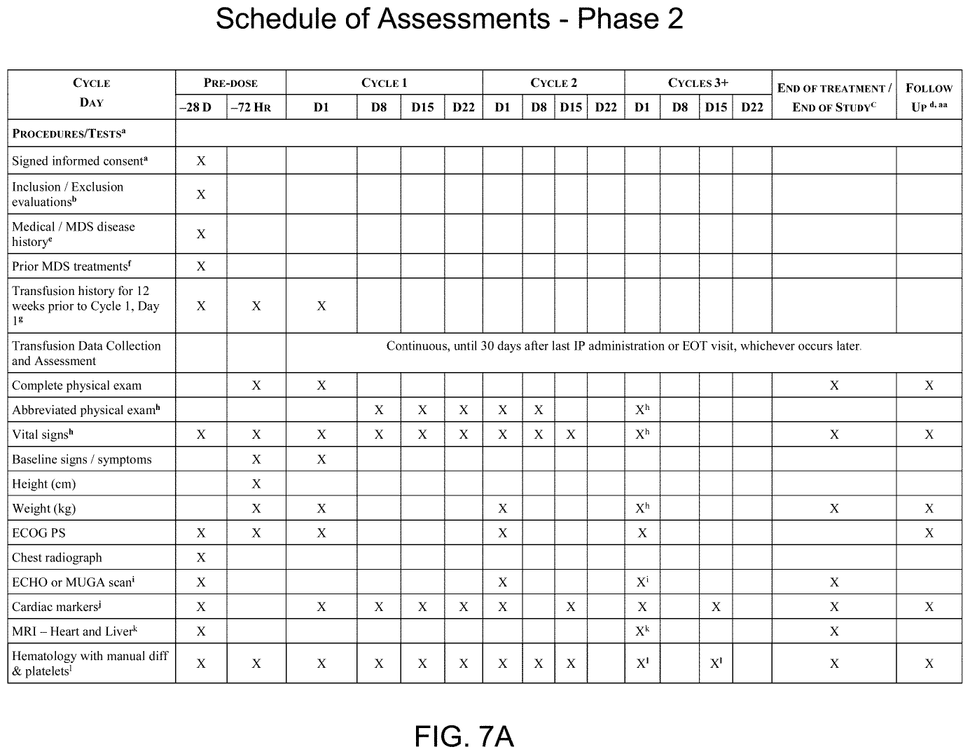

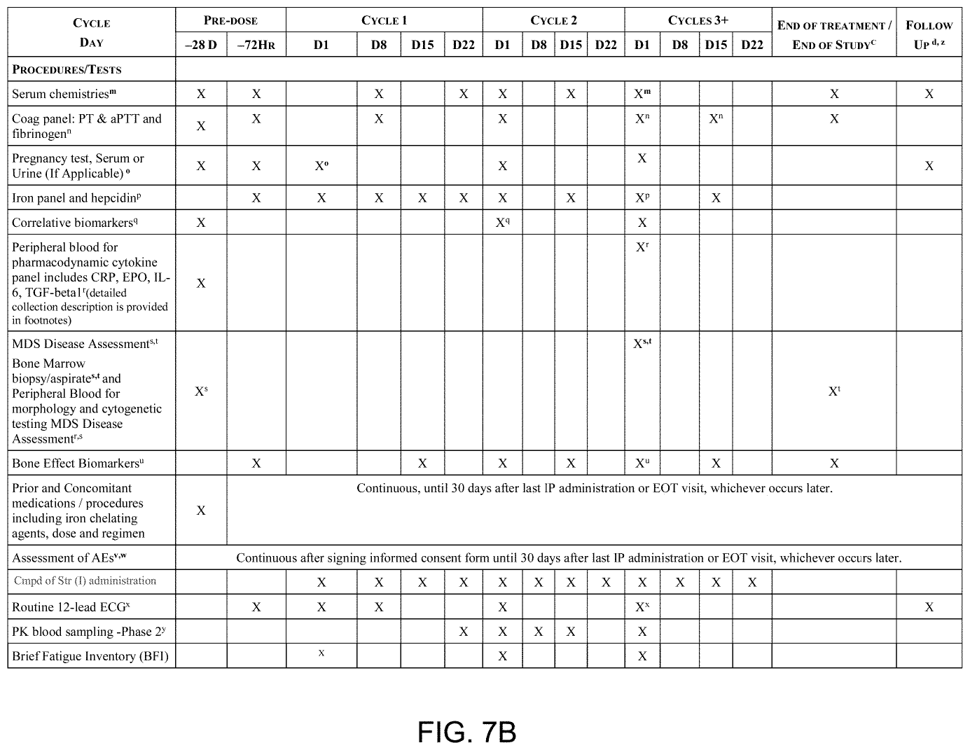

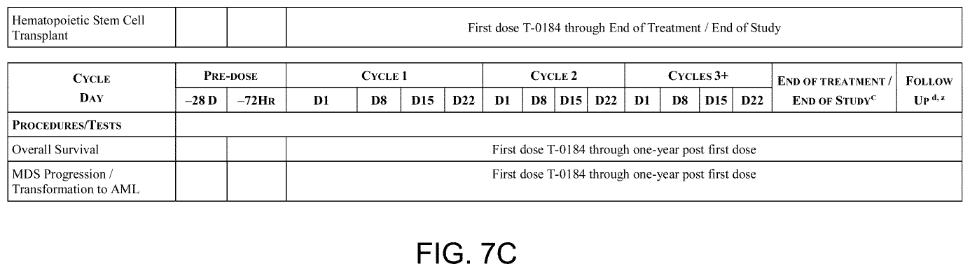

FIG. 7 provides Table 3: Schedule of Assessments—Phase 2

Description of the Figure—Phase 2 Notes:
a. Written informed consent must be obtained prior to conduct of screening evaluations. Window of +/−3 days is allowed for procedures and tests.
b. Review all inclusion and exclusion criteria to determine if patient has met all eligibility criteria for enrollment into the study. obtain Medical Monitor (or designee) approval to enroll patient.
c. If, at any time, a patient discontinues study treatment, an End of Treatment visit should be scheduled as soon as possible and within 14 days of the last dose of study drug or within 14 days of the decision to discontinue study treatment. If the decision to withdraw the patient occurs at a regularly scheduled visit, that visit may become the End of Study visit rather than having the patient return for an additional visit.
d. Patients must have a safety evaluation 30 days after the last dose of study drug.
e. Collect and document a complete medical and disease history including initial histologically confirmed and current diagnosis of MDS.
f. Provide all prior treatments for MDS, including ESA, HMAs, EMAs (erythroid maturation agents), ImiDs, and/or investigational drugs.
g. Collect and document complete transfusion history for a minimum of 12 weeks immediately preceding the first dose of the compound of structure (I). This transfusion data must include hemoglobin measured prior to transfusion (pre-transfusion Hgb).
h. Vital signs to include: temperature, heart rate, systolic and diastolic blood pressures, respiration. Abbreviated physical exam may be performed if AE- or symptom-directed. Perform on Day 1 from Cycle 4 onward, including weight.
i. Echocardiograms or MUGA scans—pre-dose, Cycle 2 Day 1, Cycle 6 Day 1, then every 3rd Cycle Day 1 (Cycle 9, 12, etc.) and at EOT; may also be repeated as clinically necessary.
j. Cardiac markers—i.e. B-type natriuretic peptide [BNP], N-terminal pro B-type natriuretic peptide [NT proBNP])—pre-dose, Cycle 1—weekly, Cycle 2 and 3—Day 1 and 15 (biweekly), Cycle 4+Day 1 (every 4 weeks), EOT and 30 days following last dose of the compound of structure (I).
k. Mill of heart and liver to assess iron deposition to be performed pre-study, at Cycle 4 Day 1 (Week 12), Cycle 7 Day 1 (Week 24) and EOT.
l. Hematology assessment (e.g., red blood cell [RBC] count, complete blood count [CBC], white blood cell [WBC] with differential, hemoglobin, hematocrit, nucleated red blood cells [nRBC], absolute reticulocyte count, platelet count, mean corpuscular volume [MCV], mean corpuscular hemoglobin [MCH], mean corpuscular hemoglobin concentrations [MCHC], and red blood cell distribution width [RDW]), weekly from Cycle Day 1 to Cycle 3 Day 1. Then starting at Cycle 3, every Cycle Day 1 and Day 15, EOT and at post-treatment visit.
m. Full serum chemistry panel to include: blood urea nitrogen, phosphorus, magnesium, lactate dehydrogenase, creatinine, uric acid, total protein, albumin, calcium, glucose, total bilirubin, direct bilirubin, alkaline phosphatase, aspartate aminotransferase, alanine aminotransferase, and electrolytes (sodium, potassium, chloride, CO2). Cycle 1-weekly, Cycle 2 biweekly, staring at Cycle 3, every Cycle Day 1, EOT and at post-treatment visit.
n. Coagulation panel [PT & aPTT] and fibrinogen from Cycle 3 onward Day 1 of every Cycle and EOT.
o. Pregnancy testing is performed at the screening visit and will be repeated at subsequent cycles and discontinuation, per institutional standard of care, for women of childbearing potential only. Repeat pregnancy testing if required screening pregnancy test was performed >72 hours prior to first dose.
p. Iron panel (serum iron, ferritin, transferrin, soluble transferrin [STR], and TIBC), including hepcidin—pre-dose, Cycle 1—weekly, Cycle 2 and 3—biweekly (D1 and D15), Cycle 4+D1—every 4 weeks through Cycle 7 D1 (Week 24). Note: Ferritin will be used as a safety parameter.
q. Correlative biomarkers—serum/plasma and bone marrow aspirate (when performed for MDS assessments)-pre-dose, Cycle 2 Dayl and Cycle 3, prior to Cycle 4 Day 1, end of Cycle 6, prior to Cycle 7 Day 1, every 3 Cycles thereafter (end of Cycle 9, end of Cycle 12, etc.) and at EOT.
r. Pharmacodynamic assessments and timepoints:
   Cytokine panel, including CRP, EPO, IL-6, TGF-beta1 in serum and/or plasma—pre-dose, Cycle 4 Dayl (Week 12), Cycle 7 Day 1 (Week 24), every 3 Cycles Day 1 thereafter (Cycle 10, etc.) and EOT.
   Signal transduction pathways inhibited by the compound of structure (I), including phosphorylation of SMAD-1, 2, 3, 5 and 8, in PBMCs and bone marrow aspirates:
      i. PBMC's—pre-dose, Cycle 1—weekly, Cycle 2 and 3—biweekly, Cycle 4—every 4 weeks through Week 24.
      ii. Bone marrow aspirate (when performed for MDS assessments)-screening, end of Cycle 3, end of Cycle 6 and every 3 Cycles thereafter (end of Cycle 9, end of Cycle 12, etc.) and at EOT.
s. Gene mutations associated with MDS and/or associated with signal transduction pathways inhibited by the compound of structure (I) in bone marrow aspirates and/or peripheral blood samples—screening and at EOT.
t. Perform bone marrow biopsy and/or aspiration and collect peripheral blood for disease status, standard cytogenetics, assessment of potential biomarkers. If the bone marrow biopsy and/or aspirate is nonproductive or not diagnostic, the procedure must be repeated within 7 days. Six to 8 bone marrow slides will be prepared (in addition to fresh bone marrow samples) and sent to the TBD. Bone marrow biopsy/aspirate performed ≤12 weeks prior to baseline will not need to be repeated if results and minimum slides are available. If >12 weeks since last bone marrow response assessment, perform bone marrow biopsy and aspiration and collect peripheral blood sample for assessment of response (Appendix 3) and potential biomarkers.
u. Response assessments include hematology and bone marrow biopsy/aspirate and should be repeated end of Cycle 3, end of Cycle 6, and every 3 Cycles thereafter (end of Cycle 9, end of Cycle 12, etc.) and at EOT. If medically appropriate, response assessments should be repeated at the time of MDS progression and/or as clinically indicated.
v. Bone Effect Biomarkers—Bone specific alkaline phosphatase (BSALP), C-terminal and N-terminal Type 1 Collagen Telopeptide (CTX/NTX) in serum, performed pre-dose, C1D15, Cycle 2 and 3 biweekly and every Cycle Dayl thereafter and EOT.
w. Toxicities will be assessed according to the NCI CTCAE v5.0 (see Appendix 3). When the NCI CTCAE grade is not available, the investigator will use the following toxicity grading: mild, moderate, severe, life-threatening or fatal.
x. Ongoing AEs must be followed clinically until the event is resolved, deemed permanent or no longer clinically significant, or the patient begins an alternative treatment regimen.
y. Routine 12-lead ECG- to be performed on Day 1 of each Cycle. Note: ECGs should be performed prior to any blood sampling.

### Example 7

### Salt Evaluation and Polymorph Screen

A salt screening evaluated the basic compound, the compound of structure (I), to assess whether a salt form would provide benefits over the freebase form. For any suitable salt candidate identified, a preliminary polymorph screening would be performed to evaluate its polymorphism risk.

### Summary

Salt screening was performed under 33 conditions using 10 acids (two molar ratios of HCl) and three solvent systems. From all the screening experiments, a total of 12 crystalline hits were isolated and characterized by X-ray powder diffraction (XRPD), thermo-gravimetric analysis (TGA), and differential scanning calorimetry (DSC). The stoichiometric ratio of salt hits was determined by proton nuclear magnetic resonance (1H NMR) or high-performance liquid chromatography (HPLC) combined with ion chromatography (IC). Based on the physical properties of the hits, anhydrous HCl salt Form A was selected as the salt lead for evaluation.

The salt lead of HCl salt Form A was prepared to 300 mg scale and evaluated on hygroscopicity, kinetic solubility in pH 2, 5, and 7 buffers, and solid-state stability under 40° C./75% RH for one week. As shown by the evaluation results (using freebase Form A as reference):
a) Freebase Form A, and HCl salt Form A were slightly hygroscopic with no form change after DVS tests;
b) Compared with freebase Form A, HCl salt Form A showed increased solubility in pH 2, 5 and 7 buffers, and disproportionation was observed in pH 7 buffer; and
c) Freebase Form A and HCl salt Form A showed good physicochemical properties under 40° C./75% RH for one week. The characterization and evaluation results are summarized in Table 4.

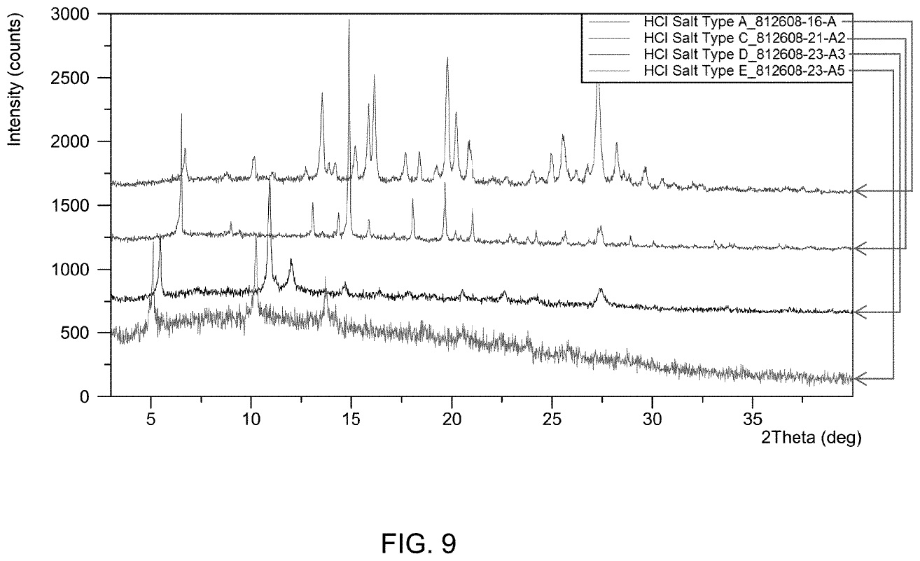

Based on results collected, HCl salt Form A is a preferred candidate form. Therefore, a polymorphism evaluation study was performed on the HCl salt (mono). Starting with HCl salt Form A, a preliminary polymorph screening was conducted under 32 conditions using different methods of slurry conversion, evaporation, slow cooling and anti-solvent addition. Based on investigation results, HCl salt Form A is speculated to be anhydrate and hydrate, respectively. Detailed characterization data and XRPD overlay of HCl salt forms obtained from both salt and polymorph screening are summarized in Table 5A, FIG. 8, and FIG. 9. FIG. 8 depicts an XRPD pattern of HCl salt Form A. FIG. 9 depicts an overlay of HCl salt crystal forms A, C, D, and E. Each form may also be referred to as a “type” and the terms are used interchangeably.

TABLE 4
Characterization and evaluation summary of salt leads and freebase

| Salt Form | Freebase Form A | HCl Salt Form A |
|---|---|---|
| (Batch No.) | (812608-05-A) | (812608-12-A) |
| Stoichiometry | — | 0.97 (by IC/HPLC) |
| (acid/freebase) | | |
| Safety Class of | — | I |
| Acid | | |
| HPLC Purity | 99.17 | 99.63 |
| (area %) | | |
| Speculated Form | Anhydrate | Anhydrate |
| Weight Loss (%) | 1.8 | 1.7 |
| Endotherm (° C., | 200.2 | 198.9*, 218.0, 275.9 |
| peak) | | |
| Hygroscopicity | Slightly hygroscopic | Slightly hygroscopic |
| (%)** | (0.60) | (0.86) |

| | |
|---|---|
| Kinetic Solubility | Compared with freebase Form A, |
| | HCl Form A showed increased solubility |
| | in pH 2, 5 and 7 buffers; |
| Solid-state | Good physicochemical properties under |
| Stability | 40° C./75% RH for at least one week. |

—: not available.
*might be caused by a very small amount of freebase Form A remaining.
**based on water uptake at 25° C./80% RH: very hygroscopic - >15%, hygroscopic - 2~15%, slightly hygroscopic - 0.2~2%, non-hygroscopic - <0.2%.

TABLE 5A
Characterization of HCl salt forms

| | | Weight | Endotherm | | |
|---|---|---|---|---|---|
| Crystal | Sample | Loss in | in DSC | Stoichiometry | Speculated |
| Form | ID | TGA (%) | (° C., peak) | (acid/FB)* | Form |
| Form A | 812608- | 2.0 | 223.5, 276.4 | 1.01 | Anhydrate |
| | 16-A | | | | |

As noted, FIG. 8 depicts an XRPD pattern of HCl salt Form A. A tabulated version of the XRPD for Form A is as follows in Table 5B, noting an error range+/− of about 0.2° 2θ as appreciated by those skilled in the art:

TABLE 5B
The compound of structure (I) HCl salt Form A

| Pos. [°2θ] | Height [cts] | Area [cts °2θ] | d-spacing [Å] | Rel. Int. [%] |
|---|---|---|---|---|
| 3.9059 | 298.79 | 37.70 | 22.62205 | 2.85 |
| 6.7125 | 2201.43 | 222.23 | 13.16854 | 20.97 |
| 8.8145 | 454.58 | 51.63 | 10.03229 | 4.33 |
| 10.1413 | 1672.42 | 211.04 | 8.72261 | 15.93 |
| 11.0558 | 561.23 | 70.82 | 8.00302 | 5.35 |
| 12.7048 | 1080.46 | 136.34 | 6.96775 | 10.29 |
| 13.5347 | 6642.71 | 922.04 | 6.54234 | 63.28 |
| 13.8769 | 1131.77 | 142.81 | 6.38178 | 10.78 |
| 14.1755 | 1620.14 | 224.88 | 6.24800 | 15.43 |
| 15.1840 | 3546.33 | 537.00 | 5.83520 | 33.78 |
| 15.8491 | 5585.02 | 775.23 | 5.59182 | 53.21 |
| 16.1455 | 8599.25 | 1193.62 | 5.48981 | 81.92 |
| 17.2245 | 162.88 | 26.72 | 5.14828 | 1.55 |
| 17.6770 | 2882.25 | 436.44 | 5.01749 | 27.46 |
| 18.3807 | 2578.71 | 390.48 | 4.82696 | 24.57 |
| 19.2499 | 1100.51 | 166.64 | 4.61093 | 10.48 |
| 19.7721 | 9580.62 | 1450.74 | 4.49032 | 91.27 |
| 20.2054 | 5072.51 | 896.12 | 4.39499 | 48.32 |
| 20.8140 | 3168.56 | 399.83 | 4.26782 | 30.19 |
| 20.9432 | 2000.42 | 227.18 | 4.24178 | 19.06 |
| 22.0018 | 366.67 | 64.72 | 4.04003 | 3.49 |
| 22.6847 | 604.86 | 76.32 | 3.91994 | 5.76 |
| 23.9816 | 1024.54 | 181.00 | 3.71080 | 9.76 |
| 24.4538 | 531.66 | 80.51 | 3.64021 | 5.06 |
| 24.9644 | 2159.45 | 326.99 | 3.56690 | 20.57 |
| 25.5118 | 4541.08 | 802.23 | 3.49160 | 43.26 |
| 26.1922 | 763.31 | 86.69 | 3.40242 | 7.27 |
| 26.7501 | 1326.52 | 200.87 | 3.33272 | 12.64 |
| 27.2385 | 10497.03 | 1854.42 | 3.27406 | 100.0 |
| 28.1817 | 2992.14 | 528.60 | 3.16659 | 28.50 |
| 28.5514 | 789.98 | 109.65 | 3.12642 | 7.53 |
| 28.8497 | 599.52 | 90.78 | 3.09477 | 5.71 |
| 29.6008 | 1378.42 | 260.91 | 3.01793 | 13.13 |
| 30.4479 | 633.83 | 103.97 | 2.93586 | 6.04 |
| 31.0671 | 347.19 | 52.57 | 2.87875 | 3.31 |
| 31.9977 | 365.28 | 92.19 | 2.79712 | 3.48 |
| 32.4347 | 376.35 | 80.73 | 2.76042 | 3.59 |
| 33.3026 | 174.88 | 26.48 | 2.69045 | 1.67 |
| 33.6159 | 293.68 | 44.47 | 2.66608 | 2.80 |
| 34.0326 | 123.70 | 18.73 | 2.63439 | 1.18 |
| 34.7780 | 211.12 | 42.62 | 2.57961 | 2.01 |
| 35.5705 | 123.93 | 25.02 | 2.52394 | 1.18 |
| 36.6319 | 310.89 | 86.31 | 2.45321 | 2.96 |
| 37.4707 | 246.78 | 43.60 | 2.40020 | 2.35 |
| 38.1695 | 235.29 | 71.26 | 2.35785 | 2.24 |
| 40.1011 | 135.41 | 34.18 | 2.24862 | 1.29 |
| 40.7471 | 678.30 | 77.03 | 2.21445 | 6.46 |
| 41.2836 | 367.58 | 74.21 | 2.18690 | 3.50 |
| 41.9985 | 284.08 | 64.53 | 2.15132 | 2.71 |
| 43.6795 | 124.43 | 31.40 | 2.07234 | 1.19 |
| 44.5269 | 137.92 | 34.81 | 2.03485 | 1.31 |
| 45.4240 | 143.20 | 36.14 | 1.99673 | 1.36 |
| 46.4339 | 166.33 | 58.77 | 1.95563 | 1.58 |
| 47.3800 | 176.53 | 53.46 | 1.91877 | 1.68 |

As a result of preliminary salt screening of the compound of structure (I) and polymorph screening of HCl salt (mono), the mono-HCl salt Form A is a preferred candidate for further development.

### Detail: Salt Screening and Lead Re-Preparation

According to estimated pKa values of 7.5 and 5.1 and approximate solubility of freebase (812608-05-A) at room temperature (RT, 25±3° C.), 10 salt formers and three solvent systems were used for the screening. Freebase (˜15 mg) was dispersed with selected solvent in a glass vial and corresponding salt former was added with a molar charge ratio of 1:1 (for HCl/freebase, two ratios of both 1:1 and 2:1 were used). The mixtures of freebase and acid were stirred at RT for 3.5 days. To obtain more solid hits, clear solutions obtained (812608-08-B0/B5/B10) were transferred to 5° C. and stirred for another 2.5 days. Finally, to clear solutions of 812608-08-B0/B10, 0.5 mL of n-heptane was added and stirred at 5° C. for another two days.

All the resulted solids were isolated and analyzed by XRPD after being dried at 50° C. for 2.5 hours. As summarized in Table 6, a total of 12 crystalline hits were obtained and characterized by)(RFD, TGA, and DSC with the stoichiometry determined by 1H NMR or HPLC/IC. The characterization data were summarized in Table 7.

TABLE 6
Summary of salt screening results

| | | Solvent | | |
|---|---|---|---|---|
| | | A | B | C |
| Acid | | EtOH | THF | EtOAc |
| 0 | Blank | FB Form A | FB Form A | FB Form A |
| 1 | HCl (1:1) | HCl salt Form A | HCl salt Form A | HCl salt |
| | | | | Form A |

TABLE 7
Characterization summary of crystalline hits

| | | | | Endotherm | Molar |
|---|---|---|---|---|---|
| | | | Wt Loss | (DSC, ° C., | Ratio |
| Hit | | Sample ID | (TGA, %) | peak) | (acid/base) |
| HCl Salt | Form A | 812608-08-A1 | 1.2 | 227.0, | 1.00 |
| | | | | 276.2 | |
| | Form B | 812608-08-C2 | 8.1 | 102.2, | 1.71 |
| | | | | 144.6, | |
| | | | | 240.0, | |
| | | | | 281.6 | |

### Re-preparation and Characterization of Salt Leads

Based on the characterization results, two salt leads (HCl salt Form A and fumarate Form A) were agreed as salt leads and re-prepared to hundreds of milligrams. The selection criteria include but not limited to: 1) sharp XRPD peaks without apparent amorphous halo, 2) negligible weight loss in TGA, 3) neat thermal event with a sharp melting peak in DSC. The detailed preparation procedures were described in Table 8 and the characterization data were summarized above in Table 4.

TABLE 8
Preparation procedures of salt leads

| Crystal Form | Preparation Procedures |
|---|---|
| HCl Salt | 1. Add 53.2 μL HCl to 5.0 mL EtOH in a 20-mL glass |
| Form A | vial. |
| (812608-12-A) | 2. Weigh 300.2 mg freebase into a 20-mL glass vial, |
| | and add 5.0 mL EtOH. A suspension was obtained. |
| | 3. Pipette the acid stock solution into the 20-mL vial |
| | and magnetically stir at RT. |
| | 4. Add ~5 mg of HCl salt Form A seed (812608-08- |
| | A1). |
| | 5. Sample for XRPD after stirring for 1 day, and the |
| | pattern conformed to HCl salt Form A. |
| | 6. Centrifuge the suspension obtained and dry the wet |
| | cake at 50° C. for 2.5 hrs. |
| | 7. Collect solids of 255.4 mg, with a yield of ~79.8%. |

### HCl Salt Form A

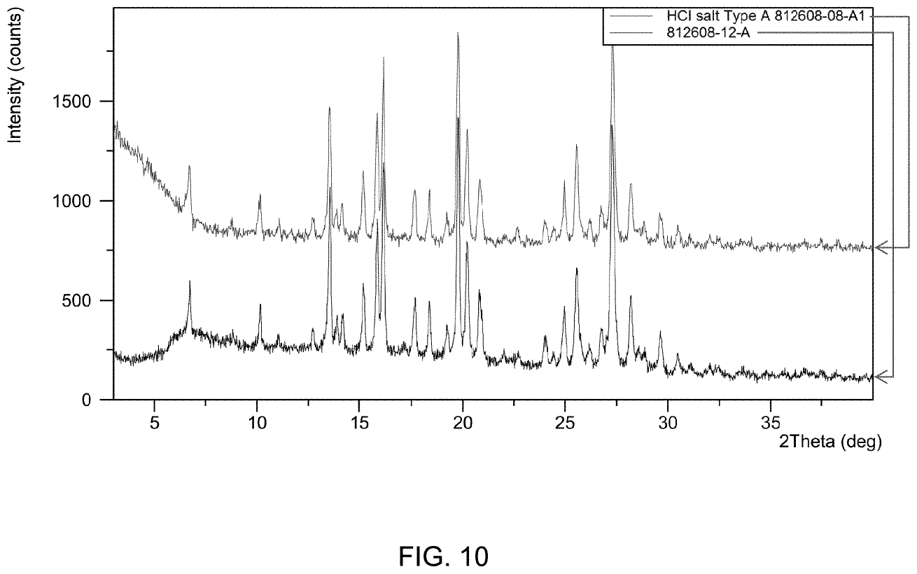

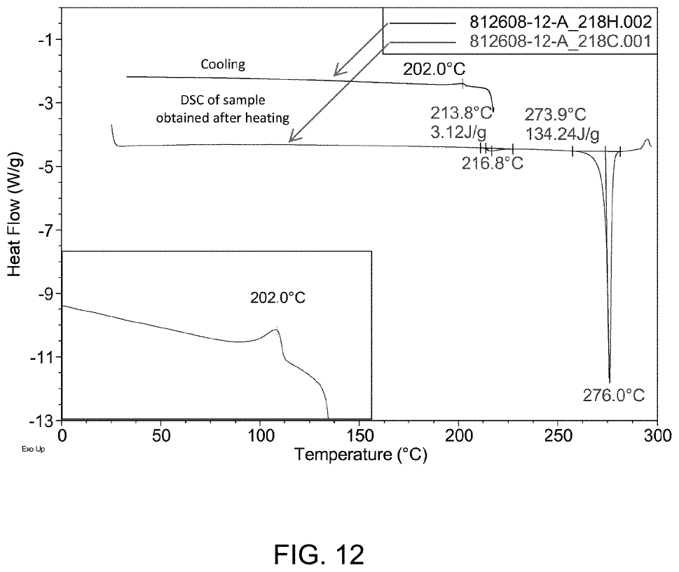

HCl salt Form A was successfully re-prepared as evidenced by XRPD results in FIG. 10. As per TGA and DSC data in FIG. 11, the sample showed a weight loss of 1.7% up to 150° C. and three endotherms at 196.2, 214.8 and 274.0° C. (onset temperature). The small endotherm at 196.2° C. might be caused by the melting of a very small amount of freebase Form A remaining. As shown in FIG. 12, after heating the HCl salt Form A sample to 218° C., an exotherm around 202.0° C. was observed during cooling and DSC of the sample obtained after heating still showed endotherms at 213.8 and 273.9° C. (onset temperature). Combined with the fact that no form change was observed after heating the sample to 218° C., cooling back to RT and exposed to ambient conditions, the signal at 213.8° C. was speculated to be caused by form transition. The stoichiometric ratio was determined as 0.97 (acid/base) by HPLC/IC. As limited gradual TGA weight loss before 150° C. and no significant thermal event in DSC before 190° C. was observed, the sample is speculated to be an anhydrous HCl salt.

### Example 8

### Oral Solid Formulation

The hydrochloride salt of the compound of structure (I) was formulated into three (3) oral dose strengths (5, 25, and 125 mg dose [based on free base]). Increasing amounts of active pharmaceutical ingredient were formulated into three similar blends, see, Table 19. The product was formulated for immediate release using common excipients in the blend. The drug was placed in #3, hard gelatin capsules.

TABLE 9
Excipients in the Blend, 5, 25, and 125 mg Strength Capsules

| Excipient | Purpose |
|---|---|
| Microcrystalline Cellulose | Diluent |
| Lactose Monohydrate | Diluent |
| Croscarmellose Sodium | Disintegrant |
| Magnesium Stearate | Lubricant |

1-107. (canceled)

108. A method for treating anemia in a subject in need thereof comprising administering to the subject an effective amount of a crystalline salt of a compound of structure (I):

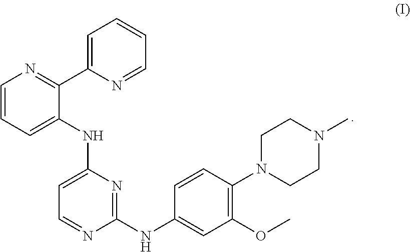 [SMILES: COc1cc(Nc2nccc(Nc3cccnc3-c3ccccn3)n2)ccc1N1CCN(C)CC1]

109. The method of claim 108, wherein the crystalline salt is a hydrochloric acid salt, a monovalent hydrochloric acid salt, an anhydrous hydrochloric acid salt, a Form A hydrochloric acid salt, a Form A hydrochloric acid salt characterized by an x-ray diffraction pattern comprising one or more 20 values selected from 13.53, 16.14, 17.67, 18.38, 24.96 and 28.18, or a Form A hydrochloric acid salt characterized by an x-ray diffraction pattern substantially the same as FIG. 8.

110. The method of claim 108, wherein the crystalline salt of the compound of structure (I) is administered orally at a daily dosage of from 10 mg to 350 mg, from 90 mg to 120 mg, 20 mg, 40 mg, 60 mg, 90 mg, 120 mg, 160 mg, 210 mg, or 270 mg; or is administered orally at a weekly dosage of from 10 mg to 350 mg, from 10 mg to 300 mg, from 30 mg to 90 mg, from 30 mg to 300 mg, from 75 mg to 300 mg, from 85 mg to 300 mg, from 95 mg to 300 mg, from 100 mg to 300 mg, 30 mg, 60 mg, 90 mg, or 120 mg; or is administered orally at a maintenance dosage regime.

111. The method of claim 108, wherein the method improves one or more hematologic parameters in the subject, said improvement comprises decreasing myoblasts, increasing hemoglobin, increasing platelets, increasing neutrophils, decreasing hepcidin, reducing units of red blood cell transfused, reducing frequency of transfusion, and/or reducing transfusion dependence.

112. The method of claim 108, wherein the subject has very low, low or intermediate myelodysplastic syndrome, anemia associated with myelodysplastic syndrome, transfusion dependent anemia associated with myelodysplastic syndrome, myelodysplastic syndrome with single lineage dysplasia refractory anemia, or myelodysplastic syndrome with ring sideroblasts and is intolerant, resistant, or refractory to luspatercept.

113. The method of claim 108, further comprising administering an effective amount of one or more therapeutically active agents.

114. The method of claim 108, wherein the compound of structure (I) is formulated in a gelatin capsule comprising about 5 mg, 25 mg, or 125 mg, which is based on free base weight, of the crystalline salt of the compound of structure (I) and pharmaceutically acceptable carriers comprising microcrystalline cellulose, lactose monohydrate, croscarmellose sodium, magnesium stearate, and a combination thereof.

115. A method for treating anemia in a subject in need thereof comprising administering to the subject an effective amount of a compound of structure (I): 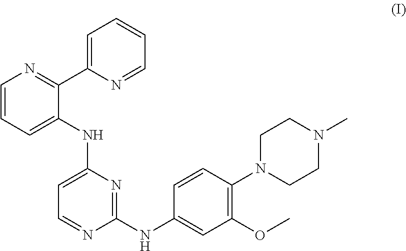 [SMILES: COc1cc(Nc2nccc(Nc3cccnc3-c3ccccn3)n2)ccc1N1CCN(C)CC1]
or a pharmaceutically acceptable salt, or prodrug thereof, wherein the subject has very low, low or intermediate myelodysplastic syndrome (MDS).

116. The method of claim 115, wherein anemia is anemia associated with MDS, transfusion dependent anemia with MDS, MDS with single lineage dysplasia refractory anemia, or MDS with ring sideroblasts and is intolerant, resistant, or refractory to luspatercept.

117. The method of claim 115, wherein the compound of structure (I) is administered orally at a daily dosage of from 10 mg to 350 mg, from 90 mg to 120 mg, 20 mg, 40 mg, 60 mg, 90 mg, 120 mg, 160 mg, 210 mg, or 270 mg; or is administered orally at a weekly dosage of from 10 mg to 350 mg, from 10 mg to 300 mg, from 30 mg to 90 mg, from 30 mg to 300 mg, from 75 mg to 300 mg, from 85 mg to 300 mg, from 95 mg to 300 mg, from 100 mg to 300 mg, 30 mg, 60 mg, 90 mg, or 120 mg; or is administered orally at a maintenance dosage regime.

118. The method of claim 115, wherein the method improves one or more hematologic parameters in the subject, said improvement comprises decreasing myoblasts, increasing hemoglobin, increasing platelets, increasing neutrophils, decreasing hepcidin, reducing units of red blood cell transfused, reducing frequency of transfusion, and/or reducing transfusion dependence.

119. The method of claim 115, further comprising administering an effective amount of one or more therapeutically active agents.

120. The method of claim 115, wherein the compound of structure (I) is formulated in a gelatin capsule comprising about 5 mg, 25 mg, or 125 mg, which is based on free base weight, of the compound of structure (I) and pharmaceutically acceptable carriers comprising microcrystalline cellulose, lactose monohydrate, croscarmellose sodium, magnesium stearate, and a combination thereof.

121. A method for treating myelodysplastic syndrome (MDS) in a subject in need thereof comprising administering to the subject an effective amount of a compound of structure (I): 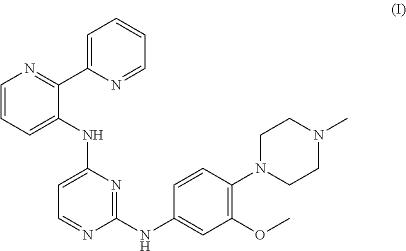 [SMILES: COc1cc(Nc2nccc(Nc3cccnc3-c3ccccn3)n2)ccc1N1CCN(C)CC1]
or a pharmaceutically acceptable salt, or prodrug thereof.

122. The method of claim 121, wherein said MDS is primary MDS, secondary MDS, high-risk MDS, intermediate-risk MDS, low-risk MDS, MDS with single lineage dysplasia refractory anemia, MDS with ring sideroblasts, very low, low or intermediate MDS, anemia associated with MDS, or transfusion dependent anemia associated with MDS, wherein the subject is intolerant, resistant, or refractory to luspatercept.

123. The method of claim 121, wherein the compound of structure (I) is administered orally at a daily dosage of from 10 mg to 350 mg, from 90 mg to 120 mg, 20 mg, 40 mg, 60 mg, 90 mg, 120 mg, 160 mg, 210 mg, or 270 mg; or is administered orally at a weekly dosage of from 10 mg to 350 mg, from 10 mg to 300 mg, from 30 mg to 90 mg, from 30 mg to 300 mg, from 75 mg to 300 mg, from 85 mg to 300 mg, from 95 mg to 300 mg, from 100 mg to 300 mg, 30 mg, 60 mg, 90 mg, or 120 mg; or is administered orally at a maintenance dosage regime.

124. The method of claim 121, wherein the method improves one or more hematologic parameters in the subject, said improvement comprises decreasing myoblasts, increasing hemoglobin, increasing platelets, increasing neutrophils, decreasing hepcidin, reducing units of red blood cell transfused, reducing frequency of transfusion, and/or reducing transfusion dependence.

125. The method of claim 121, further comprising administering an effective amount of one or more therapeutically active agents.

126. The method of claim 121, wherein the compound of structure (I) is formulated in a gelatin capsule comprising about 5 mg, 25 mg, or 125 mg, which is based on free base weight, of the compound of structure (I) and pharmaceutically acceptable carriers comprising microcrystalline cellulose, lactose monohydrate, croscarmellose sodium, magnesium stearate, and a combination thereof.

127. A method for reducing transfusion dependency or frequency in a subject in need thereof, comprising administering to the subject an effective amount of a compound of structure (I): 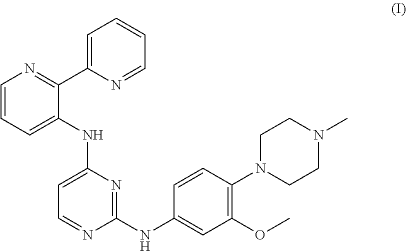 [SMILES: COc1cc(Nc2nccc(Nc3cccnc3-c3ccccn3)n2)ccc1N1CCN(C)CC1]
or a pharmaceutically acceptable salt, or prodrug thereof, wherein the subject is suffering from anemia and has very low, low or intermediate myelodysplastic syndrome (MDS).

128. The method of claim 127, wherein units of red blood cells transfused is reduced by 4 or more units compared to the units of red blood cells transfused for the same period of time prior to administration of the compound of structure (I); wherein the period of time is 8 weeks or 12 weeks.

129. The method of claim 127, wherein the compound of structure (I) is administered orally at a daily dosage of from 10 mg to 350 mg, from 90 mg to 120 mg, 20 mg, 40 mg, 60 mg, 90 mg, 120 mg, 160 mg, 210 mg, or 270 mg; or is administered orally at a weekly dosage of from 10 mg to 350 mg, from 10 mg to 300 mg, from 30 mg to 90 mg, from 30 mg to 300 mg, from 75 mg to 300 mg, from 85 mg to 300 mg, from 95 mg to 300 mg, from 100 mg to 300 mg, 30 mg, 60 mg, 90 mg, or 120 mg; or is administered orally at a maintenance dosage regime.

130. The method of claim 127, wherein the subject has transfusion dependent anemia associated with MDS, MDS with single lineage dysplasia refractory anemia, or MDS with ring sideroblasts and is intolerant, resistant or refractory to luspatercept.

131. The method of claim 127, wherein the method improves one or more hematologic parameters in the subject, said improvement comprises decreasing myoblasts, increasing hemoglobin, increasing platelets, increasing neutrophils, and/or decreasing hepcidin.

132. The method of claim 127, further comprising administering an effective amount of one or more therapeutically active agents.

133. The method of claim 127, wherein the compound of structure (I) is formulated in a gelatin capsule comprising about 5 mg, 25 mg, or 125 mg, which is based on free base weight, of the compound of structure (I) and pharmaceutically acceptable carriers comprising microcrystalline cellulose, lactose monohydrate, croscarmellose sodium, magnesium stearate, and a combination thereof.

134. A method for increasing hemoglobin, increasing platelets, or increasing neutrophils in a subject in need thereof comprising administering to the subject an effective amount of a compound of structure (I): 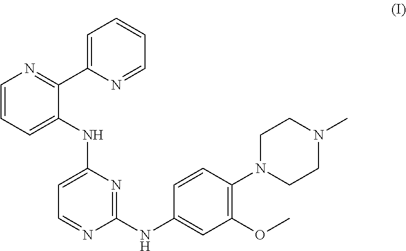 [SMILES: COc1cc(Nc2nccc(Nc3cccnc3-c3ccccn3)n2)ccc1N1CCN(C)CC1]
or a pharmaceutically acceptable salt, or prodrug thereof, wherein the subject is suffering from anemia and has very low, low or intermediate myelodysplastic syndrome (MDS).

135. The method of claim 134, wherein increasing hemoglobin is defined as increasing hemoglobin i) to 10 g/dL or more; or ii) by 1.5 g/dL or more compared to an amount measured prior to administration of the compound of structure (I); wherein the increase in hemoglobin is maintained for 8 weeks or 12 weeks in the absence of red blood cell transfusions.

136. The method of claim 134, wherein increasing platelets is defined as increasing the platelet count i) by 30×10⁹/L or more; or ii) to 75×10⁹/L or more; wherein the increase in platelets is maintained for 8 weeks or 12 weeks in the absence of red blood cell transfusions.

137. The method of claim 134, wherein increasing neutrophils is defined as increasing the neutrophil count i) by 0.5×10⁹/L or more or ii) to 1.0×10⁹/L or more; wherein the increase in neutrophil count is maintained for 8 weeks or 12 weeks in the absence of red blood cell transfusions.

138. The method of claim 134, wherein the compound of structure (I) is administered orally at a daily dosage of from 10 mg to 350 mg, from 90 mg to 120 mg, 20 mg, 40 mg, 60 mg, 90 mg, 120 mg, 160 mg, 210 mg, or 270 mg; or is administered orally at a weekly dosage of from 10 mg to 350 mg, from 10 mg to 300 mg, from 30 mg to 90 mg, from 30 mg to 300 mg, from 75 mg to 300 mg, from 85 mg to 300 mg, from 95 mg to 300 mg, from 100 mg to 300 mg, 30 mg, 60 mg, 90 mg, or 120 mg; or is administered orally at a maintenance dosage regime.

139. The method of claim 134, wherein the subject has transfusion dependent anemia associated with MDS, MDS with single lineage dysplasia refractory anemia, or MDS with ring sideroblasts and is intolerant, resistant or refractory to luspatercept.

140. The method of claim 134, wherein the method improves one or more hematologic parameters in the subject, said improvement comprises decreasing myoblasts, decreasing hepcidin, reducing units of red blood cell transfused, reducing frequency of transfusion, and/or reducing transfusion dependence.

141. The method of claim 134, further comprising administering an effective amount of one or more therapeutically active agents.

142. The method of claim 134, wherein the compound of structure (I) is formulated in a gelatin capsule comprising about 5 mg, 25 mg, or 125 mg, which is based on free base weight, of the compound of structure (I) and pharmaceutically acceptable carriers comprising microcrystalline cellulose, lactose monohydrate, croscarmellose sodium, magnesium stearate, and a combination thereof.

143. A method for decreasing hepcidin or myoblasts in a subject in need thereof comprising administering to the subject an effective amount of a compound of structure (I): 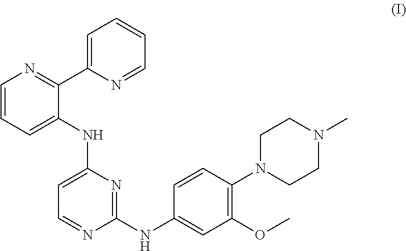 [SMILES: COc1cc(Nc2nccc(Nc3cccnc3-c3ccccn3)n2)ccc1N1CCN(C)CC1]
or a pharmaceutically acceptable salt, or prodrug thereof, wherein the subject is suffering from anemia and has very low, low or intermediate myelodysplastic syndrome (MDS).

144. The method of claim 143, wherein decreasing hepcidin is defined as decreasing hepcidin by 25% or more compared to a baseline amount measured prior to administration of the compound of structure (I).

145. The method of claim 143, wherein decreasing myoblasts is defined as decreasing myoblasts i) to be 5% or fewer of bone marrow cells; or ii) by 50% or more compared to a baseline amount measured prior to administration of the compound of structure (I).

146. The method of claim 143, wherein the compound of structure (I) is administered orally at a daily dosage of from 10 mg to 350 mg, from 90 mg to 120 mg, 20 mg, 40 mg, 60 mg, 90 mg, 120 mg, 160 mg, 210 mg, or 270 mg; or is administered orally at a weekly dosage of from 10 mg to 350 mg, from 10 mg to 300 mg, from 30 mg to 90 mg, from 30 mg to 300 mg, from 75 mg to 300 mg, from 85 mg to 300 mg, from 95 mg to 300 mg, from 100 mg to 300 mg, 30 mg, 60 mg, 90 mg, or 120 mg; or is administered orally at a maintenance dosage regime.

147. The method of claim 143, wherein the method improves one or more hematologic parameters in the subject, said improvement comprises increasing hemoglobin, increasing platelets, increasing neutrophils, reducing units of red blood cell transfused, reducing frequency of transfusion, and/or reducing transfusion dependence.

148. The method of claim 143, wherein the subject has transfusion dependent anemia associated with MDS, MDS with single lineage dysplasia refractory anemia, or MDS with ring sideroblasts and is intolerant, resistant or refractory to luspatercept.

149. The method of claim 143, further comprising administering an effective amount of one or more therapeutically active agents.

150. The method of claim 143, wherein the compound of structure (I) is formulated in a gelatin capsule comprising about 5 mg, 25 mg, or 125 mg, which is based on free base weight, of the compound of structure (I) and pharmaceutically acceptable carriers comprising microcrystalline cellulose, lactose monohydrate, croscarmellose sodium, magnesium stearate, and a combination thereof.

151. A method for treating anemia in a subject in need thereof, wherein the subject has very low, low or intermediate myelodysplastic syndrome (MDS), the method comprising the steps of:
a) determining a baseline amount of hemoglobin or a baseline amount of a biomarker in said subject;
b) administering an effective amount of a compound of structure (I): 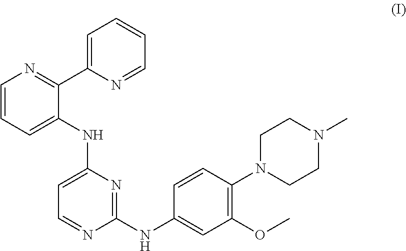 [SMILES: COc1cc(Nc2nccc(Nc3cccnc3-c3ccccn3)n2)ccc1N1CCN(C)CC1]
or a pharmaceutically acceptable salt, or prodrug thereof, to the subject;
c) determining a change in hemoglobin from baseline, or a subsequent level of hemoglobin, or a change in a biomarker level from baseline after said administration step,
wherein if the hemoglobin has increased from baseline by 1.5 g/dL, or if the hemoglobin level is 10 g/dL or more, or if the biomarker has decreased or increased from baseline by a predetermined amount, the method of administering the compound of structure (I) for treatment is determined to be efficacious.

152. The method of claim 151, wherein the biomarker is selected from hepcidin in serum and bone marrow aspirate; iron metabolism markers in serum selected from iron, ferritin, transferrin, soluble transferrin receptor [STR], and total iron binding capacity [TIBC]; cytokines in serum or plasma selected from CRP, EPO, IL-6, and TGF-beta 1; and indicators of inhibition of signal transduction pathways in bone marrow aspirates selected from phosphorylation of SMAD-1, 2, 3, 5 and 8 in PBMCs.

153. The method of claim 151, wherein the compound of structure (I) is administered orally at a daily dosage of from 10 mg to 350 mg, from 90 mg to 120 mg, 20 mg, 40 mg, 60 mg, 90 mg, 120 mg, 160 mg, 210 mg, or 270 mg; or is administered orally at a weekly dosage of from 10 mg to 350 mg, from 10 mg to 300 mg, from 30 mg to 90 mg, from 30 mg to 300 mg, from 75 mg to 300 mg, from 85 mg to 300 mg, from 95 mg to 300 mg, from 100 mg to 300 mg, 30 mg, 60 mg, 90 mg, or 120 mg; or is administered orally at a maintenance dosage regime.

154. The method of claim 151, wherein the subject has transfusion dependent anemia associated with MDS, MDS with single lineage dysplasia refractory anemia, or MDS with ring sideroblasts and is intolerant, resistant or refractory to luspatercept.

155. The method of claim 151, wherein the method improves one or more hematologic parameters in the subject, said improvement comprises decreasing myoblasts, increasing hemoglobin, increasing platelets, increasing neutrophils, decreasing hepcidin, reducing units of red blood cell transfused, reducing frequency of transfusion, and/or reducing transfusion dependence.

156. The method of claim 151, further comprising administering an effective amount of one or more therapeutically active agents.

157. The method of claim 151, wherein the compound of structure (I) is formulated in a gelatin capsule comprising about 5 mg, 25 mg, or 125 mg, which is based on free base weight, of the compound of structure (I) and pharmaceutically acceptable carriers comprising microcrystalline cellulose, lactose monohydrate, croscarmellose sodium, magnesium stearate, and a combination thereof.

158. The method of claim 151, wherein the compound of structure (I) is administered as a loading dosage regime, wherein a predetermined loading dose threshold of hemoglobin is 0.5 g/dL or more.

159. The method of claim 151, wherein the compound of structure (I) is administered as a maintenance dosage regime, wherein the maintenance dose is the dose at which the subject achieves and maintains for a period of time a predetermined threshold level of a hemoblobin or a biomarker.
